(19) United States
(12) Patent Application Publication
(10) Pub. No.: US 2024/0101715 A1
(43) Pub. Date: Mar. 28, 2024

(54) TNF ALPHA AND NGF ANTIBODIES FOR VETERINARY USE

(71) Applicant: Kindred Biosciences, Inc., Burlingame, CA (US)

(72) Inventors: Stephanie A. PIERCE, Burlingame, CA (US); Leonard PRESTA, Burlingame, CA (US); Shyr Jiann LI, Burlingame, CA (US); Lam NGUYEN, Burlingame, CA (US); Richard CHIN, Burlingame, CA (US); Hangjun ZHAN, Burlingame, CA (US)

(21) Appl. No.: 18/258,071

(22) PCT Filed: Dec. 17, 2021
(86) PCT No.: PCT/US2021/064223

§ 371 Date: Jun. 16, 2023

Related U.S. Application Data
(60) Provisional application No. 63/127,994, filed on Dec. 18, 2020.
(60) Provisional application No. 63/128,804, filed on Dec. 21, 2020.

(51) Int. Cl. C07K 16/46; A61P 37/02; G01N 33/68; G01N 33/74

(52) U.S. Cl. CPC C07K 16/468; A61P 37/02; G01N 33/6863; G01N 33/74; C07K 2317/31; C07K 2317/565; C07K 2317/567

(57) ABSTRACT

Provided are various embodiments relating to caninized TNFα antibodies and caninized NGF antibodies. Such antibodies can be used in methods to treat canines with inflammatory conditions, such as inflammatory bowel disease and/or in methods to treat canines with pain, such as osteoarthrititic pain, back pain, cancer pain, and/or a neuropathic pain.

### CROSS REFERENCE TO RELATED APPLICATIONS

This application claims the benefit of priority to U.S. provisional application No. 63/127,994, filed Dec. 18, 2020, and to U.S. provisional application No. 63/128,804, filed Dec. 21, 2020, the contents of each of which are incorporated herein by reference in their entireties.

### FIELD

This present disclosure relates to caninized TNF alpha antibodies and/or caninized NGF antibodies, for example, for treating inflammatory conditions in canines, such as inflammatory bowel disease, and/or for treating pain in canines.

### BACKGROUND

TNF is a cytokine involved in systemic inflammation and stimulates the acute phase reaction. TNF promotes the inflammatory response, which, in turn, causes or contributes to many of the clinical problems associated with autoimmune disorders such ankylosing spondylitis, asthma, cancer, Crohn's disease, inflammatory bowel disease (IBD), juvenile idiopathic arthritis, psoriasis, including plaque psoriasis, psoriatic arthritis, rheumatoid arthritis, ulcerative colitis, and other chronic inflammatory disorders.

The present disclosure provides methods and compositions for treating diseases such as IBD in companion animals with TNFα antibodies, and so relates to the fields of biology, molecular biology, and veterinary medicine.

Companion animals such as cats, dogs, and horses, suffer from many diseases similar to human diseases, including autoimmune diseases, cancer and chronic inflammatory disorders. However, there has been no demonstration to date that adalimumab or infliximab could be used to treat such diseases in companion animals. Moreover, proteins with significant human-derived amino acid sequence content, especially if those sequences are derived from antibodies, can be immunogenic in non-human animals. And human drugs that bind human TNF might not bind companion animal TNF in a manner that provides an equally beneficial therapeutic effect in the companion animal as it does in the human, especially if the companion animal TNF differs in sequence from human TNF. Similarly, if the human drug elicits an immune response in the companion animal, it may not be effective. See Mauldin et al., Aug. 2010, 21(4):373-382.

IBD is a collective term used to describe a disorder of the gastrointestinal tract of dogs with associated histologic evidence of inflammation. See Ettinger S J and Feldman E C. Diseases of the Small Intestine. Textbook of Veterinary Internal Medicine: Diseases of the Dog and the Cat, Elsevier Saunders, 2010. While the exact etiology of canine IBD remains unknown, alterations in immune system tolerance to dietary antigens and intestinal bacteria are thought to play a major role, as is genetic susceptibility. Canine IBD may share a similar etiology with the disorder that occurs in humans (also known as Crohn's disease and ulcerative colitis), but the clinical syndrome and histologic changes associated with dogs and humans are different. See Ettinger and Feldman; Suchodolski J S et al. “The fecal microbiome in dogs with acute diarrhea and idiopathic inflammatory bowel disease.” PLoS ONE. 2012; 7(12): e51907. doi: 10.1371/journal.pone.0051907; Suchodolski J S. “Companion-animals symposium: Microbes and gastrointestinal health of dogs and cats.” J Anim Sci. 2010; 89(5):1520-1530. Clinically, canine IBD is characterized by persistent or recurrent signs such as vomiting, diarrhea, abdominal pain, weight loss, or alterations in appetite.

Accurate diagnosis of canine IBD can be challenging and the term “idiopathic” is often used when an exact causative agent cannot be identified. A complete history and physical exam followed by laboratory investigation, diagnostic imaging, and intestinal biopsy (to demonstrate the presence of inflammation) are typically recommended. Further, dogs with lymphocytic-plasmacytic colitis (LPC), a common form of IBD, failed to express a predominant cytokine profile (including TNFα) in inflamed colonic mucosa as opposed to human IBD. See Tamura Y et al. “Evaluation of Selected Cytokine Gene Expression in Colonic Mucosa from Dogs with Idiopathic Lymphocytic-plasmacytic Colitis.” J Vet Med Sci. 2014; 76(10):1407-10. Therapeutic strategies to manage these patients include anti-parasiticides (dewormer), antibiotics, dietary modification, and administration of immuno-suppressive medications with remission of clinical signs being the goal. See Ettinger and Feldman. In humans, anti-inflammatory monoclonal antibodies which inhibit TNF are widely used to treat this disease. In our canine patients, the prognosis and response to traditional therapy varies and can range from excellent to poor. Therefore, treatment outcomes in dog are not predictable based on human treatment.

There remains a need, therefore, for methods and compounds that can be used to bind companion animal TNF in companion animals for treating TNF associated conditions in companion animals. Ideally, the compounds would bind specifically to companion animal TNF and have a half-life in plasma sufficiently long to be practicable for therapy, but would not be highly immunogenic in companion animals. The present invention meets this need.

The present disclosure also provides methods and compositions for pain such as chronic pain or inflammatory pain in companion animals with NGF antibodies, and so relates to the fields of biology, molecular biology, and veterinary medicine.

Nerve growth factor (NGF) is a neurotrophic factor with broad effect on regulation of growth, maintenance, proliferation, and survival of certain neurons. NGF has also been linked to chronic and inflammatory pain. NGF binds to two classes of receptors: the tropomyosine receptor kinase A (TrkA) and low affinity NGF receptor. When NGF, a dimer, binds to TrkA extracellular domains, it causes the dimerization of the receptor, activating the downstream kinase activity. NGF antibodies may be useful to antagonize NGF activity, reduce free NGF, and/or diminishing clinical signs and symptoms associated with NGF-related pain.

Companion species animals, such as cats, dogs, and horses, may suffer from chronic and inflammatory pain. There remains a need for methods and species-specific compounds that can be used specifically to bind companion animal NGF for treating NGF-induced conditions and for reducing NGF signaling activity.

The present disclosure also provides for bi-specific antibodies capable of binding both TNF and NGF, as well as methods for using such molecules for treatment. For example, the bi-specific molecules of the present disclosure may be used for treating both inflammation and the pain associated with it, such as for treating osteoarthritis, chronic pain, low back pain, cancer pain, and neuropathic pain.

### SUMMARY

Embodiment 1. An isolated antibody that binds to canine TNFα, wherein the antibody is a caninized antibody comprising a variable light chain comprising (i) a CDR-L1 comprising the amino acid sequence of SEQ ID NO: 1; (ii) a CDR-L2 comprising the amino acid sequence of SEQ ID NO: 2; (iii) a CDR L3 comprising the amino acid sequence of SEQ ID NO: 3; and (iv) a LC-FR2 comprising a glutamine at position 3 and a lysine at position 8.

Embodiment 2. The isolated antibody of embodiment 1, wherein the antibody comprises a variable heavy chain comprising (i) a CDR-H1 comprising the amino acid sequence of SEQ ID NO: 4; (ii) a CDR-H2 comprising the amino acid sequence of SEQ ID NO: 5; and (iii) a CDR H3 comprising the amino acid sequence of SEQ ID NO: 6.

Embodiment 3. The isolated antibody of any one of the preceding embodiments, wherein the LC-FR2 comprises the amino acid sequence of SEQ ID NO: 45.

Embodiment 4. The isolated antibody of any one of the preceding embodiments, wherein the antibody comprises (i) a variable light chain comprising the amino acid sequence of SEQ ID NO: 43 or SEQ ID NO: 46 or a variant thereof wherein 1, 2, 3, 4, 5, or 6 amino acids of the variable light chain is substituted by a different amino acid, (ii) a variable heavy chain comprising the amino acid sequence of SEQ ID NO: 22 or a variant thereof wherein 1, 2, 3, 4, 5, or 6 amino acids of the variable heavy chain is substituted by a different amino acid, or (iii) a variable light chain as in (i) and a variable heavy chain as in (ii).

Embodiment 5. The isolated antibody of any one of the preceding embodiments, wherein the antibody comprises (i) a variable light chain comprising the amino acid sequence of SEQ ID NO: 43 or SEQ ID NO: 46 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 43 or SEQ ID NO: 46, (ii) a variable heavy chain comprising the amino acid sequence of SEQ ID NO: 22 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 22, or (iii) a variable light chain as in (i) and a variable heavy chain as in (ii).

Embodiment 6. An isolated antibody that binds to canine TNFα, wherein the antibody comprises: (i) a variable light chain comprising the amino acid sequence of SEQ ID NO: 14, SEQ ID NO: 18, SEQ ID NO: 25, SEQ ID NO: 29, SEQ ID NO: 33, SEQ ID NO: 37, SEQ ID NO: 43, or SEQ ID NO: 46; (ii) a variable heavy chain comprising the amino acid sequence of SEQ ID NO: 22; or (iii) a variable light chain as in (i) and a variable heavy chain as in (ii).

Embodiment 7. An isolated antibody that binds to canine TNFα, wherein the antibody comprises a variable light chain comprising the amino acid sequence of SEQ ID NO: 14, SEQ ID NO: 18, SEQ ID NO: 25, SEQ ID NO: 29, SEQ ID NO: 33, SEQ ID NO: 37, SEQ ID NO: 43, or SEQ ID NO: 46 and a variable heavy chain comprising the amino acid sequence of SEQ ID NO: 22.

Embodiment 8. An isolated antibody that binds to canine TNFα, wherein the antibody comprises a variable light chain comprising the amino acid sequence of SEQ ID NO: 43 and a variable heavy chain comprising the amino acid sequence of SEQ ID NO: 22.

Embodiment 9. An isolated antibody that binds to canine TNFα, wherein the antibody comprises a variable light chain comprising the amino acid sequence of SEQ ID NO: 46 and a variable heavy chain comprising the amino acid sequence of SEQ ID NO: 22.

Embodiment 10. An isolated antibody that binds to canine TNFα, wherein the antibody is a caninized antibody comprising:
(a) a light chain comprising (i) a CDR-L1 comprising the amino acid sequence of SEQ ID NO: 49; (ii) a CDR-L2 comprising the amino acid sequence of SEQ ID NO: 50; (iii) a CDR L3 comprising the amino acid sequence of SEQ ID NO: 51; and (iv) a variable light chain comprising the amino acid sequence of SEQ ID NO: 55 or SEQ ID NO: 56 or a variant thereof wherein 1, 2, 3, 4, 5, or 6 amino acids of the variable light chain is substituted by a different amino acid;
(b) a heavy chain comprising (i) a CDR-H1 comprising the amino acid sequence of SEQ ID NO: 52; (ii) a CDR-H2 comprising the amino acid sequence of SEQ ID NO: 53; (iii) a CDR H3 comprising the amino acid sequence of SEQ ID NO: 54; and (iv) a variable heavy chain comprising the amino acid sequence of SEQ ID NO: 57 or SEQ ID NO: 58 or a variant thereof wherein 1, 2, 3, 4, 5, or 6 amino acids of the variable heavy chain is substituted by a different amino acid; or
(c) a light chain as in (a) and a heavy chain as in (b).

Embodiment 11. An isolated antibody that binds to canine TNFα, wherein the antibody is a caninized antibody comprising:
(a) a light chain comprising (i) a CDR-L1 comprising the amino acid sequence of SEQ ID NO: 49; (ii) a CDR-L2 comprising the amino acid sequence of SEQ ID NO: 50; (iii) a CDR L3 comprising the amino acid sequence of SEQ ID NO: 51; and (iv) a variable light chain comprising the amino acid sequence of SEQ ID NO: 55 or SEQ ID NO: 56 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 55 or SEQ ID NO: 56;
(b) a heavy chain comprising (i) a CDR-H1 comprising the amino acid sequence of SEQ ID NO: 52; (ii) a CDR-H2 comprising the amino acid sequence of SEQ ID NO: 53; (iii) a CDR H3 comprising the amino acid sequence of SEQ ID NO: 54; and (iv) a variable heavy chain comprising the amino acid sequence of SEQ ID NO: 57 or SEQ ID NO: 58 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 57 or SEQ ID NO: 58; or
(c) a light chain as in (a) and a heavy chain as in (b).

Embodiment 12. An isolated antibody that binds to canine TNFα, wherein the antibody comprises: (i) a variable light chain comprising the amino acid sequence of SEQ ID NO: 55 or SEQ ID NO: 56; (ii) a variable heavy chain comprising the amino acid sequence of SEQ ID NO: 57 or SEQ ID NO: 58; or (iii) a variable light chain as in (i) and a variable heavy chain as in (ii).

Embodiment 13. The antibody of any one of the preceding embodiments, wherein the antibody comprises a canine constant heavy chain region and/or a canine constant light chain region.

Embodiment 14. The antibody of any one of the preceding embodiments, wherein the antibody comprises a canine heavy chain constant region selected from an IgG-A, IgG-B, IgG-C, and IgG-D constant region.

Embodiment 15. The isolated antibody of any one of embodiments 1 to 9, 13, or 14, wherein the antibody comprises: (i) a light chain comprising the amino acid sequence of SEQ ID NO: 15, SEQ ID NO: 19, SEQ ID NO: 26, SEQ ID NO: 30, SEQ ID NO: 34, SEQ ID NO: 38, or SEQ ID NO: 44 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 15, SEQ ID NO: 19, SEQ ID NO: 26, SEQ ID NO: 30, SEQ ID NO: 34, SEQ ID NO: 38, or SEQ ID NO: 44, (ii) a heavy chain comprising the amino acid sequence of SEQ ID NO: 23 or SEQ ID NO: 64 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 23 or SEQ ID NO: 64, or (iii) a light chain as in (i) and a heavy chain as in (ii).

Embodiment 16. The isolated antibody of any one of embodiments 1 to 9 or embodiments 13 to 15, wherein the antibody comprises: (i) a light chain comprising the amino acid sequence of SEQ ID NO: 44, (ii) a heavy chain comprising the amino acid sequence of SEQ ID NO: 23 or SEQ ID NO: 64, or (iii) a light chain as in (i) and a heavy chain as in (ii).

Embodiment 17. An isolated antibody that binds to canine TNFα, wherein the antibody comprises a light chain comprising the amino acid sequence of SEQ ID NO: 44 and a heavy chain comprising the amino acid sequence of SEQ ID NO: 23 or SEQ ID NO: 64.

Embodiment 18. An isolated antibody that binds to canine TNFα, wherein the antibody comprises a light chain comprising the amino acid sequence of SEQ ID NO: 15 and a heavy chain comprising the amino acid sequence of SEQ ID NO: 23 or SEQ ID NO: 64.

Embodiment 19. An isolated antibody that binds to canine TNFα, wherein the antibody is a caninized antibody comprising:

(a) a light chain comprising (i) a CDR-L1 comprising the amino acid sequence of SEQ ID NO: 49; (ii) a CDR-L2 comprising the amino acid sequence of SEQ ID NO: 50; (iii) a CDR L3 comprising the amino acid sequence of SEQ ID NO: 51; and (iv) a light chain comprising the amino acid sequence of SEQ ID NO: 59 or SEQ ID NO: 60 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 59 or SEQ ID NO: 60;
(b) a heavy chain comprising (i) a CDR-H1 comprising the amino acid sequence of SEQ ID NO: 52; (ii) a CDR-H2 comprising the amino acid sequence of SEQ ID NO: 53; (iii) a CDR H3 comprising the amino acid sequence of SEQ ID NO: 54; and (iv) a heavy chain comprising the amino acid sequence of SEQ ID NO: 61 or SEQ ID NO: 62 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 61 or SEQ ID NO: 62; or
(c) a light chain as in (a) and a heavy chain as in (b).

Embodiment 20. An isolated antibody that binds to canine TNFα, wherein the antibody comprises a light chain comprising the amino acid sequence of SEQ ID NO: 59 or SEQ ID NO: 60 and a heavy chain comprising the amino acid sequence of SEQ ID NO: 61 or SEQ ID NO:

62.

Embodiment 21. An isolated antibody that binds to canine NGF, wherein the antibody is a caninized antibody comprising (i) a CDR-H1 comprising the amino acid sequence of SEQ ID NO: 75; (ii) a CDR-H2 comprising the amino acid sequence of SEQ ID NO: 69, and (iii) a CDR-H3 comprising the amino acid sequence of SEQ ID NO: 70.

Embodiment 22. The isolated antibody of embodiment 21, wherein the antibody further comprises (iv) a CDR-L1 comprising the amino acid sequence of SEQ ID NO: 65; (v) a CDR-L2 comprising the amino acid sequence of SEQ ID NO: 66; and (vi) a CDR L3 comprising the amino acid sequence of SEQ ID NO: 67.

Embodiment 23. The isolated antibody of embodiment 21 or embodiment 22, wherein the antibody comprises (i) a variable light chain comprising the amino acid sequence of SEQ ID NO: 73, a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 73; (ii) a variable heavy chain comprising the amino acid sequence of SEQ ID NO: 74 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 74; or (iii) a variable heavy chain as in (i) and a variable heavy chain as in (ii).

Embodiment 24. An isolated antibody that binds to canine NGF, wherein the antibody is a caninized antibody comprising (i) a variable light chain comprising the amino acid sequence of SEQ ID NO: 73, a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 73; (ii) a variable heavy chain comprising the amino acid sequence of SEQ ID NO: 74 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 74; or (iii) a variable heavy chain as in (i) and a variable heavy chain as in (ii).

Embodiment 25. The isolated antibody of any one of embodiments 21 to 24, wherein the antibody comprises (i) a variable light chain sequence of 73 or a variant thereof wherein 1, 2, 3, 4, 5, or 6 amino acids of the variable light chain is substituted by a different amino acid, (ii) a variable heavy chain sequence of SEQ ID NO: 74 or a variant thereof wherein 1, 2, 3, 4, 5, or 6 amino acids of the variable heavy chain is substituted by a different amino acid, or (iii) a variable light chain sequence as in (i) and a variable heavy chain sequence as in (ii).

Embodiment 26. The antibody of any one of embodiments 21 to 25, wherein the antibody comprises a canine constant heavy chain region and/or a canine constant light chain region.

Embodiment 27. The antibody of any one of embodiments 21 to 26, wherein the antibody comprises a canine heavy chain constant region selected from an IgG-A, IgG-B, IgG-C, and IgG-D constant region.

Embodiment 28. The isolated antibody of any one of embodiments 21 to 27, wherein the antibody comprises: (i) a light chain sequence of SEQ ID NO: 77 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 77, (ii) a heavy chain sequence of SEQ ID NO: 79 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 79, or (iii) a light chain sequence as in (i) and a heavy chain sequence as in (ii).

Embodiment 29. The isolated antibody of any one of embodiments 21 to 28, wherein the antibody comprises a light chain comprising the amino acid sequence of SEQ ID NO: 77 and a heavy chain comprising the amino acid sequence of SEQ ID NO: 79.

Embodiment 30. An isolated antibody that binds to canine NGF, wherein the antibody comprises a light chain comprising the amino acid sequence of SEQ ID NO: 77 and a heavy chain comprising the amino acid sequence of SEQ ID NO: 79.

Embodiment 31. The antibody of any one of the preceding embodiments, wherein the antibody is an antibody fragment selected from Fv, scFv, Fab, Fab′, F(ab′)2, and Fab′-SH.

Embodiment 32. An isolated antibody that binds to canine TNFα and canine NGF, wherein the antibody comprises:

(i) a variable light chain comprising the amino acid of SEQ ID NO: 14, SEQ ID NO: 18, SEQ ID NO: 25, SEQ ID NO: 29, SEQ ID NO: 33, SEQ ID NO: 37, SEQ ID NO: 43, SEQ ID NO: 46, SEQ ID NO: 55, or SEQ ID NO: 56 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: SEQ ID NO: 14, SEQ ID NO: 18, SEQ ID NO: 25, SEQ ID NO: 29, SEQ ID NO: 33, SEQ ID NO: 37, SEQ ID NO: 43, SEQ ID NO: 46, SEQ ID NO: 55, or SEQ ID NO: 56;
(ii) a variable heavy chain comprising the amino acid sequence of SEQ ID NO: 22, SEQ ID NO: 57, or SED NO: 58 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 22, SEQ ID NO: 57, or SEQ ID NO: 58;
(iii) a variable light chain comprising the amino acid sequence of SEQ ID NO: 73 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 73;
(iv) a variable heavy chain comprising the amino acid sequence of SEQ ID NO: 74 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 74; or
(v) a variable light chain sequence as in (i), a variable heavy chain sequence as in (ii), a variable light chain sequence as in (iii), and a variable heavy chain sequence as in (iv).

Embodiment 33. The isolated antibody of embodiment 32, wherein the antibody comprises:
(i) a variable light chain comprising the amino acid of SEQ ID NO: 43 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 43;
(ii) a variable heavy chain comprising the amino acid sequence of SEQ ID NO: 22 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 22;
(iii) a variable light chain comprising the amino acid sequence of SEQ ID NO: 73 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 73;
(iv) a variable heavy chain comprising the amino acid sequence of SEQ ID NO: 74 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 74; or
(v) a variable light chain sequence as in (i), a variable heavy chain sequence as in (ii), a variable light chain sequence as in (iii), and a variable heavy chain sequence as in (iv).

Embodiment 34. An isolated antibody that binds to canine TNFα and canine NGF, wherein the antibody comprises: (i) a variable light chain comprising the amino acid of SEQ ID NO: 43; (ii) a variable heavy chain comprising the amino acid sequence of SEQ ID NO: 22; (iii) a variable light chain comprising the amino acid sequence of SEQ ID NO: 73; and (iv) a variable heavy chain comprising the amino acid sequence of SEQ ID NO: 74.

Embodiment 35. The isolated antibody of embodiment 33, wherein the antibody comprises the amino acid sequence of SEQ ID NO: 77 and the amino acid sequence of SEQ ID NO: 81.

Embodiment 36. An isolated nucleic acid or nucleic acids encoding the antibody of any one of embodiments 1 to 35.

Embodiment 37. A host cell comprising the nucleic acid or nucleic acids of embodiment 36.

Embodiment 38. A method of producing an antibody comprising culturing the host cell of embodiment 37 and isolating the antibody.

Embodiment 39. A pharmaceutical composition comprising the antibody of any one of embodiments 1 to 35 and a pharmaceutically acceptable carrier.

Embodiment 40. A method of treating a canine having a condition associated with TNFα, the method comprising administering to the canine a therapeutically effective amount of the antibody of any one of embodiments 1 to 20 and 31 to 35 or the pharmaceutical composition of embodiment 39.

Embodiment 41. A method of maintaining remission of a condition associated with TNFα in a canine, the method comprising administering to the canine a therapeutically effective amount of the antibody of any one of embodiments 1 to 20 and 31 to 35 or the pharmaceutical composition of embodiment 37.

Embodiment 42. The method of embodiment 40 or embodiment 41, wherein the condition associated with TNFα is an inflammatory disease.

Embodiment 43. The method of any one of embodiments 40 to 42, wherein the condition associated with TNFα is a gastrointestinal inflammatory disease.

Embodiment 44. The method of any one of embodiments 40 to 43, wherein the condition associated with TNFα is inflammatory bowel disease.

Embodiment 45. The method of any one of embodiments 40 to 44, wherein the condition associated with TNFα is ankylosing spondylitis, asthma, cancer, Crohn's disease, idiopathic arthritis, psoriasis, plaque psoriasis, psoriatic arthritis, rheumatoid arthritis, or ulcerative colitis.

Embodiment 46. A method of treating a canine having a condition associated with NGF, the method comprising administering to the canine a therapeutically effective amount of the antibody of any one of embodiments 21 to 35 or the pharmaceutical composition of embodiment 39.

Embodiment 47. A method of maintaining remission of a condition associated with NGF in a canine, the method comprising administering to the canine a therapeutically effective amount of the antibody of any one of embodiments 21 to 35 or the pharmaceutical composition of embodiment 39.

Embodiment 48. The method of treating pain in a canine, the method comprising administering to the canine a therapeutically effective amount of the antibody of any one of embodiments 21 to 35 or the pharmaceutical composition of embodiment 39.

Embodiment 49. The method of any one of embodiments 46 to 48, wherein the condition associated with NGF or the pain is chronic pain, acute pain, and/or inflammatory pain.

Embodiment 50. The method of any one of embodiments 46 to 49, wherein the condition associated with NGF or the pain is osteoarthrititic pain, back pain, cancer pain, and/or a neuropathic pain.

Embodiment 51. The method of any one of embodiments 46 to 50, wherein the condition associated with NGF or the pain is pain associated with a surgery, a broken or fractured bone, dental work, a burn, a cut, and/or labor.

Embodiment 52. The method of any one of embodiments 40 to 51, wherein the antibody or the pharmaceutical composition is administered parenterally.

Embodiment 53. The method of any one of embodiments 40 to 52, wherein the antibody or the pharmaceutical composition is administered by an intramuscular route, an intraperitoneal route, an intracerebrospinal route, a subcutaneous route, an intra-arterial route, an intrasynovial route, an intrathecal route, or an inhalation route.

Embodiment 54. The method of any one of embodiments 40 to 53, wherein the method further comprises administering an IL17 antibody, an IL-5 antibody, an IL-31 antibody, an IL4 antibody, an IL13 antibody, an IL23 antibody, an IgE antibody, a CD11α antibody, an IL6R antibody, an α4-Intergrin antibody, an IL12 antibody, an IL1β antibody, or an anti-BlyS antibody.

Embodiment 55. The method of any one of embodiments 40 to 54, wherein the method further comprises administering an NGF kinase inhibitor, a PI3K inhibitor, a ras inhibitor, a CGRP inhibitor, a TNF inhibitor, an IL17 inhibitor, an EGFR inhibitor, and/or a Phospholipase C pathway inhibitor.

Embodiment 56. The method of any one of embodiments 40 to 55, wherein the method further comprises administering one or more pain therapy drugs, such as a corticosteroid, a non-steroidal anti-inflammatory drug (NSAID), a cyclooxygenase inhibitor, an opioid, and/or a cannabinoid.

Embodiment 57. The method of any one of embodiments 40 to 56, wherein the antibody is administered at an amount in the range of 0.01 mg/kg body weight to 100 mg/kg body weight per dose.

Embodiment 58. The method of any one of embodiments 40 to 57, wherein the antibody is administered at a dose of 2 mg/kg body weight.

Embodiment 59. A method of reducing TNFα and/or NGF signaling function in a cell, the method comprising exposing to the cell the antibody of any one of embodiments 1 to 35 or the pharmaceutical composition of embodiment 39 under conditions permissive for binding of the antibody to TNFα and/or NGF, thereby reducing binding to TNFα and/or NGF signaling function by the cell.

Embodiment 60. The method of embodiment 59, wherein the cell is exposed to the antibody or the pharmaceutical composition ex vivo.

Embodiment 61. The method of embodiment 59, wherein the cell is exposed to the antibody or the pharmaceutical composition in vivo.

Embodiment 62. The method of any one of embodiments 59 to 61, wherein the cell is a canine cell, a feline cell, or an equine cell.

Embodiment 63. A method for detecting TNFα and/or NGF in a sample from a companion animal species comprising contacting the sample with the antibody of any one of embodiments 1 to 35 or the pharmaceutical composition of embodiment 39 under conditions permissive for binding of the antibody to TNFα and/or NGF, and detecting whether a complex is formed between the antibody and TNFα and/or NGF in the sample.

Embodiment 64. The method of embodiment 63, wherein the sample is a biological sample obtained from a canine, a feline, or an equine.

### BRIEF DESCRIPTION OF THE DRAWINGS

FIG. 1 is an alignment of variable light chain sequences of D2E7, KBA VL v1, KBA VL v2, KBA VL v3, KBA VL v4, KBA VL v5, KBA VL v6, and KBA VL v7 and an alignment of variable heavy chain sequences of D2E7 and KBA.

DESCRIPTION OF CERTAIN SEQUENCES

Table 1 provides a listing of certain sequences referenced herein.

TABLE 1
Description of the Sequences

| SEQ ID | | |
|---|---|---|
| NO: | SEQUENCE | DESCRIPTION |
| 1 | RASQGIRNYLA | Variable light chain |
| | | CDR-L1 of D2E7 |
| 2 | AASTLQ | Variable light chain |
| | | CDR-L2 of D2E7 |
| 3 | QRYNRAPYT | Variable light chain |
| | | CDR-L3 of D2E7 |
| 4 | FTFDDYAMH | Variable heavy chain |
| | | CDR-H1 of D2E7 |
| 5 | AITWNSGHIDYADSVEGR | Variable heavy chain |
| | | CDR-H2 of D2E7 |
| 6 | AKVSYLSTASSLDY | Variable heavy chain |
| | | CDR-H3 of D2E7 |
| 7 | DIQMTQSPSSLSASVGDRVTITCRASQGIRNY | Variable light chain of |
| | LAWYQQKPGKAPKLLIYAASTLQSGVPSRFSG | D2E7 |
| | SGSGTDFTLTISSLQPEDVATYYCQRYNRAPY | |
| | TFGQGTKVEIK | |
| 8 | EVQLVESGGGLVQPGRSLRLSCAASGFTFDDY | Variable heavy chain of |
| | AMHWVRQAPGKGLEWVSAITWNSGHIDYADSV | D2E7 |
| | EGRFTISRDNAKNSLYLQMNSLRAEDTAVYYC | |
| | AKVSYLSTASSLDYWGQGTLVTVSS | |
| 9 | METDTLLLWVLLLWVPGSTGDIQMTQSPSSLS | Chimeric variable light |
| | ASVGDRVTITCRASQGIRNYLAWYQQKPGKAP | chain of D2E7 and canine |
| | KLLIYAASTLQSGVPSRFSGSGSGTDFTLTIS | kappa light constant region |
| | SLQPEDVATYYCQRYNRAPYTFGQGTKVEIKR | with leader |
| | NDAQPAVYLFQPSPDQLHTGSASVVCLLNSFY | |
| | PKDINVKWKVDGVIQDTGIQESVTEQDKDSTY | |
| | SLSSTLTMSSTEYLSHELYSCEITHKSLPSTL | |
| | IKSFQRSECQRVD | |
| 10 | DIQMTQSPSSLSASVGDRVTITCRASQGIRNY | Chimeric variable light |
| | LAWYQQKPGKAPKLLIYAASTLQSGVPSRFSG | chain of D2E7 and canine |
| | SGSGTDFTLTISSLQPEDVATYYCQRYNRAPY | kappa light constant region |
| | TFGQGTKVEIKRNDAQPAVYLFQPSPDQLHTG | |
| | SASVVCLLNSFYPKDINVKWKVDGVIQDTGIQ | |
| | ESVTEQDKDSTYSLSSTLTMSSTEYLSHELYS | |
| | CEITHKSLPSTLIKSFQRSECQRVD | |
| 11 | MAVLGLLLCLVTFPSCVLSEVQLVESGGGLVQ | Chimeric variable heavy |
| | PGRSLRLSCAASGFTFDDYAMHWVRQAPGKGL | chain of D2E7 and canine |
| | EWVSAITWNSGHIDYADSVEGRFTISRDNAKN | IgG-B constant region with |
| | SLYLQMNSLRAEDTAVYYCAKVSYLSTASSLD | leader |
| | YWGQGTLVTVSSASTTAPSVFPLAPSCGSTSG | |
| | STVALACLVSGYFPEPVTVSWNSGSLTSGVHT | |
| | FPSVLQSSGLYSLSSMVTVPSSRWPSETFTCN | |
| | VAHPASKTKVDKPVPKRENGRVPRPPDCPKCP | |
| | APEMLGGPSVFIFPPKPKDTLLIARTPEVTCV | |
| | VVDLDPEDPEVQISWFVDGKQMQTAKTQPREE | |
| | QFNGTYRVVSVLPIGHQDWLKGKQFTCKVNNK | |
| | ALPSPIERTISKARGQAHQPSVYVLPPSREEL | |
| | SKNTVSLTCLIKDFFPPDIDVEWQSNGQQEPE | |
| | SKYRTTPPQLDEDGSYFLYSKLSVDKSRWQRG | |
| | DTFICAVMHEALHNHYTQESLSHSPGK | |
| 12 | EVQLVESGGGLVQPGRSLRLSCAASGFTEDDY | Chimeric variable heavy |
| | AMHWVRQAPGKGLEWVSAITWNSGHIDYADSV | chain of D2E7 and canine |
| | EGRFTISRDNAKNSLYLQMNSLRAEDTAVYYC | IgG-B constant region |
| | AKVSYLSTASSLDYWGQGTLVTVSSASTTAPS | |
| | VFPLAPSCGSTSGSTVALACLVSGYFPEPVTV | |
| | SWNSGSLTSGVHTFPSVLQSSGLYSLSSMVTV | |
| | PSSRWPSETFTCNVAHPASKTKVDKPVPKREN | |
| | GRVPRPPDCPKCPAPEMLGGPSVFIFPPKPKD | |
| | TLLIARTPEVTCVVVDLDPEDPEVQISWFVDG | |
| | KQMQTAKTQPREEQFNGTYRVVSVLPIGHQDW | |
| | LKGKQFTCKVNNKALPSPIERTISKARGQAHQ | |
| | PSVYVLPPSREELSKNTVSLTCLIKDFFPPDI | |
| | DVEWQSNGQQEPESKYRTTPPQLDEDGSYFLY | |
| | SKLSVDKSRWQRGDTFICAVMHEALHNHYTQE | |
| | SLSHSPGK | |
| 13 | MDMRVPAQLLGLLLLWLRGARCDIVMTQTPLS | Caninized variable light |
| | LSASPGEPVSISCRASQGIRNYLAWYLQKPGQ | chain of D2E7 v1 and |
| | SPRLLIYAASTLQSGVPDRFSGSGSGTDFTLR | canine kappa light constant |
| | IGRLEAEDAAIYYCQRYNRAPYTFGQGTKVEI | region with leader |
| | KRNDAQPAVYLFQPSPDQLHTGSASVVCLLNS | |
| | FYPKDINVKWKVDGVIQDTGIQESVTEQDKDS | |
| | TYSLSSTLTMSSTEYLSHELYSCEITHKSLPS | |
| | TLIKSFQRSECQRVD | |
| 14 | DIVMTQTPLSLSASPGEPVSISCRASQGIRNY | Caninized variable light |
| | LAWYLQKPGQSPRLLIYAASTLQSGVPDRFSG | chain of D2E7 v1 |
| | SGSGTDFTLRIGRLEAEDAAIYYCQRYNRAPY | (KBA VL v1) |
| | TFGQGTKVEIK | |
| 15 | DIVMTQTPLSLSASPGEPVSISCRASQGIRNY | Caninized variable light |
| | LAWYLQKPGQSPRLLIYAASTLQSGVPDRFSG | chain of D2E7 v1 and |
| | SGSGTDFTLRIGRLEAEDAAIYYCQRYNRAPY | canine kappa light constant |
| | TFGQGTKVEIKRNDAQPAVYLFQPSPDQLHTG | region |
| | SASVVCLLNSFYPKDINVKWKVDGVIQDTGIQ | |
| | ESVTEQDKDSTYSLSSTLTMSSTEYLSHELYS | |
| | CEITHKSLPSTLIKSFQRSECQRVD | |
| 16 | WYLQKPGQSPRLLIY | Variable light chain |
| | | framework region LC-FR2 |
| | | of KBA VL v1 |
| 17 | MDMRVPAQLLGLLLLWLRGARCDIVMTQTPPS | Caninized variable light |
| | LSASPGEPVSISCRASQGIRNYLAWFRQKPGQ | chain of D2E7 v2 and |
| | SPQLLIYAASTLQSGVSDRFSGSGSGTDFTLR | canine kappa light constant |
| | ISRLEADDAAVYYCQRYNRAPYTFGQGTKVEI | region with leader |
| | KRNDAQPAVYLFQPSPDQLHTGSASVVCLLNS | |
| | FYPKDINVKWKVDGVIQDTGIQESVTEQDKDS | |
| | TYSLSSTLTMSSTEYLSHELYSCEITHKSLPS | |
| | TLIKSFQRSECQRVD | |
| 18 | DIVMTQTPPSLSASPGEPVSISCRASQGIRNY | Caninized variable light |
| | LAWFRQKPGQSPQLLIYAASTLQSGVSDRFSG | chain of D2E7 v2 |
| | SGSGTDFTLRISRLEADDAAVYYCQRYNRAPY | (KBA VL v2) |
| | TFGQGTKVEIK | |
| 19 | DIVMTQTPPSLSASPGEPVSISCRASQGIRNY | Caninized variable light |
| | LAWFRQKPGQSPQLLIYAASTLQSGVSDRFSG | chain of D2E7 v2 and |
| | SGSGTDFTLRISRLEADDAAVYYCQRYNRAPY | canine kappa light constant |
| | TFGQGTKVEIKRNDAQPAVYLFQPSPDQLHTG | region |
| | SASVVCLLNSFYPKDINVKWKVDGVIQDTGIQ | |
| | ESVTEQDKDSTYSLSSTLTMSSTEYLSHELYS | |
| | CEITHKSLPSTLIKSFQRSECQRVD | |
| 20 | WFRQKPGQSPQLLIY | Variable light chain |
| | | framework region LC-FR2 |
| | | of KBA VL v2 |
| 21 | MGWSWIFLFLLSGTAGVHSEVQLVESGGDLVK | Exemplary caninized |
| | PGGSLRLSCVASGFTFDDYAMHWVRQAPGKGL | variable heavy chain of |
| | QWVSAITWNSGHIDYADAVKGRFTISRDNAKN | D2E7 and canine IgG-B |
| | TLYLQMNSLRAEDTAVYYCAKVSYLSTASSLD | constant region with leader |
| | YWGQGTLVTVSSASTTAPSVFPLAPSCGSTSG | |
| | STVALACLVSGYFPEPVTVSWNSGSLTSGVHT | |
| | FPSVLQSSGLYSLSSMVTVPSSRWPSETFTCN | |
| | VAHPASKTKVDKPVPKRENGRVPRPPDCPKCP | |
| | APEMLGGPSVFIFPPKPKDTLLIARTPEVTCV | |
| | VVDLDPEDPEVQISWFVDGKQMQTAKTQPREE | |
| | QFNGTYRVVSVLPIGHQDWLKGKQFTCKVNNK | |
| | ALPSPIERTISKARGQAHQPSVYVLPPSREEL | |
| | SKNTVSLTCLIKDFFPPDIDVEWQSNGQQEPE | |
| | SKYRTTPPQLDEDGSYFLYSKLSVDKSRWQRG | |
| | DTFICAVMHEALHNHYTQESLSHSPGK | |
| 22 | EVQLVESGGDLVKPGGSLRLSCVASGFTEDDY | Exemplary caninized |
| | AMHWVRQAPGKGLQWVSAITWNSGHIDYADAV | variable heavy chain of |
| | KGRFTISRDNAKNTLYLQMNSLRAEDTAVYYC | D2E7 |
| | AKVSYLSTASSLDYWGQGTLVTVSS | (KBA VH) |
| 23 | EVQLVESGGDLVKPGGSLRLSCVASGFTFDDY | Exemplary caninized |
| | AMHWVRQAPGKGLQWVSAITWNSGHIDYADAV | variable heavy chain of |
| | KGRFTISRDNAKNTLYLQMNSLRAEDTAVYYC | D2E7 and canine IgG-B |
| | AKVSYLSTASSLDYWGQGTLVTVSSASTTAPS | constant region |
| | VFPLAPSCGSTSGSTVALACLVSGYFPEPVTV | |
| | SWNSGSLTSGVHTFPSVLQSSGLYSLSSMVTV | |
| | PSSRWPSETFTCNVAHPASKTKVDKPVPKREN | |
| | GRVPRPPDCPKCPAPEMLGGPSVFIFPPKPKD | |
| | TLLIARTPEVTCVVVDLDPEDPEVQISWFVDG | |
| | KQMQTAKTQPREEQFNGTYRVVSVLPIGHQDW | |
| | LKGKQFTCKVNNKALPSPIERTISKARGQAHQ | |
| | PSVYVLPPSREELSKNTVSLTCLIKDFFPPDI | |
| | DVEWQSNGQQEPESKYRTTPPQLDEDGSYFLY | |
| | SKLSVDKSRWQRGDTFICAVMHEALHNHYTQE | |
| | SLSHSPGK | |
| 24 | MDMRVPAQLLGLLLLWLSGARCDIVMTQTPLS | Caninized variable light |
| | LSASPGEPVSISCRASQGIRNYLAWYQQKPGQ | chain of D2E7 v3 and |
| | APRLLIYAASTLQSGVPDRFSGSGSGTDFTLR | canine kappa light constant |
| | IGRLEAEDAAIYYCQRYNRAPYTFGQGTKVEI | region with leader |
| | KRNDAQPAVYLFQPSPDQLHTGSASVVCLLNS | |
| | FYPKDINVKWKVDGVIQDTGIQESVTEQDKDS | |
| | TYSLSSTLTMSSTEYLSHELYSCEITHKSLPS | |
| | TLIKSFQRSECQRVD | |
| 25 | DIVMTQTPLSLSASPGEPVSISCRASQGIRNY | Caninized variable light |
| | LAWYQQKPGQAPRLLIYAASTLQSGVPDRFSG | chain of D2E7 v3 |
| | SGSGTDFTLRIGRLEAEDAAIYYCQRYNRAPY | (KBA VL v3) |
| | TFGQGTKVEIK | |
| 26 | DIVMTQTPLSLSASPGEPVSISCRASQGIRNY | Caninized variable light |
| | LAWYQQKPGQAPRLLIYAASTLQSGVPDRFSG | chain of D2E7 v3 and |
| | SGSGTDFTLRIGRLEAEDAAIYYCQRYNRAPY | canine kappa light constant |
| | TFGQGTKVEIKRNDAQPAVYLFQPSPDQLHTG | region |
| | SASVVCLLNSFYPKDINVKWKVDGVIQDTGIQ | |
| | ESVTEQDKDSTYSLSSTLTMSSTEYLSHELYS | |
| | CEITHKSLPSTLIKSFQRSECQRVD | |
| 27 | WYQQKPGQAPRLLIY | Variable light chain |
| | | framework region LC-FR2 |
| | | of KBA VL v3 |
| 28 | MDMRVPAQLLGLLLLWLSGARCDIVMTQSPAS | Caninized variable light |
| | LSLSQEEKVTITCRASQGIRNYLAWYQQKPGQ | chain of D2E7 v4 and |
| | APKLLIYAASTLQSGVPSRFSGSGSGTDFSFT | canine kappa light constant |
| | ISSLEPEDVAVYYCQRYNRAPYTFGQGTKVEI | region with leader |
| | KRNDAQPAVYLFQPSPDQLHTGSASVVCLINS | |
| | FYPKDINVKWKVDGVIQDTGIQESVTEQDKDS | |
| | TYSLSSTLTMSSTEYLSHELYSCEITHKSLPS | |
| | TLIKSFQRSECQRVD | |
| 29 | DIVMTQSPASLSLSQEEKVTITCRASQGIRNY | Caninized variable light |
| | LAWYQQKPGQAPKLLIYAASTLQSGVPSRFSG | chain of D2E7 v4 |
| | SGSGTDFSFTISSLEPEDVAVYYCQRYNRAPY | (KBA VL v4) |
| | TFGQGTKVEIK | |
| 30 | DIVMTQSPASLSLSQEEKVTITCRASQGIRNY | Caninized variable light |
| | LAWYQQKPGQAPKLLIYAASTLQSGVPSRFSG | chain of D2E7 v4 and |
| | SGSGTDFSFTISSLEPEDVAVYYCQRYNRAPY | canine kappa light constant |
| | TFGQGTKVEIKRNDAQPAVYLFQPSPDQLHTG | region |
| | SASVVCLLNSFYPKDINVKWKVDGVIQDTGIQ | |
| | ESVTEQDKDSTYSLSSTLTMSSTEYLSHELYS | |
| | CEITHKSLPSTLIKSFQRSECQRVD | |
| 31 | WYQQKPGQAPKLLIY | Variable light chain |
| | | framework region LC-FR2 |
| | | of KBA VL v4 |
| 32 | MDMRVPAQLLGLLLLWLSGARCEIQMTQSPTL | Caninized variable light |
| | LSVSPGDRVTITCQASQGIRNYLAWYQQKRGN | chain of D2E7 v5 and |
| | APKCLIYAASTLQSGVPLRFSGSGSGTDFTLT | canine kappa light constant |
| | INSLEPKDVATYYCQRYNRAPYTFGQGTKVEI | region with leader |
| | KRNDAQPAVYLFQPSPDQLHTGSASVVCLLNS | |
| | FYPKDINVKWKVDGVIQDTGIQESVTEQDKDS | |
| | TYSLSSTLTMSSTEYLSHELYSCEITHKSLPS | |
| | TLIKSFQRSECQRVD | |
| 33 | EIQMTQSPTLLSVSPGDRVTITCQASQGIRNY | Caninized variable light |
| | LAWYQQKRGNAPKCLIYAASTLQSGVPLRFSG | chain of D2E7 v5 |
| | SGSGTDFTLTINSLEPKDVATYYCQRYNRAPY | (KBA VL v5) |
| | TFGQGTKVEIK | |
| 34 | EIQMTQSPTLLSVSPGDRVTITCQASQGIRNY | Caninized variable light |
| | LAWYQQKRGNAPKCLIYAASTLQSGVPLRFSG | chain of D2E7 v5 and |
| | SGSGTDFTLTINSLEPKDVATYYCQRYNRAPY | canine kappa light constant |
| | TFGQGTKVEIKRNDAQPAVYLFQPSPDQLHTG | region |
| | SASVVCLLNSFYPKDINVKWKVDGVIQDTGIQ | |
| | ESVTEQDKDSTYSLSSTLTMSSTEYLSHELYS | |
| | CEITHKSLPSTLIKSFQRSECQRVD | |
| 35 | WYQQKRGNAPKCLIY | Variable light chain |
| | | framework region LC-FR2 |
| | | of KBA VL v5 |
| 36 | MDMRVPAQLLGLLLLWLRGARCDIQMTQTPLS | Caninized variable light |
| | LSASPGEPVSISCRASQGIRNYLAWYLQKPGQ | chain of D2E7 v6 and |
| | SPRLLIYAASTLQSGVPDRFSGSGSGTDFTLR | canine kappa light constant |
| | IGRLEAEDAAIYYCQRYNRAPYTFGQGTKVEI | region with leader |
| | KRNDAQPAVYLFQPSPDQLHTGSASVVCLLNS | |
| | FYPKDINVKWKVDGVIQDTGIQESVTEQDKDS | |
| | TYSLSSTLTMSSTEYLSHELYSCEITHKSLPS | |
| | TLIKSFQRSECQRVD | |
| 37 | DIQMTQTPLSLSASPGEPVSISCRASQGIRNY | Caninized variable light |
| | LAWYLQKPGQSPRLLIYAASTLQSGVPDRFSG | chain of D2E7 v6 |
| | SGSGTDFTLRIGRLEAEDAAIYYCQRYNRAPY | (KBA VL v6) |
| | TFGQGTKVEIK | |
| 38 | DIQMTQTPLSLSASPGEPVSISCRASQGIRNY | Caninized variable light |
| | LAWYLQKPGQSPRLLIYAASTLQSGVPDRFSG | chain of D2E7 v6 and |
| | SGSGTDFTLRIGRLEAEDAAIYYCQRYNRAPY | canine kappa light constant |
| | TFGQGTKVEIKRNDAQPAVYLFQPSPDQLHTG | region |
| | SASVVCLLNSFYPKDINVKWKVDGVIQDTGIQ | |
| | ESVTEQDKDSTYSLSSTLTMSSTEYLSHELYS | |
| | CEITHKSLPSTLIKSFQRSECQRVD | |
| 39 | WYLQKPGQSPRLLIY | Variable light chain |
| | | framework region LC-FR2 |
| | | of KBA VL v6 |
| 40 | METDTLLLWVLLLWVPGSTGDIQMTQSPSSLS | Chimeric variable light |
| | ASVGDRVTITCRASQGIRNYLAWYQQKPGKAP | chain of D2E7 and canine |
| | KLLIYAASTLQSGVPSRFSGSGSGTDFTLTIS | lambda light constant |
| | SLQPEDVATYYCQRYNRAPYTFGQGTKVEIKR | region with leader |
| | TVAAPSVFIFPPSDEQLKSGTASVVCLLNNFY | |
| | PREAKVQWKVDNALQSGNSQESVTEQDSKDST | |
| | YSLSSTLTLSKADYEKHKVYACEVTHQGLSSP | |
| | VTKSFNRGEC | |
| 41 | DIQMTQSPSSLSASVGDRVTITCRASQGIRNY | Chimeric variable light |
| | LAWYQQKPGKAPKLLIYAASTLQSGVPSRFSG | chain of D2E7 and canine |
| | SGSGTDFTLTISSLQPEDVATYYCQRYNRAPY | lambda light constant |
| | TFGQGTKVEIKRTVAAPSVFIFPPSDEQLKSG | region |
| | TASVVCLLNNFYPREAKVQWKVDNALQSGNSQ | |
| | ESVTEQDSKDSTYSLSSTLTLSKADYEKHKVY | |
| | ACEVTHQGLSSPVTKSENRGEC | |
| 42 | METDTLLLWVLLLWVPGSTGDIVMTQSPASLS | Caninized variable light |
| | ASPEEKVTITCRASQGIRNYLAWYQQKPGKAP | chain of D2E7 v7 and |
| | KLLIYAASTLQSGVPSRFSGSGSGTDFTLTIS | canine kappa light constant |
| | SLEPEDVAVYYCQRYNRAPYTFGQGTKVEIKR | region with leader |
| | NDAQPAVYLFQPSPDQLHTGSASVVCLLNSFY | |
| | PKDINVKWKVDGVIQDTGIQESVTEQDKDSTY | |
| | SLSSTLTMSSTEYLSHELYSCEITHKSLPSTL | |
| | IKSFQRSECQRVD | |
| 43 | DIVMTQSPASLSASPEEKVTITCRASQGIRNY | Caninized variable light |
| | LAWYQQKPGKAPKLLIYAASTLQSGVPSRFSG | chain of D2E7 v7 |
| | SGSGTDFTLTISSLEPEDVAVYYCQRYNRAPY | (KBA VL v7) |
| | TFGQGTKVEIK | |
| 44 | DIVMTQSPASLSASPEEKVTITCRASQGIRNY | Caninized variable light |
| | LAWYQQKPGKAPKLLIYAASTLQSGVPSRFSG | chain of D2E7 v7 and |
| | SGSGTDFTLTISSLEPEDVAVYYCQRYNRAPY | canine kappa light constant |
| | TFGQGTKVEIKRNDAQPAVYLFQPSPDQLHTG | region |
| | SASVVCLLNSFYPKDINVKWKVDGVIQDTGIQ | |
| | ESVTEQDKDSTYSLSSTLTMSSTEYLSHELYS | |
| | CEITHKSLPSTLIKSFQRSECQRVD | |
| 45 | WYQQKPGKAPKLLIY | Variable light chain |
| | | framework region LC-FR2 |
| | | of KBA VL v7 |
| 46 | DIVMTQTPLSLSVSPGEPASISCRASQGIRNY | Caninized variable light |
| | LAWYQQKPGKAPKLLIYAASTLQSGVPDRFSG | chain of D2E7 v8 |
| | SGSGTDFTLRIGRVEAEDAGIYYCQRYNRAPY | |
| | TFGQGTRLEVR | |
| 47 | DILLTQSPAILSVSPGERVSFSCRASQFVGSS | Variable light chain of |
| | IHWYQQRTNGSPRLLIKYASESMSGIPSRFSG | mouse clone cA2 |
| | SGSGTDFTLSINTVESEDIADYYCQQSHSWPF | (Infliximab) |
| | TFGSGTNLEVK | |
| 48 | EVKLEESGGGLVQPGGSMKLSCVASGFIFSNH | Variable heavy chain of |
| | WMNWVRQSPEKGLEWVAEIRSKSINSATHYAE | mouse clone cA2 |
| | SVKGRFTISRDDSKSAVYLQMTDLRTEDTGVY | (Infliximab) |
| | YCSRNYYGSTYDYWGQGTTLTVSS | |
| 49 | RASQFVGSSIH | Variable light chain |
| | | CDR-L1 of cA2 |
| 50 | YASESMS | Variable light chain |
| | | CDR-L2 of cA2 |
| 51 | QQSHSWPFT | Variable light chain |
| | | CDR-L3 of cA2 |
| 52 | NHWMN | Variable heavy chain |
| | | CDR-H1 of cA2 |
| 53 | EIRSKSINSATHYAESVKG | Variable heavy chain |
| | | CDR-H2 of cA2 |
| 54 | NYYGSTYDY | Variable heavy chain |
| | | CDR-H3 of cA2 |
| 55 | DIVMTQTPLSLSVSPGETASISCRASQFVGSS | Caninized variable light |
| | IHWFRQKPGQSPQRLIKYASESMSGVPDRFSG | chain of cA2 v1 |
| | SGSGTDFTLRINTVEADDTGVYYCQQSHSWPF | |
| | TFGSGTKLEIK | |
| 56 | DIVMTQTPLSLSVSPGETVSFSCRASQFVGSS | Caninized variable light |
| | IHWFRQKPGQSPQLLIKYASESMSGIPDRFSG | chain of cA2 v2 |
| | SGSGTDFTLSINTVEADDIAVYYCQQSHSWPF | |
| | TFGSGTKLEIK | |
| 57 | EVQLVESGGDLVKPGGSLRLSCVASGFIFSNH | Caninized variable heavy |
| | WMNWVRQAPGKGLQWVAEIRSKSINSATHYAE | chain of mouse clone cA2 |
| | SVKGRFTISRDNARNTLYLQMNSLRSEDTGVY | v1 |
| | YCSRNYYGSTYDYWGQGTTVTVSS | |
| 58 | EVQLVESGGGLVKPGGSLRLSCVASGFIFSNH | Caninized variable heavy |
| | WMNWVRQSPGKGLQWVAEIRSKSINSATHYAE | chain of mouse clone cA2 |
| | SVKGRFTISRDNARNTVYLQMNSLRSEDTGVY | v2 |
| | YCSRNYYGSTYDYWGQGTTVTVSS | |
| 59 | DIVMTQTPLSLSVSPGETASISCRASQFVGSS | Caninized variable light |
| | IHWFRQKPGQSPQRLIKYASESMSGVPDRFSG | chain of cA2 v1 and canine |
| | SGSGTDFTLRINTVEADDTGVYYCQQSHSWPF | kappa light constant region |
| | TFGSGTKLEIKRNDAQPAVYLFQPSPDQLHTG | |
| | SASVVCLLNSFYPKDINVKWKVDGVIQDTGIQ | |
| | ESVTEQDKDSTYSLSSTLTMSSTEYLSHELYS | |
| | CEITHKSLPSTLIKSFQRSECQRVD | |
| 60 | DIVMTQTPLSLSVSPGETVSFSCRASQFVGSS | Caninized variable light |
| | IHWFRQKPGQSPQLLIKYASESMSGIPDRFSG | chain of cA2 v2 and canine |
| | SGSGTDFTLSINTVEADDIAVYYCQQSHSWPF | kappa light constant region |
| | TFGSGTKLEIKRNDAQPAVYLFQPSPDQLHTG | |
| | SASVVCLLNSFYPKDINVKWKVDGVIQDTGIQ | |
| | ESVTEQDKDSTYSLSSTLTMSSTEYLSHELYS | |
| | CEITHKSLPSTLIKSFQRSECQRVD | |
| 61 | EVQLVESGGDLVKPGGSLRLSCVASGFIFSNH | Caninized variable heavy |
| | WMNWVRQAPGKGLQWVAEIRSKSINSATHYAE | chain of cA2 v1 and canine |
| | SVKGRFTISRDNARNTLYLQMNSLRSEDTGVY | IgG-B constant region |
| | YCSRNYYGSTYDYWGQGTTVTVSSASTTAPSV | |
| | FPLAPSCGSTSGSTVALACLVSGYFPEPVTVS | |
| | WNSGSLTSGVHTFPSVLQSSGLYSLSSMVTVP | |
| | SSRWPSETFTCNVAHPASKTKVDKPVPKRENG | |
| | RVPRPPDCPKCPAPEMLGGPSVFIFPPKPKDT | |
| | LLIARTPEVTCVVVDLDPEDPEVQISWFVDGK | |
| | QMQTAKTQPREEQFNGTYRVVSVLPIGHQDWL | |
| | KGKQFTCKVNNKALPSPIERTISKARGQAHQP | |
| | SVYVLPPSREELSKNTVSLTCLIKDFFPPDID | |
| | VEWQSNGQQEPESKYRTTPPQLDEDGSYFLYS | |
| | KLSVDKSRWQRGDTFICAVMHEALHNHYTQES | |
| | LSHSPGK | |
| 62 | EVQLVESGGGLVKPGGSLRLSCVASGFIFSNH | Caninized variable heavy |
| | WMNWVRQSPGKGLQWVAEIRSKSINSATHYAE | chain of cA2 v2 and canine |
| | SVKGRFTISRDNARNTVYLQMNSLRSEDTGVY | IgG-B constant region |
| | YCSRNYYGSTYDYWGQGTTVTVSSASTTAPSV | |
| | FPLAPSCGSTSGSTVALACLVSGYFPEPVTVS | |
| | WNSGSLTSGVHTFPSVLQSSGLYSLSSMVTVP | |
| | SSRWPSETFTCNVAHPASKTKVDKPVPKRENG | |
| | RVPRPPDCPKCPAPEMLGGPSVFIFPPKPKDT | |
| | LLIARTPEVTCVVVDLDPEDPEVQISWFVDGK | |
| | QMQTAKTQPREEQFNGTYRVVSVLPIGHQDWL | |
| | KGKQFTCKVNNKALPSPIERTISKARGQAHQP | |
| | SVYVLPPSREELSKNTVSLTCLIKDFFPPDID | |
| | VEWQSNGQQEPESKYRTTPPQLDEDGSYFLYS | |
| | KLSVDKSRWQRGDTFICAVMHEALHNHYTQES | |
| | LSHSPGK | |
| 63 | MGWSWIFLFLLSGTAGVHSEVQLVESGGDLVK | Exemplary caninized |
| | PGGSLRLSCVASGFTFDDYAMHWVRQAPGKGL | variable heavy chain of |
| | QWVSAITWNSGHIDYADAVKGRFTISRDNAKN | D2E7 and variant canine |
| | TLYLQMNSLRAEDTAVYYCAKVSYLSTASSLD | IgG-B (FcRn long-acting |
| | YWGQGTLVTVSSASTTAPSVFPLAPSCGSTSG | Q to Y) constant region |
| | STVALACLVSGYFPEPVTVSWNSGSLTSGVHT | with leader |
| | FPSVLQSSGLYSLSSMVTVPSSRWPSETFTCN | |
| | VAHPASKTKVDKPVPKRENGRVPRPPDCPKCP | |
| | APEMLGGPSVFIFPPKPKDTLLIARTPEVTCV | |
| | VVDLDPEDPEVQISWFVDGKQMQTAKTQPREE | |
| | QFNGTYRVVSVLPIGHYDWLKGKQFTCKVNNK | |
| | ALPSPIERTISKARGQAHQPSVYVLPPSREEL | |
| | SKNTVSLTCLIKDFFPPDIDVEWQSNGQQEPE | |
| | SKYRTTPPQLDEDGSYFLYSKLSVDKSRWQRG | |
| | DTFICAVMHEALHNHYTQESLSHSPGK | |
| 64 | EVQLVESGGDLVKPGGSLRLSCVASGFTEDDY | Exemplary caninized |
| | AMHWVRQAPGKGLQWVSAITWNSGHIDYADAV | variable heavy chain of |
| | KGRFTISRDNAKNTLYLQMNSLRAEDTAVYYC | D2E7 and variant canine |
| | AKVSYLSTASSLDYWGQGTLVTVSSASTTAPS | IgG-B (FcRn long-acting |
| | VFPLAPSCGSTSGSTVALACLVSGYFPEPVTV | Q to Y) constant region |
| | SWNSGSLTSGVHTFPSVLQSSGLYSLSSMVTV | |
| | PSSRWPSETFTCNVAHPASKTKVDKPVPKREN | |
| | GRVPRPPDCPKCPAPEMLGGPSVFIFPPKPKD | |
| | TLLIARTPEVTCVVVDLDPEDPEVQISWFVDG | |
| | KQMQTAKTQPREEQFNGTYRVVSVLPIGHYDW | |
| | LKGKQFTCKVNNKALPSPIERTISKARGQAHQ | |
| | PSVYVLPPSREELSKNTVSLTCLIKDFFPPDI | |
| | DVEWQSNGQQEPESKYRTTPPQLDEDGSYFLY | |
| | SKLSVDKSRWQRGDTFICAVMHEALHNHYTQE | |
| | SLSHSPGK | |
| 65 | RASEDIYNALA | Variable light chain |
| | | CDR-L1 of αD11 |
| 66 | YNTDTLHT | Variable light chain |
| | | CDR-L2 of αD11 |
| 67 | QHYFGYPRT | Variable light chain |
| | | CDR-L3 of αD11 |
| 68 | TVSGFSLTNNNVN | Variable heavy chain |
| | | CDR-H1 of αD11 |
| 69 | GVWAGGATD | Variable heavy chain |
| | | CDR-H2 of αD11 |
| 70 | ARDGGYSSSTLYAMDA | Variable heavy chain |
| | | CDR-H3 of αD11 |
| 71 | DIQMTQSPASLSASLGETVTIECRASEDIYNALAW | Variable light chain of rat |
| | YQQKPGKSPQLLIYNTDTLHTGVPSRFSGSGSGTQ | αD11 |
| | YSLKINSLQSEDVASYFCQHYFGYPRTFGGGTKLE | |
| | LK | |
| 72 | QVQLKESGPGLVQPSQTLSLTCTVSGFSLTNNNVN | Variable heavy chain of rat |
| | WVRQATGRGLEWMGGVWAGGATDYNSALKSRLTIT | αD11 |
| | RDTSKSQVFLKMHSLQSEDTATYYCARDGGYSSST | |
| | LYAMDAWGQGTTVTVSS | |
| 73 | EIVMTQSPGSLAGSAGESVSINCRASEDIYNALAW | Exemplary caninized |
| | YQQKPGERPKLLIYNTDTLHTGVPARFSSSGSGTD | variable light chain of |
| | FTLTINNLQAEDVGDYFCQHYFGYPRTFGAGTKVE | αD11 |
| | LK | |
| 74 | EVQLVESGGDLVKPAGSLRLSCVASGFSLTNNNVN | Exemplary caninized |
| | WVRQAPGKGLEWMGGVWAGGATDYNSALKSRFTIS | variable heavy chain of |
| | RDNAKNTLYLQMNSLRAEDTAVYYCARDGGYSSST | αD11 |
| | LYAMDAWGQGTLVTVSS | |
| 75 | VASGFSLINNNVN | Variable heavy chain |
| | | CDR-H1 of caninized |
| | | αD11 |
| 76 | METDTLLLWVLLLWVPGSTGEIVMTQSPGSLAGSA | Exemplary caninized |
| | GESVSINCRASEDIYNALAWYQQKPGERPKLLIYN | variable light chain of |
| | TDTLHTGVPARFSSSGSGTDFTLTINNLQAEDVGD | αD11 and canine kappa |
| | YFCQHYFGYPRTFGAGTKVELKRNDAQPAVYLFQP | light constant region with |
| | SPDQLHTGSASVVCLLNSFYPKDINVKWKVDGVIQ | leader |
| | DTGIQESVTEQDKDSTYSLSSTLTMSSTEYLSHEL | |
| | YSCEITHKSLPSTLIKSFQRSECQRVD | |
| 77 | EIVMTQSPGSLAGSAGESVSINCRASEDIYNALAW | Exemplary caninized |
| | YQQKPGERPKLLIYNTDTLHTGVPARFSSSGSGTD | variable light chain of |
| | FTLTINNLQAEDVGDYFCQHYFGYPRTFGAGTKVE | αD11 and canine kappa |
| | LKRNDAQPAVYLFQPSPDQLHTGSASVVCLINSFY | light constant region |
| | PKDINVKWKVDGVIQDTGIQESVTEQDKDSTYSLS | |
| | STLTMSSTEYLSHELYSCEITHKSLPSTLIKSFQR | |
| | SECQRVD | |
| 78 | MAVLGLLLCLVTFPSCVLSEVQLVESGGDLVK | Exemplary caninized |
| | PAGSLRLSCVASGFSLTNNNVNWVRQAPGKGL | variable heavy chain of |
| | EWMGGVWAGGATDYNSALKSRFTISRDNAKNT | αD11 and variant canine |
| | LYLQMNSLRAEDTAVYYCARDGGYSSSTLYAM | IgG-B (FcRn long-acting |
| | DAWGQGTLVTVSSASTTAPSVFPLAPSCGSTS | Q to Y, C1q-, CD16-) |
| | GSTVALACLVSGYFPEPVTVSWNSGSLTSGVH | constant region with leader |
| | TFPSVLQSSGLYSLSSMVTVPSSRWPSETFTC | |
| | NVAHPASKTKVDKPVPKRENGRVPRPPDCPKC | |
| | PAPEMLGGPSVFIFPPKPKDTLLIARTPEVTC | |
| | VVVDLDPEDPEVQISWFVDGKQMQTAKTQPRE | |
| | EQFNGTYRVVSVLPIGHYDWLKGKQFTCRVNN | |
| | IGLPSPIERTISKARGQAHQPSVYVLPPSREE | |
| | LSKNTVSLTCLIKDFFPPDIDVEWQSNGQQEP | |
| | ESKYRTTPPQLDEDGSYFLYSKLSVDKSRWQR | |
| | GDTFICAVMHEALHNHYTQESLSHSPGK | |
| 79 | EVQLVESGGDLVKPAGSLRLSCVASGFSLINN | Exemplary caninized |
| | NVNWVRQAPGKGLEWMGGVWAGGATDYNSALK | variable heavy chain of |
| | SRFTISRDNAKNTLYLQMNSLRAEDTAVYYCA | αD11 and variant canine |
| | RDGGYSSSTLYAMDAWGQGTLVTVSSASTTAP | IgG-B (FcRn long-acting |
| | SVFPLAPSCGSTSGSTVALACLVSGYFPEPVT | Q to Y, C1q-, CD16-) |
| | VSWNSGSLTSGVHTFPSVLQSSGLYSLSSMVT | constant region |
| | VPSSRWPSETFTCNVAHPASKTKVDKPVPKRE | |
| | NGRVPRPPDCPKCPAPEMLGGPSVFIFPPKPK | |
| | DTLLIARTPEVTCVVVDLDPEDPEVQISWFVD | |
| | GKQMQTAKTQPREEQFNGTYRVVSVLPIGHYD | |
| | WLKGKQFTCRVNNIGLPSPIERTISKARGQAH | |
| | QPSVYVLPPSREELSKNTVSLTCLIKDFFPPD | |
| | IDVEWQSNGQQEPESKYRTTPPQLDEDGSYFL | |
| | YSKLSVDKSRWQRGDTFICAVMHEALHNHYTQ | |
| | ESLSHSPGK | |
| 80 | MAVLGLLLCLVTFPSCVLSEVQLVESGGDLVKPAG | Exemplary bi-specific |
| | SLRLSCVASGFSLTNNNVNWVRQAPGKGLEWMGGV | NGF and TNF antibody |
| | WAGGATDYNSALKSRFTISRDNAKNTLYLQMNSLR | with leader |
| | AEDTAVYYCARDGGYSSSTLYAMDAWGQGTLVTVS | (Anti-NGF + Fc IgGB |
| | SASTTAPSVFPLAPSCGSTSGSTVALACLVSGYFP | (FcRn long-acting Q to Y, |
| | EPVTVSWNSGSLTSGVHTFPSVLQSSGLYSLSSMV | C1q-, CD16-) + anti-TNF |
| | TVPSSRWPSETFTCNVAHPASKTKVDKPVPKRENG | ScFv) |
| | RVPRPPDCPKCPAPEMLGGPSVFIFPPKPKDTLLI | |
| | ARTPEVTCVVVDLDPEDPEVQISWFVDGKQMQTAK | |
| | TQPREEQFNGTYRVVSVLPIGHYDWLKGKQFTCRV | |
| | NNIGLPSPIERTISKARGQAHQPSVYVLPPSREEL | |
| | SKNTVSLTCLIKDFFPPDIDVEWQSNGQQEPESKY | |
| | RTTPPQLDEDGSYFLYSKLSVDKSRWQRGDTFICA | |
| | VMHEALHNHYTQESLSHSPGKGGGGSGGGEVQLVE | |
| | SGGDLVKPGGSLRLSCVASGFTFDDYAMHWVRQAP | |
| | GKGLQWVSAITWNSGHIDYADAVKGRFTISRDNAK | |
| | NTLYLQMNSLRAEDTAVYYCAKVSYLSTASSLDYW | |
| | GQGTLVTVSSGGGGGSGGGGSGGGGGSDIVMTQSP | |
| | ASLSASPEEKVTITCRASQGIRNYLAWYQQKPGKA | |
| | PKLLIYAASTLQSGVPSRFSGSGSGTDFTLTISSL | |
| | EPEDVAVYYCQRYNRAPYTFGQGTKVEIK | |
| 81 | EVQLVESGGDLVKPAGSLRLSCVASGFSLINNNVN | Exemplary bi-specific |
| | WVRQAPGKGLEWMGGVWAGGATDYNSALKSRFTIS | NGF and TNF antibody |
| | RDNAKNTLYLQMNSLRAEDTAVYYCARDGGYSSST | (Anti-NGF + Fc IgGB |
| | LYAMDAWGQGTLVTVSSASTTAPSVFPLAPSCGST | (FcRn long-acting Q to Y, |
| | SGSTVALACLVSGYFPEPVTVSWNSGSLTSGVHTF | C1q-, CD16-) + anti-TNF |
| | PSVLQSSGLYSLSSMVTVPSSRWPSETFTCNVAHP | ScFv) |
| | ASKTKVDKPVPKRENGRVPRPPDCPKCPAPEMLGG | |
| | PSVFIFPPKPKDTLLIARTPEVTCVVVDLDPEDPE | |
| | VQISWFVDGKQMQTAKTQPREEQFNGTYRVVSVLP | |
| | IGHYDWLKGKQFTCRVNNIGLPSPIERTISKARGQ | |
| | AHQPSVYVLPPSREELSKNTVSLTCLIKDFFPPDI | |
| | DVEWQSNGQQEPESKYRTTPPQLDEDGSYFLYSKL | |
| | SVDKSRWQRGDTFICAVMHEALHNHYTQESLSHSP | |
| | GKGGGGSGGGEVQLVESGGDLVKPGGSLRLSCVAS | |
| | GFTFDDYAMHWVRQAPGKGLQWVSAITWNSGHIDY | |
| | ADAVKGRFTISRDNAKNTLYLQMNSLRAEDTAVYY | |
| | CAKVSYLSTASSLDYWGQGTLVTVSSGGGGGSGGG | |
| | GSGGGGGSDIVMTQSPASLSASPEEKVTITCRASQ | |
| | GIRNYLAWYQQKPGKAPKLLIYAASTLQSGVPSRF | |
| | SGSGSGTDFTLTISSLEPEDVAVYYCQRYNRAPYT | |
| | FGQGTKVEIK | |
| 82 | MAVLGLLLCLVTFPSCVLSEVQLVESGGDLVKPGG | Exemplary bi-specific |
| | SLRLSCVASGFTFDDYAMHWVRQAPGKGLQWVSAI | NGF and TNF antibody |
| | TWNSGHIDYADAVKGRFTISRDNAKNTLYLQMNSL | with leader |
| | RAEDTAVYYCAKVSYLSTASSLDYWGQGTLVTVSS | (Anti-TNF + Fc IgGB |
| | ASTTAPSVFPLAPSCGSTSGSTVALACLVSGYFPE | (FcRn long-acting Q to Y, |
| | PVTVSWNSGSLTSGVHTFPSVLQSSGLYSLSSMVT | C1q-, CD16-) + anti-NGF |
| | VPSSRWPSETFTCNVAHPASKTKVDKPVPKRENGR | ScFv) |
| | VPRPPDCPKCPAPEMLGGPSVFIFPPKPKDTLLIA | |
| | RTPEVTCVVVDLDPEDPEVQISWFVDGKQMQTAKT | |
| | QPREEQFNGTYRVVSVLPIGHYDWLKGKQFTCRVN | |
| | NIGLPSPIERTISKARGQAHQPSVYVLPPSREELS | |
| | KNTVSLTCLIKDFFPPDIDVEWQSNGQQEPESKYR | |
| | TTPPQLDEDGSYFLYSKLSVDKSRWQRGDTFICAV | |
| | MHEALHNHYTQESLSHSPGKGGGGSGGGEVQLVES | |
| | GGDLVKPAGSLRLSCVASGFSLINNNVNWVRQAPG | |
| | KGLEWMGGVWAGGATDYNSALKSRFTISRDNAKNT | |
| | LYLQMNSLRAEDTAVYYCARDGGYSSSTLYAMDAW | |
| | GQGTLVTVSSGGGGGSGGGGSGGGGGSEIVMTQSP | |
| | GSLAGSAGESVSINCRASEDIYNALAWYQQKPGER | |
| | PKLLIYNTDTLHTGVPARFSSSGSGTDFTLTINNL | |
| | QAEDVGDYFCQHYFGYPRTFGAGTKVELK | |
| 83 | EVQLVESGGDLVKPGGSLRLSCVASGFTEDDYAMH | Exemplary bi-specific |
| | WVRQAPGKGLQWVSAITWNSGHIDYADAVKGRFTI | NGF and TNF antibody |
| | SRDNAKNTLYLQMNSLRAEDTAVYYCAKVSYLSTA | (Anti-TNF + Fc IgGB |
| | SSLDYWGQGTLVTVSSASTTAPSVFPLAPSCGSTS | (FcRn long-acting Q to Y, |
| | GSTVALACLVSGYFPEPVTVSWNSGSLTSGVHTFP | C1q-, CD16-) + anti-NGF |
| | SVLQSSGLYSLSSMVTVPSSRWPSETFTCNVAHPA | ScFv) |
| | SKTKVDKPVPKRENGRVPRPPDCPKCPAPEMLGGP | |
| | SVFIFPPKPKDTLLIARTPEVTCVVVDLDPEDPEV | |
| | QISWFVDGKQMQTAKTQPREEQFNGTYRVVSVLPI | |
| | GHYDWLKGKQFTCRVNNIGLPSPIERTISKARGQA | |
| | HQPSVYVLPPSREELSKNTVSLTCLIKDFFPPDID | |
| | VEWQSNGQQEPESKYRTTPPQLDEDGSYFLYSKLS | |
| | VDKSRWQRGDTFICAVMHEALHNHYTQESLSHSPG | |
| | KGGGGSGGGEVQLVESGGDLVKPAGSLRLSCVASG | |
| | FSLTNNNVNWVRQAPGKGLEWMGGVWAGGATDYNS | |
| | ALKSRFTISRDNAKNTLYLQMNSLRAEDTAVYYCA | |
| | RDGGYSSSTLYAMDAWGQGTLVTVSSGGGGGSGGG | |
| | GSGGGGGSEIVMTQSPGSLAGSAGESVSINCRASE | |
| | DIYNALAWYQQKPGERPKLLIYNTDTLHTGVPARF | |
| | SSSGSGTDFTLTINNLQAEDVGDYFCQHYFGYPRT | |
| | FGAGTKVELK | |

### Description of the Embodiments

Antibodies that bind canine TNFα and/or canine NGF are provided. Antibody heavy chains and light chains that are capable of forming antibodies that bind canine TNFα and/or canine NGF are also provided. In addition, antibodies, heavy chains, and light chains comprising one or more particular complementary determining regions (CDRs) are provided. Polynucleotides encoding antibodies to canine TNFα and/or canine NGF are provided. Methods of producing or purifying antibodies to canine TNFα and/or canine NGF are also provided. Methods of treatment using antibodies to canine TNFα and/or canine NGF are provided. Such methods include, but are not limited to, methods of conditions associated with TNFα and/or NGF in canines. Methods of detecting TNFα and/or NGF in a sample from a companion animal species are provided.

For the convenience of the reader, the following definitions of terms used herein are provided.

As used herein, numerical terms such as Kd are calculated based upon scientific measurements and, thus, are subject to appropriate measurement error. In some instances, a numerical term may include numerical values that are rounded to the nearest significant figure.

As used herein, “a” or “an” means “at least one” or “one or more” unless otherwise specified. As used herein, the term “or” means “and/or” unless specified otherwise. In the context of a multiple dependent claim, the use of “or” when referring back to other claims refers to those claims in the alternative only.

Exemplary TNFα and/or NGF Antibodies

Novel antibodies directed against TNFα and/or NGF are provided, for example antibodies that bind to canine TNFα and or canine NGF. TNFα and/or NGF antibodies provided herein include, but are not limited to, monoclonal antibodies, chimeric antibodies, and caninized antibodies. In some embodiments, a TNFα antibody is KIND-509.

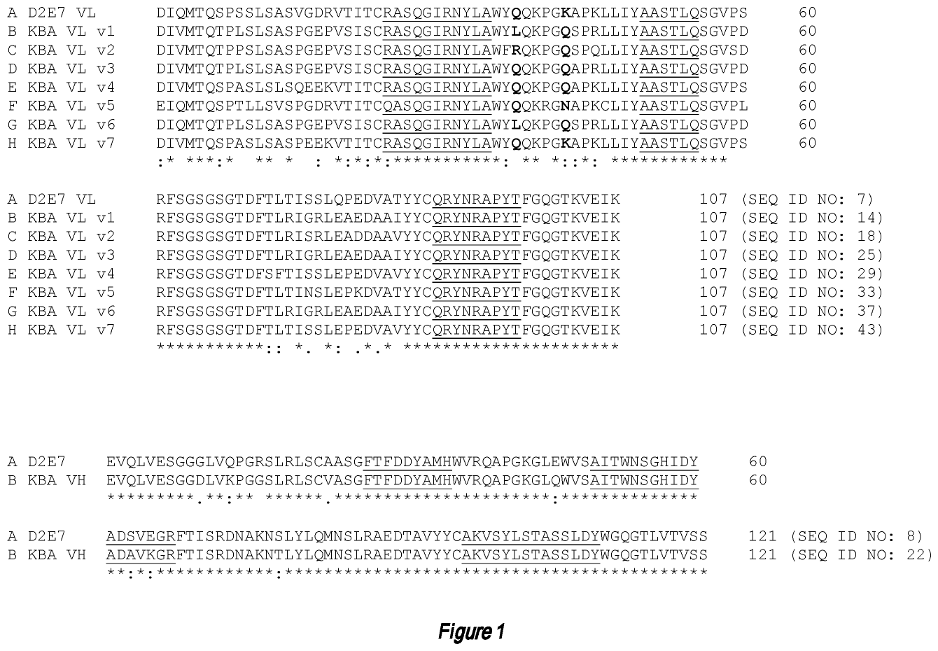

Also provided herein are amino acid sequences of monoclonal antibodies. For example, the variable heavy chain CDRs, variable light chain CDRs, variable region heavy chain framework sequences, and variable region light chain framework sequences for monoclonal antibodies described herein are provided. For example, amino acid sequences of the variable light chain and variable heavy chain of monoclonal antibody KIND-509 are provided (SEQ ID NOs: 14 and 22, respectively). In addition, the amino acid sequences of CDRs, framework sequences, variable light chain sequences, and variable heavy chain sequences of different caninized light and heavy chains are provided in FIG. 1.

Also provided herein are chimeric antibodies derived from monoclonal antibody D2E7. In some embodiments, amino acid sequences of caninized monoclonal antibody D2E7 are provided, such as SEQ ID NOs: 13-46, 55-64, and 80-83. In some embodiments, amino acid sequences of chimeric antibodies derived from monoclonal antibody D2E7 are provided, such as SEQ ID NOs: 9-12.

Also provided herein are chimeric antibodies derived from monoclonal antibody αD11. In some embodiments, amino acid sequences of caninized monoclonal antibody αD11 are provided, such as SEQ ID NOs: 73-83.

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments (such as Fab, F(ab′) 2, ScFv, minibody, diabody, triabody, and tetrabody) so long as they exhibit the desired antigen-binding activity. Canine, feline, and equine species have different varieties (classes) of antibodies that are shared by many mammalians

The term antibody includes, but is not limited to, fragments that are capable of binding to an antigen, such as Fv, single-chain Fv (scFv), Fab, Fab′, di-scFv, sdAb (single domain antibody) and (Fab′)2 (including a chemically linked F(ab′)2). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen combining sites and is still capable of cross-linking antigen. The term antibody also includes, but is not limited to, chimeric antibodies, humanized antibodies, and antibodies of various species such as mouse, human, cynomolgus monkey, canine, feline, equine, etc. Furthermore, for all antibody constructs provided herein, variants having the sequences from other organisms are also contemplated. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, mouse scFv or a canine scFv). This denotes the sequences of at least part of the non-CDR regions, rather than the source of the construct. In some embodiments, the antibodies comprise a label or are conjugated to a second moiety.

The terms “label” and “detectable label” mean a moiety attached to an antibody or its analyte to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵s, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

The term “monoclonal antibody” refers to an antibody of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to poly clonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

In some embodiments, the monoclonal antibody is KIND-509.

“Amino acid sequence,” means a sequence of amino acids residues in a peptide or protein. The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“TNFα” as used herein refers to any native TNFα that results from expression and processing of TNFα in a cell. The term includes TNFα from any vertebrate source, including mammals such as primates (e.g., humans and cynomolgus monkeys) and rodents (e.g., mice and rats), and companion animals (e.g., dogs, cats, and equine), unless otherwise indicated. The term also includes naturally occurring variants of TNFα, e.g., splice variants or allelic variants.

“NGF” or “nerve growth factor” as used herein refers to any NGF that results from expression and processing of NGF in a cell. The term includes NGF from any vertebrate source, including mammals such as primates (e.g., humans and cynomolgus monkeys) and rodents (e.g., mice and rats), and companion animals (e.g., dogs, cats, and equine), unless otherwise indicated. The term also includes naturally occurring variants of NGF, e.g., splice variants or allelic variants.

As used herein, the term “epitope” refers to a site on a target molecule (for example, an antigen, such as a protein, nucleic acid, carbohydrate or lipid) to which an antigen-binding molecule (for example, an antibody, antibody fragment, or scaffold protein containing antibody binding regions) binds. Epitopes often include a chemically active surface grouping of molecules such as amino acids, polypeptides or sugar side chains and have specific three dimensional structural characteristics as well as specific charge characteristics. Epitopes can be formed both from contiguous or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some examples an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between an antibody residue and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antibody. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

The term “CDR” means a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the contact definition, or a combination of the Kabat, Chothia, AbM, or contact definitions. The various CDRs within an antibody can be designated by their appropriate number and chain type, including, without limitation as CDR-H1, CDR-H2, CDR-H3, CDR-L1, CDR-L2, and CDR-L3. The term “CDR” is used herein to also encompass a “hypervariable region” or HVR, including hypervariable loops.

In some embodiments, a caninized TNFα antibody comprises a variable light chain comprising (a) a CDR-L1 comprising the amino acid sequence of SEQ ID NO: 1; (b) a CDR-L2 comprising the amino acid sequence of SEQ ID NO: 2; and/or (c) a CDR-L3 comprising the amino acid sequence of SEQ ID NO: 3. In some embodiments, the caninized TNFαantibody comprises a variable light chain comprising a LC-FR2 comprising a glutamine at position 3 (corresponding to KABAT position 37) and a lysine at position 8 (corresponding to KABAT position 42. In some embodiments, the caninized TNFα antibody comprises a variable heavy chain comprising (a) a CDR-H1 comprising the amino acid sequence of SEQ ID NO: 4; (b) a CDR-H2 comprising the amino acid sequence of SEQ ID NO: 5; and/or (c) a CDR-H3 comprising the amino acid sequence of SEQ ID NO: 6.

In some embodiments, a caninized TNFα antibody comprises a variable light chain comprising (a) a CDR-L1 comprising the amino acid sequence of SEQ ID NO: 49; (b) a CDR-L2 comprising the amino acid sequence of SEQ ID NO: 50; and/or (c) a CDR-L3 comprising the amino acid sequence of SEQ ID NO: 51. In some embodiments, a caninized TNFαantibody comprises a variable heavy chain comprising (a) a CDR-H1 comprising the amino acid sequence of SEQ ID NO: 52; (b) a CDR-H2 comprising the amino acid sequence of SEQ ID NO: 53; and/or (c) a CDR-H3 comprising the amino acid sequence of SEQ ID NO: 54.

In some embodiments, a caninized NGF antibody comprises (i) a CDR-H1 comprising the amino acid sequence of SEQ ID NO: 75; (ii) a CDR-H2 comprising the amino acid sequence of SEQ ID NO: 69, and/or (iii) a CDR-H3 comprising the amino acid sequence of SEQ ID NO: 70. In some embodiments, a caninized NGF antibody (i) a CDR-L1 comprising the amino acid sequence of SEQ ID NO: 65; (ii) a CDR-L2 comprising the amino acid sequence of SEQ ID NO: 66; and/or (iii) a CDR L3 comprising the amino acid sequence of SEQ ID NO: 67.

The term “variable region” as used herein refers to a region comprising at least three CDRs. In some embodiments, the variable region includes the three CDRs and at least one framework region (“FR”). The terms “heavy chain variable region” or “variable heavy chain” are used interchangeably to refer to a region comprising at least three heavy chain CDRs. The terms “light chain variable region” or “variable light chain” are used interchangeably to refer to a region comprising at least three light chain CDRs. In some embodiments, the variable heavy chain or variable light chain comprises at least one framework region. In some embodiments, an antibody comprises at least one light chain framework region selected from LC-FR1, LC-FR2, LC-FR3, and LC-FR4. In some embodiments, an antibody comprises at least one heavy chain framework region selected from HC-FR1, HC-FR2, HC-FR3, and HC-FR4. The framework regions may be juxtaposed between light chain CDRs or between heavy chain CDRs. For example, an antibody may comprise a variable light chain having the following structure: (LC-1-R1)-(CDR-L1)-(LC-FR2)-(CDR-L2)-(LC-FR3)-(CDR-L3)-(LC-FR4). An antibody may also comprise a variable light chain having the following structure: (CDR-L1)-(LC-FR2)-(CDR-L2)-(LC-FR3)-(CDR-L3). An antibody may also comprise a variable heavy chain having the following structure: (HC-FR1)-(CDR-H1)-(HC-FR2)-(CDR-H2)-(HC-1-R3)-(CDR-H3)-(HC-PR4). An antibody may comprise a variable heavy chain having the following structure: (CDR-H1)-(HC-FR2)-(CDR-H2)-(HC-FR3)-(CDR-H3).

In some embodiments, a TNFα antibody comprises an LC-FR2 sequence of SEQ ID NO: 16, SEQ ID NO: 20, SEQ ID NO: 27, SEQ ID NO: 31, SEQ ID NO: 35, SEQ ID NO: 39, or SEQ ID NO: 45.

In some embodiments, the TNFα antibody comprises (i) a variable light chain comprising the amino acid sequence of SEQ ID NO: 43 or SEQ ID NO: 46 or a variant thereof wherein 1, 2, 3, 4, 5, or 6 amino acids of the variable light chain is substituted by a different amino acid, (ii) a variable heavy chain comprising the amino acid sequence of SEQ ID NO: 22 or a variant thereof wherein 1, 2, 3, 4, 5, or 6 amino acids of the variable heavy chain is substituted by a different amino acid, or (iii) a variable light chain as in (i) and a variable heavy chain as in (ii).

In some embodiments, the TNFα antibody comprises (i) a variable light chain comprising the amino acid sequence of SEQ ID NO: 43 or SEQ ID NO: 46 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 43 or SEQ ID NO: 46, (ii) a variable heavy chain comprising the amino acid sequence of SEQ ID NO: 22 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 22, or (iii) a variable light chain as in (i) and a variable heavy chain as in (ii).

In some embodiments, the TNFα antibody comprises: (i) a variable light chain comprising the amino acid sequence of SEQ ID NO: 14, SEQ ID NO: 18, SEQ ID NO: 25, SEQ ID NO: 29, SEQ ID NO: 33, SEQ ID NO: 37, SEQ ID NO: 43, or SEQ ID NO: 46; (ii) a variable heavy chain comprising the amino acid sequence of SEQ ID NO: 22; or (iii) a variable light chain as in (i) and a variable heavy chain as in (ii).

In some embodiments, a TNFα antibody comprises a variable light chain comprising the amino acid sequence of SEQ ID NO: 14, SEQ ID NO: 18, SEQ ID NO: 25, SEQ ID NO: 29, SEQ ID NO: 33, SEQ ID NO: 37, SEQ ID NO: 43, or SEQ ID NO: 46.

In some embodiments, a TNFα antibody comprises a variable heavy chain comprising the amino acid sequence of SEQ ID NO: 22.

In some embodiments, the TNFα antibody comprises a variable light chain comprising the amino acid sequence of SEQ ID NO: 43 and a variable heavy chain comprising the amino acid sequence of SEQ ID NO: 22.

In some embodiments, the TNFα antibody comprises a variable light chain comprising the amino acid sequence of SEQ ID NO: 46 and a variable heavy chain comprising the amino acid sequence of SEQ ID NO: 22.

In some embodiments, the TNFα antibody comprises (i) a variable light chain comprising the amino acid sequence of SEQ ID NO: 55 or SEQ ID NO: 56 or a variant thereof wherein 1, 2, 3, 4, 5, or 6 amino acids of the variable light chain is substituted by a different amino acid, (ii) a variable heavy chain comprising the amino acid sequence of SEQ ID NO: 57 or SEQ ID NO: 58 or a variant thereof wherein 1, 2, 3, 4, 5, or 6 amino acids of the variable heavy chain is substituted by a different amino acid, or (iii) a variable light chain as in (i) and a variable heavy chain as in (ii).

In some embodiments, the TNFα antibody comprises (i) a variable light chain comprising the amino acid sequence of SEQ ID NO: 55 or SEQ ID NO: 56 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 55 or SEQ ID NO: 56, (ii) a variable heavy chain comprising the amino acid sequence of SEQ ID NO: 57 or SEQ ID NO: 58 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 57 or SEQ ID NO: 58, or (iii) a variable light chain as in (i) and a variable heavy chain as in (ii).

In some embodiments, the TNFα antibody comprises: (i) a variable light chain comprising the amino acid sequence of SEQ ID NO: 55 or SEQ ID NO: 56; (ii) a variable heavy chain comprising the amino acid sequence of SEQ ID NO: 57 or SEQ ID NO: 58; or (iii) a variable light chain as in (i) and a variable heavy chain as in (ii).

In some embodiments, a TNFα antibody comprises a variable light chain comprising the amino acid sequence of SEQ ID NO: 55 or SEQ ID NO: 56.

In some embodiments, a TNFα antibody comprises a variable heavy chain comprising the amino acid sequence of SEQ ID NO: 57 or SEQ ID NO: 58.

In some embodiments, a caninized NGF antibody comprises (i) a variable light chain comprising the amino acid sequence of SEQ ID NO: 73, a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 73; (ii) a variable heavy chain comprising the amino acid sequence of SEQ ID NO: 74 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 74; or (iii) a variable heavy chain as in (i) and a variable heavy chain as in (ii).

In some embodiments, a caninized NGF antibody comprises (i) a variable light chain comprising the amino acid sequence of SEQ ID NO: 73, a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 73; (ii) a variable heavy chain comprising the amino acid sequence of SEQ ID NO: 74 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 74; or (iii) a variable heavy chain as in (i) and a variable heavy chain as in (ii).

In some embodiments, a caninized NGF antibody comprises (i) a variable light chain sequence of 73 or a variant thereof wherein 1, 2, 3, 4, 5, or 6 amino acids of the variable light chain is substituted by a different amino acid, (ii) a variable heavy chain sequence of SEQ ID NO: 74 or a variant thereof wherein 1, 2, 3, 4, 5, or 6 amino acids of the variable heavy chain is substituted by a different amino acid, or (iii) a variable light chain sequence as in (i) and a variable heavy chain sequence as in (ii).

The term “constant region” as used herein refers to a region comprising at least three constant domains.

The terms “heavy chain constant region” or “constant heavy chain” are used interchangeably to refer to a region comprising at least three heavy chain constant domains, CH1, CH2, and CH3. Nonlimiting exemplary heavy chain constant regions include γ, δ, α, ε, and μ. Each heavy chain constant region corresponds to an antibody isotype. For example, an antibody comprising a γ constant region is an IgG antibody, an antibody comprising a δ constant region is an IgD antibody, an antibody comprising an α constant region is an IgA antibody, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to IgM1 and IgM2.

The terms “light chain constant region” or “constant light chain” are used interchangeably to refer to a region comprising a light chain constant domain, CL. Nonlimiting exemplary light chain constant regions include λ and κ. Non-function-altering deletions and alterations within the domains are encompassed within the scope of the term “constant region” unless designated otherwise. Canine, feline, and equine have antibody classes such as IgG, IgA, IgD, IgE, and IgM. Within the canine IgG antibody class are IgG-A, IgG-B, IgG-C, and IgG-D.

The term “chimeric antibody” or “chimeric” refers to an antibody in which a portion of the heavy chain or light chain is derived from a particular source or species, while at least a part of the remainder of the heavy chain or light chain is derived from a different source or species. In some embodiments, a chimeric antibody refers to an antibody comprising at least one variable region from a first species (such as mouse, rat, cynomolgus monkey, etc.) and at least one constant region from a second species (such as human, dog, cat, equine, etc.). In some embodiments, a chimeric antibody comprises at least one mouse variable region and at least one canine constant region. In some embodiments, all of the variable regions of a chimeric antibody are from a first species and all of the constant regions of the chimeric antibody are from a second species. In some embodiments, a chimeric antibody comprises a constant heavy chain region or constant light chain region from a companion animal. In some embodiments, a chimeric antibody comprises a mouse variable heavy and light chains and a companion animal constant heavy and light chains. For example, a chimeric antibody may comprise a mouse variable heavy and light chains and a canine constant heavy and light chains.

In some embodiments, a TNFα antibody comprises a chimeric antibody comprising: (a) (i) a light chain amino acid sequence of SEQ ID NO: 10; (ii) a heavy chain amino acid sequence of SEQ ID NO: 12; or (iii) a light chain amino acid sequence as in (i) and a heavy chain sequence as in (ii); or (b) (i) a light chain amino acid sequence of SEQ ID NO: 40; (ii) a heavy chain amino acid sequence of SEQ ID NO: 12; or (iii) a light chain amino acid sequence as in (i) and a heavy chain sequence as in (ii).

A “canine chimeric,” “chimeric canine” or “canine chimeric antibody” refers to a chimeric antibody having at least a portion of a heavy chain or a portion of a light chain derived from a dog. In some embodiments, a canine chimeric antibody comprises a mouse variable heavy and light chains and a canine constant heavy and light chains. In some embodiments, the antibody is a chimeric antibody comprising murine variable heavy chain framework regions or murine variable light chain framework regions.

In some embodiments, a TNFα and/or NGF antibody comprises a canine heavy chain constant region selected from an IgG-A, IgG-B, IgG-C, and IgG-D constant region.

A “caninized antibody” means an antibody in which at least one amino acid in a portion of a non-canine variable region has been replaced with the corresponding amino acid from a canine variable region. In some embodiments, a caninized antibody comprises at least one canine constant region (e.g., a γ constant region, an α constant region, a δ constant region, an c constant region, a μ constant region, or etc.) or fragment thereof. In some embodiments, a caninized antibody is an antibody fragment, such as Fab, scFv, (Fab′)₂, etc. The term “caninized” also denotes forms of non-canine (for example, murine) antibodies that are chimeric immunoglobulins, immunoglobulin chains, or fragments thereof (such as Fv, Fab, Fab′, F(ab′)₂ or other antigen-binding sequences of antibodies) that contain minimal sequence of non-canine immunoglobulin Caninized antibodies can include canine immunoglobulins (recipient antibody) in which residues from a CDR of the recipient are substituted by residues from a CDR of a non-canine species (donor antibody) such as mouse, rat, or rabbit having the desired specificity, affinity, and capacity. In some instances, Fv framework region (FR) residues of the canine immunoglobulin are replaced by corresponding non-canine residues. Furthermore, the caninized antibody can comprise residues that are found neither in the recipient antibody nor in the imported CDR or framework sequences, but are included to further refine and optimize antibody performance.

In some embodiments, at least one amino acid residue in a portion of a mouse variable heavy chain or a mouse variable light chain has been replaced with the corresponding amino acid from a canine variable region. In some embodiments, the modified chain is fused to a canine constant heavy chain or a canine constant light chain.

In some embodiments, a TNFα antibody is a caninized antibody comprising (i) a light chain sequence of SEQ ID NO: 15, SEQ ID NO: 19, SEQ ID NO: 26, SEQ ID NO: 30, SEQ ID NO: 34, SEQ ID NO: 38, or SEQ ID NO: 44 or a variant thereof having at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 15, SEQ ID NO: 19, SEQ ID NO: 26, SEQ ID NO: 30, SEQ ID NO: 34, SEQ ID NO: 38, or SEQ ID NO: 44, (ii) a heavy chain sequence of SEQ ID NO: 23 or SEQ ID NO: 64 or a variant thereof having at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 23 or SEQ ID NO: 64, or (iii) a light chain sequence as in (i) and a heavy chain sequence as in (ii).

In some embodiments, a TNFα antibody is a caninized antibody comprising (i) a light chain sequence of SEQ ID NO: 44, (ii) a heavy chain sequence of SEQ ID NO: 23 or SEQ ID NO: 64, or (iii) a light chain sequence as in (i) and a heavy chain sequence as in (ii).

In some embodiments, a TNFα antibody is a caninized antibody comprising (i) a light chain sequence of SEQ ID NO: 59 or SEQ ID NO: 60 or a variant thereof having at least at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 59 or SEQ ID NO: 60, (ii) a heavy chain sequence of SEQ ID NO: 61 or SEQ ID NO: 62 or a variant thereof having at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 61 or SEQ ID NO: 62, or (iii) a light chain sequence as in (i) and a heavy chain sequence as in (ii).

In some embodiments, a TNFα antibody is a caninized antibody comprising (i) a light chain sequence of SEQ ID NO: 59 or SEQ ID NO: 60, (ii) a heavy chain sequence of SEQ ID NO: 61 or SEQ ID NO: 62, or (iii) a light chain sequence as in (i) and a heavy chain sequence as in (ii).

In some embodiments, the caninized NGF antibody comprises: (i) a light chain sequence of SEQ ID NO: 77 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 77, (ii) a heavy chain sequence of SEQ ID NO: 79 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 79, or (iii) a light chain sequence as in (i) and a heavy chain sequence as in (ii).

In some embodiments, the caninized NGF antibody comprises a light chain comprising the amino acid sequence of SEQ ID NO: 77 and a heavy chain comprising the amino acid sequence of SEQ ID NO: 79.

In some embodiments, the antibody binds to canine TNFα and canine NGF and comprises (i) a variable light chain comprising the amino acid of SEQ ID NO: 14, SEQ ID NO: 18, SEQ ID NO: 25, SEQ ID NO: 29, SEQ ID NO: 33, SEQ ID NO: 37, SEQ ID NO: 43, SEQ ID NO: 46, SEQ ID NO: 55, or SEQ ID NO: 56 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: SEQ ID NO:

14, SEQ ID NO: 18, SEQ ID NO: 25, SEQ ID NO: 29, SEQ ID NO: 33, SEQ ID NO: 37, SEQ ID NO: 43, SEQ ID NO: 46, SEQ ID NO: 55, or SEQ ID NO: 56; (ii) a variable heavy chain comprising the amino acid sequence of SEQ ID NO: 22, SEQ ID NO: 57, or SED NO: 58 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 22, SEQ ID NO: 57, or SEQ ID NO: 58; (iii) a variable light chain comprising the amino acid sequence of SEQ ID NO: 73 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 73; (iv) a variable heavy chain comprising the amino acid sequence of SEQ ID NO: 74 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 74; or (v) a variable light chain sequence as in (i), a variable heavy chain sequence as in (ii), a variable light chain sequence as in (iii), and a variable heavy chain sequence as in (iv).

In some embodiments, the antibody binds to canine TNFα and canine NGF and comprises (i) a variable light chain comprising the amino acid of SEQ ID NO: 43 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 43; (ii) a variable heavy chain comprising the amino acid sequence of SEQ ID NO: 22 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 22; (iii) a variable light chain comprising the amino acid sequence of SEQ ID NO: 73 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 73; (iv) a variable heavy chain comprising the amino acid sequence of SEQ ID NO: 74 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 74; or (v) a variable light chain sequence as in (i), a variable heavy chain sequence as in (ii), a variable light chain sequence as in (iii), and a variable heavy chain sequence as in (iv).

In some embodiments, the antibody binds to canine TNFα and canine NGF and comprises (i) a variable light chain comprising the amino acid of SEQ ID NO: 43; (ii) a variable heavy chain comprising the amino acid sequence of SEQ ID NO: 22; (iii) a variable light chain comprising the amino acid sequence of SEQ ID NO: 73; and (iv) a variable heavy chain comprising the amino acid sequence of SEQ ID NO: 74.

In some embodiments, the antibody binds to canine TNFα and canine NGF and comprises the amino acid sequence of SEQ ID NO: 77 and the amino acid sequence of SEQ ID NO: 81.

A “fragment crystallizable polypeptide” or “Fc polypeptide” is the portion of an antibody molecule that interacts with effector molecules and cells. It comprises the C-terminal portions of the immunoglobulin heavy chains. As used herein, an Fc polypeptide includes fragments of the Fc domain having one or more biological activities of an entire Fc polypeptide. An “effector function” of the Fc polypeptide is an action or activity performed in whole or in part by any antibody in response to a stimulus and may include complement fixation and/or ADCC (antibody-dependent cellular cytotoxicity) induction and/or ADCP (antibody-dependent cellular phagocytosis).

In some embodiments, a biological activity of an Fc polypeptide is the ability to bind FcRn. In some embodiments, a biological activity of an Fc polypeptide is the ability to bind C1q. In some embodiments, a biological activity of an Fc polypeptide is the ability to bind CD16. In some embodiments, a biological activity of an Fc polypeptide is the ability to bind protein A.

The term “IgX Fc” means the Fc region is derived from a particular antibody isotype (e.g., IgG, IgA, IgD, IgE, IgM, etc.), where “X” denotes the antibody isotype. Thus, “IgG Fc” denotes the Fc region of a γ chain, “IgA Fc” denotes the Fc region of an α chain, “IgD Fc” denotes the Fc region of a δ chain, “IgE Fc” denotes the Fc region of an c chain, “IgM Fc” denotes the Fc region of a μ chain, etc. In some embodiments, the IgG Fc region comprises CH1, hinge, CH2, CH3, and CL1. “IgX-N-Fc” denotes that the Fc region is derived from a particular subclass of antibody isotype (such as canine IgG subclass A, B, C, or D; or feline IgG subclass 1, 2a, or 2b), where “N” denotes the subclass. In some embodiments, IgX Fc or IgX-N-Fc regions are derived from a companion animal, such as a dog. In some embodiments, IgG Fc regions are isolated from canine γ heavy chains, such as IgG-A, IgG-B, IgG-C, or IgG-D. Antibodies comprising an Fc region of IgG-A, IgG-B, IgG-C, or IgG-D may provide for higher expression levels in recombination production systems.

The terms “IgX Fc” and “IgX Fc polypeptide” include wild-type IgX Fc polypeptides and variant IgX Fc polypeptides, unless indicated otherwise.

In some embodiments, a variant IgG Fc polypeptide comprises a variant IgG Fc polypeptide of a companion animal species. In some embodiments, a variant IgG Fc polypeptide comprises a variant canine IgG Fc polypeptide. In some embodiments, a variant IgG Fc polypeptide (e.g., a variant canine IgG-A Fc polypeptide, a variant canine IgG-C Fc polypeptide, or a variant canine IgG-D Fc polypeptide, variant feline IgG1a Fc polypeptide) has an activity that the reference (e.g., wild-type) polypeptide substantially lacks.

An antibody may be modified to extend or shorten its half-life. In some embodiments involving a higher dose of antibody, a shorter half-life may be desirable for acute treatment. In some embodiments involving a lower dose of antibody, a longer half-life may be desirable for prolonged treatment. For example, as discussed below, mutations in IgG Fc that affect FcRn interactions may be introduced.

In some embodiments, a TNFα and/or NGF antibody comprises a wild-type or variant IgG Fc having complement fixation activity (or complement-dependent cytotoxicity (CDC)). In some embodiments, a TNFα and/or NGF antibody comprises a wild-type or variant IgG Fc having antibody-dependent cellular cytotoxicity (ADCC) activity. In some embodiments, a TNFα and/or NGF antibody comprises a wild-type or variant IgG Fc having antibody-dependent cellular phagocytosis (ADCP) activity. In some embodiments, a TNFα and/or NGF antibody comprises a wild-type or variant IgG Fc having complement fixation activity and/or ADCC activity and/or ADCP activity. IgG Fc polypeptides may be modified to have an effector function or to have an enhanced effector function.

In some embodiments, a TNFα and/or NGF antibody comprises a wild-type or variant IgG Fc the binds to canine FcRn at low pH.

In some embodiments, a variant IgG Fc (e.g., a variant canine IgG Fc polypeptide) has modified FcRn binding affinity compared to a reference polypeptide. In some embodiments, a variant IgG Fc has increased FcRn binding affinity at an acidic pH (e.g., at a pH in the range of from about 5.0 to about 6.5, such as at a pH of about 5.0, a pH of about 5.5, a pH of about 6.0, or a pH of about 6.5) compared to a reference polypeptide. Exemplary variant IgG Fc polypeptides having increased FcRn binding affinity are disclosed in WO 2020/082048, which is incorporated by reference herein in its entirety.

In some embodiments, a variant IgG Fc (e.g., a variant canine IgG Fc polypeptide) has modified Protein A binding affinity compared to a reference polypeptide. In some embodiments, a variant IgG Fc has increased Protein A binding affinity compared to a reference polypeptide. Exemplary variant IgG Fc polypeptides having increased Protein A binding affinity are disclosed in WO 2020/139984 (e.g., Example 2), which is incorporated by reference herein in its entirety.

The term “affinity” means the strength of the sum total of noncovalent interactions between a single binding site of a molecule (for example, an antibody) and its binding partner (for example, an antigen). The affinity of a molecule X for its partner Y can generally be represented by the dissociation constant (K D). Affinity can be measured by common methods known in the art, such as, for example, immunoblot, ELISA KD, KinEx A, biolayer interferometry (BLI), or surface plasmon resonance devices.

The terms “K_{D},” “K_{d},” “Kd” or “Kd value” as used interchangeably to refer to the equilibrium dissociation constant of an antibody-antigen interaction. In some embodiments, the K d of the antibody is measured by using biolayer interferometry assays using a biosensor, such as an Octet® System (Pall ForteBio LLC, Fremont, CA) according to the supplier's instructions.

For example, biotinylated antigen is bound to the sensor tip and the association of antibody is monitored for ninety seconds and the dissociation is monitored for 600 seconds. The buffer for dilutions and binding steps is 20 mM phosphate, 150 mM NaCl, pH 7.2. A buffer only blank curve is subtracted to correct for any drift. The data are fit to a 2:1 binding model using ForteBio data analysis software to determine association rate constant (kₒₙ), dissociation rate constant (k_{off}), and the K_{d}. The equilibrium dissociation constant (K_{d}) is calculated as the ratio of k_{off}/kₒₙ. The term “kₒₙ” refers to the rate constant for association of an antibody to an antigen and the term “k_{off}” refers to the rate constant for dissociation of an antibody from the antibody/antigen complex.

The term “binds” to an antigen or epitope is a term that is well understood in the art, and methods to determine such binding are also well known in the art. A molecule is said to exhibit “binding” if it reacts, associates with, or has affinity for a particular cell or substance and the reaction, association, or affinity is detectable by one or more methods known in the art, such as, for example, immunoblot, ELISA KD, KinEx A, biolayer interferometry (BLI), surface plasmon resonance devices, or etc.

“Surface plasmon resonance” denotes an optical phenomenon that allows for the analysis of real-time biospecific interactions by detection of alterations in protein concentrations within a biosensor matrix, for example using the BIAcore™ system (BIAcore International AB, a GE Healthcare company, Uppsala, Sweden and Piscataway, N.J.). For further descriptions, see Jonsson et al. (1993) Ann. Biol. Clin. 51: 19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is an Octet® system (Pall ForteBio LLC). See, e.g., Abdiche et al., 2008, Anal. Biochem. 377: 209-277.

In some embodiments, a TNFα antibody binds to canine TNFα, human TNFα, feline TNFα, or equine TNFα with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry.

In some embodiments, a TNFα antibody binds to canine TNFα, human TNFα, feline TNFα, or equine TNFα with a Kd of between 5×10⁻⁶ M and 1×10⁻⁶ M, between 5×10⁻⁶ M and 5×10⁻⁷ M, between 5×10⁻⁶ M and 1×10⁻⁷ M, between 5×10⁻⁶ M and 5×10⁻⁸ M, 5×10⁻⁶ M and 1×10⁻⁸ M, between 5×10⁻⁶ M and 5×10⁻⁹ M, between 5×10⁻⁶ M and 1×10⁻⁹ M, between 5×10⁻⁶ M and 5×10⁻¹⁰ M, between 5×10⁻⁶ M and 1×10⁻¹⁰ M, between 5×10⁻⁶ M and 5×10⁻¹¹ M, between 5×10⁻⁶ M and 1×10⁻¹¹ M, between 5×10⁻⁶ M and 5×10⁻¹² M, between 5×10⁻⁶ M and 1×10⁻¹² M, between 1×10⁻⁶ M and 5×10⁻⁷ M, between 1×10⁻⁶ M and 1×10⁻⁷ M, between 1×10⁻⁶ M and 5×10⁻⁸ M, 1×10⁻⁶ M and 1×10⁻⁸ M, between 1×10⁻⁶ M and 5×10⁻⁹ M, between 1×10⁻⁶ M and 1×10⁻⁹ M, between 1×10⁻⁶ M and 5×10⁻¹⁰ M, between 1×10⁻⁶ M and 1×10⁻¹⁰ M, between 1×10⁻⁶ M and 5×10⁻¹¹ M, between 1×10⁻⁶ M and 1×10⁻¹¹ M, between 1×10⁻⁶ M and 5×10⁻¹² M, between 1×10⁻⁶ M and 1×10⁻¹² M, between 5×10⁻⁷ M and 1×10⁻⁷ M, between 5×10⁻⁷ M and 5×10⁻⁸ M, 5×10⁻⁷ M and 1×10⁻⁸ M, between 5×10⁻⁷ M and 5×10⁻⁹ M, between 5×10⁻⁷ M and 1×10⁻⁹ M, between 5×10⁻⁷ M and 5×10⁻¹⁰ M, between 5×10⁻⁷ M and 1×10⁻¹⁰ M, between 5×10⁻⁷ M and 5×10⁻¹¹ M, between 5×10⁻⁷ M and 1×10⁻¹¹ M, between 5×10⁻⁷ M and 5×10⁻¹² M, between 5×10⁻⁷ M and 1×10⁻¹² M, between 1×10⁻⁷ M and 5×10⁻⁸ M, 1×10⁻⁷ M and 1×10⁻⁸ M, between 1×10⁻⁷ M and 5×10⁻⁹ M, between 1×10⁻⁷ M and 1×10⁻⁹ M, between 1×10⁻⁷ M and 5×10⁻¹⁰ M, between 1×10⁻⁷ M and 1×10⁻¹⁰ M, between 1×10⁻⁷ M and 5×10⁻¹¹ M, between 1×10⁻⁷ M and 1×10⁻¹¹ M, between 1×10⁻⁷ M and 5×10⁻¹² M, between 1×10⁻⁷ M and 1×10⁻¹² M, between 5×10⁻⁸ M and 1×10⁻⁸ M, between 5×10⁻⁸ M and 5×10⁻⁹ M, between 5×10⁻⁸ M and 1×10⁻⁹ M, between 5×10⁻⁸ M and 5×10⁻¹⁰ M, between 5×10⁻⁸ M and 1×10⁻¹⁰ M, between 5×10⁻⁸ M and 5×10⁻¹¹ M, between 5×10⁻⁸ M and 1×10⁻¹¹ M, between 5×10⁻⁸ M and 5×10⁻¹² M, between 5×10⁻⁸ M and 1×10⁻¹² M, 1×10⁻⁸ M and 5×10⁻⁹ M, between 1×10⁻⁸ M and 1×10⁻⁹ M, between 1×10⁻⁸ M and 5×10⁻¹⁰ M, between 1×10⁻⁸ M and 1×10⁻¹⁰ M, between 1×10⁻⁸ M and 5×10⁻¹¹ M, between 1×10⁻⁸ M and 1×10⁻¹¹ M, between 1×10⁻⁸ M and 5×10⁻¹² M, between 1×10⁻⁸ M and 1×10⁻¹² M, between 5×10⁻⁹ M and 1×10⁻⁹ M, between 5×10⁻⁹ M and 5×10⁻¹⁰ M, between 5×10⁻⁹ M and 1×10⁻¹⁰ M, between 5×10⁻⁹ M and 5×10⁻¹¹ M, between 5×10⁻⁹ M and 1×10⁻¹¹ M, between 5×10⁻⁹ M and 5×10⁻¹² M, between 5×10⁻⁹ M and 1×10⁻¹² M, between 1×10⁻⁹ M and 5×10⁻¹⁰ M, between 1×10⁻⁹ M and 1×10⁻¹⁰ M, between 1×10⁻⁹ M and 5×10⁻¹¹ M, between 1×10⁻⁹ M and 1×10⁻¹¹ M, between 1×10⁻⁹ M and 5×10⁻¹² M, between 1×10⁻⁹ M and 1×10⁻¹² M, between 5×10⁻¹⁰ M and 1×10⁻¹⁰ M, between 5×10⁻¹⁰ M and 5×10⁻¹¹ M, between, 1×10⁻¹⁰ M and 5×10⁻¹¹ M, 1×10⁻¹⁰ M and 1×10-11 M, between 1×10⁻¹⁰ M and 5×10⁻¹² M, between 1×10⁻¹⁰ M and 1×10⁻¹² M, between 5×10-11 M and 1×10⁻¹² M, between 5×10⁻¹¹ M and 5×10⁻¹² M, between 5×10⁻¹¹ M and 1×10⁻¹² M, between 1×10⁻¹¹ M and 5×10⁻¹² M, or between 1×10⁻¹¹ M and 1×10⁻¹² M, as measured by biolayer interferometry.

In some embodiments, a TNFα antibody binds to canine TNFα, human TNFα, feline TNFα, or equine TNFα, as determined by immunoblot analysis.

In some embodiments, a NGF antibody binds to canine NGF, human NGF, feline NGF, or equine NGF with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry.

In some embodiments, a NGF antibody binds to canine NGF, human NGF, feline NGF, or equine NGF with a Kd of between 5×10⁻⁶ M and 1×10⁻⁶ M, between 5×10⁻⁶ M and 5×10⁻⁷ M, between 5×10⁻⁶ M and 1×10⁻⁷ M, between 5×10⁻⁶ M and 5×10⁻⁸ M, 5×10⁻⁶ M and 1×10⁻⁸ M, between 5×10⁻⁶ M and 5×10⁻⁹ M, between 5×10⁻⁶ M and 1×10⁻⁹ M, between 5×10⁻⁶ M and 5×10⁻¹⁰ M, between 5×10⁻⁶ M and 1×10⁻¹⁰ M, between 5×10⁻⁶ M and 5×10⁻¹¹ M, between 5×10⁻⁶ M and 1×10⁻¹¹ M, between 5×10⁻⁶ M and 5×10⁻¹² M, between 5×10⁻⁶ M and 1×10⁻¹² M, between 1×10⁻⁶ M and 5×10⁻⁷ M, between 1×10⁻⁶ M and 1×10⁻⁷ M, between 1×10⁻⁶ M and 5×10⁻⁸ M, 1×10⁻⁶ M and 1×10⁻⁸ M, between 1×10⁻⁶ M and 5×10⁻⁹ M, between 1×10⁻⁶ M and 1×10⁻⁹ M, between 1×10⁻⁶ M and 5×10⁻¹⁰ M, between 1×10⁻⁶ M and 1×10⁻¹⁰ M, between 1×10⁻⁶ M and 5×10⁻¹¹ M, between 1×10⁻⁶ M and 1×10⁻¹¹ M, between 1×10⁻⁶ M and 5×10⁻¹² M, between 1×10⁻⁶ M and 1×10⁻¹² M, between 5×10⁻⁷ M and 1×10⁻⁷ M, between 5×10⁻⁷ M and 5×10⁻⁸ M, 5×10⁻⁷ M and 1×10⁻⁸ M, between 5×10⁻⁷ M and 5×10⁻⁹ M, between 5×10⁻⁷ M and 1×10⁻⁹ M, between 5×10⁻⁷ M and 5×10⁻¹⁰ M, between 5×10⁻⁷ M and 1×10⁻¹⁰ M, between 5×10⁻⁷ M and 5×10⁻¹¹ M, between 5×10⁻⁷ M and 1×10⁻¹¹ M, between 5×10⁻⁷ M and 5×10⁻¹² M, between 5×10⁻⁷ M and 1×10⁻¹² M, between 1×10⁻⁷ M and 5×10⁻⁸ M, 1×10⁻⁷ M and 1×10⁻⁸ M, between 1×10⁻⁷ M and 5×10⁻⁹ M, between 1×10⁻⁷ M and 1×10⁻⁹ M, between 1×10⁻⁷ M and 5×10⁻¹⁰ M, between 1×10⁻⁷ M and 1×10⁻¹⁰ M, between 1×10⁻⁷ M and 5×10⁻¹¹ M, between 1×10⁻⁷ M and 1×10⁻¹¹ M, between 1×10⁻⁷ M and 5×10⁻¹² M, between 1×10⁻⁷ M and 1×10⁻¹² M, between 5×10⁻⁸ M and 1×10⁻⁸ M, between 5×10⁻⁸ M and 5×10⁻⁹ M, between 5×10⁻⁸ M and 1×10⁻⁹ M, between 5×10⁻⁸ M and 5×10⁻¹⁰ M, between 5×10⁻⁸ M and 1×10⁻¹⁰ M, between 5×10⁻⁸ M and 5×10⁻¹¹ M, between 5×10⁻⁸ M and 1×10⁻¹¹ M, between 5×10⁻⁸ M and 5×10⁻¹² M, between 5×10⁻⁸ M and 1×10⁻¹² M, 1×10⁻⁸ M and 5×10⁻⁹ M, between 1×10⁻⁸ M and 1×10⁻⁹ M, between 1×10-8 M and 5×10⁻¹⁰ M, between 1×10⁻⁸ M and 1×10⁻¹⁰ M, between 1×10⁻⁸ M and 5×10⁻¹¹ M, between 1×10⁻⁸ M and 1×10⁻¹¹ M, between 1×10⁻⁸ M and 5×10⁻¹² M, between 1×10⁻⁸ M and 1×10⁻¹² M, between 5×10⁻⁹ M and 1×10⁻⁹ M, between 5×10⁻⁹ M and 5×10⁻¹⁰ M, between 5×10⁻⁹ M and 1×10⁻¹⁰ M, between 5×10⁻⁹ M and 5×10⁻¹¹ M, between 5×10⁻⁹ M and 1×10⁻¹¹ M, between 5×10⁻⁹ M and 5×10⁻¹² M, between 5×10⁻⁹ M and 1×10⁻¹² M, between 1×10⁻⁹ M and 5×10⁻¹⁰ M, between 1×10⁻⁹ M and 1×10⁻¹⁰ M, between 1×10⁻⁹ M and 5×10⁻¹¹ M, between 1×10⁻⁹ M and 1×10⁻¹¹ M, between 1×10⁻⁹ M and 5×10⁻¹² M, between 1×10⁻⁹ M and 1×10⁻¹² M, between 5×10⁻¹⁰ M and 1×10⁻¹⁰ M, between 5×10⁻¹⁰ M and 5×10⁻¹¹ M, between, 1×10⁻¹⁰ M and 5×10⁻¹¹ M, 1×10⁻¹⁰ M and 1×10⁻¹¹ M, between 1×10⁻¹⁰ M and 5×10⁻¹² M, between 1×10⁻¹⁰ M and 1×10⁻¹² M, between 5×10⁻¹¹ M and 1×10⁻¹² M, between 5×10⁻¹¹ M and 5×10⁻¹² M, between 5×10⁻¹¹ M and 1×10⁻¹² M, between 1×10⁻¹¹ M and 5×10⁻¹² M, or between 1×10⁻¹¹ M and 1×10⁻¹² M, as measured by biolayer interferometry.

In some embodiments, a NGF antibody binds to canine NGF, human NGF, feline NGF, or equine NGF, as determined by immunoblot analysis.

“Wild-type” refers to a non-mutated version of a polypeptide that occurs in nature, or a fragment thereof. A wild-type polypeptide may be produced recombinantly.

A “variant” means a biologically active polypeptide having at least about 50% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, deleted, at the N- or C-terminus of the polypeptide.

In some embodiments, a variant has at least 1, 2, 3, 4, 5, or 6 amino acids substituted by a different amino acid.

In some embodiments, a variant has at least about 50% sequence identity with the reference nucleic acid molecule or polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, deleted, at the N- or C-terminus of the polypeptide. In some embodiments, a variant has at least about 50% sequence identity, at least about 60% sequence identity, at least about 65% sequence identity, at least about 70% sequence identity, at least about 75% sequence identity, at least about 80% sequence identity, at least about 85% sequence identity, at least about 90% sequence identity, at least about 95% sequence identity, at least about 97% sequence identity, at least about 98% sequence identity, or at least about 99% sequence identity with the sequence of the reference nucleic acid or polypeptide.

As used herein, “percent (%) amino acid sequence identity” and “homology” with respect to a peptide, polypeptide, or antibody sequence are defined as the percentage of amino acid residues in a candidate sequence that are identical with the amino acid residues in the specific peptide or polypeptide sequence, after aligning the sequences and introducing gaps, if necessary to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALINE™ (DNASTAR) software. Those skilled in the art can determine appropriate parameters for measuring alignment, including any algorithms needed to achieve maximal alignment over the full length of sequences being compared.

An “amino acid substitution” refers to the replacement of one amino acid in a polypeptide with another amino acid. In some embodiments, an amino acid substitution is a conservative substitution. Nonlimiting exemplary conservative amino acid substitutions are shown in Table 2 Amino acid substitutions may be introduced into a molecule of interest and the products screened for a desired activity, for example, retained/improved antigen binding, decreased immunogenicity, or improved ADCC or CDC or enhanced pharmacokinetics.

TABLE 2

| Original Residue | Exemplary Substitutions |
|---|---|
| Ala (A) | Val; Leu; Ile |
| Arg (R) | Lys; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes with another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters or enhancers) that regulate the expression of the polypeptide of interest, or one or more selectable marker genes (such as, for example, antibiotic resistance genes and genes that can be used in colorimetric assays, for example, (3-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), 293 cells, and CHO cells, and their derivatives, such as 293-6E, DG44, CHO-S, and CHO-K cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) encoding an amino acid sequence(s) provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated.” In some embodiments, the TNFα and/or NGF antibody is purified using chromatography, such as size exclusion chromatography, ion exchange chromatography, protein A column chromatography, hydrophobic interaction chromatography, and CHT chromatography.

The term “companion animal species” refers to an animal suitable to be a companion to humans. In some embodiments, a companion animal species is a small mammal, such as a canine, feline, dog, cat, horse, rabbit, ferret, guinea pig, rodent, etc. In some embodiments, a companion animal species is a farm animal, such as a horse, cow, pig, etc.

To “reduce” or “inhibit” means to decrease, reduce, or arrest an activity, function, or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time. A “reference” as used herein, refers to any sample, standard, or level that is used for comparison purposes. A reference may be obtained from a healthy or non-diseased sample. In some examples, a reference is obtained from a non-diseased or non-treated sample of a companion animal. In some examples, a reference is obtained from one or more healthy animals of a particular species, which are not the animal being tested or treated.

The term “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 15% 20%, 25%, 30%, 35%, 40%, 45%, 50%, 60%, 70%, 80%, 90%, or 100% compared to the reference value.

In some embodiments, a TNFα antibody may reduce TNFα signaling function in a companion animal species by at least 10%, at least 15%, at least 20%, at least 25%, at least 30%, at least 35%, at least 40%, at least 45%, at least 50%, at least 60%, at least 70%, at least 80%, at least 90%, or 100% compared to TNFα signaling function in the absence of the antibody. In some embodiments, the reduction in TNFα signaling function is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

In some embodiments, a NGF antibody may reduce NGF signaling function in a companion animal species by at least 10%, at least 15%, at least 20%, at least 25%, at least 30%, at least 35%, at least 40%, at least 45%, at least 50%, at least 60%, at least 70%, at least 80%, at least 90%, or 100% compared to NGF signaling function in the absence of the antibody. In some embodiments, the reduction in NGF signaling function is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

Exemplary Pharmaceutical Compositions

The terms “pharmaceutical formulation” and “pharmaceutical composition” refer to a preparation which is in such form as to permit the biological activity of the active ingredient(s) to be effective, and which contains no additional components that are unacceptably toxic to a subject to which the formulation would be administered.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine.

The pharmaceutical composition can be stored in lyophilized form. Thus, in some embodiments, the preparation process includes a lyophilization step. The lyophilized composition may then be reformulated, typically as an aqueous composition suitable for parenteral administration, prior to administration to the dog. In other embodiments, particularly where the antibody is highly stable to thermal and oxidative denaturation, the pharmaceutical composition can be stored as a liquid, i.e., as an aqueous composition, which may be administered directly, or with appropriate dilution, to the dog. A lyophilized composition can be reconstituted with sterile Water for Injection (WFI). Bacteriostatic reagents, such benzyl alcohol, may be included. Thus, the invention provides pharmaceutical compositions in solid or liquid form.

The pH of the pharmaceutical compositions may be in the range of from about pH 5 to about pH 8, when administered. The compositions of the invention are sterile if they are to be used for therapeutic purposes. Sterility can be achieved by any of several means known in the art, including by filtration through sterile filtration membranes (e.g., 0.2 micron membranes). Sterility may be maintained with or without anti-bacterial agents.

Exemplary Uses of Antibodies and Pharmaceutical Compositions

The antibodies or pharmaceutical compositions comprising the antibodies of the invention may be useful for treating condition associated with TNFα and/or NGF.

As used herein, a “condition associated with TNFα” means a disease associated with, caused by, or characterized by, elevated levels or altered gradients of TNFα concentration. Such conditions include, but are not limited to, autoimmune disorders such ankylosing spondylitis, asthma, cancer, Crohn's disease, inflammatory bowel disease (IBD), juvenile idiopathic arthritis, psoriasis, including plaque psoriasis, psoriatic arthritis, rheumatoid arthritis, ulcerative colitis, and other chronic inflammatory disorders.

As used herein, a “condition associated with NGF” means a disease associated with, caused by, or characterized by, elevated levels or altered gradients of NGF concentration. Such conditions include, but are not limited to, pain such as chronic pain, acute pain, and/or inflammatory pain. In some embodiments, the pain is osteoarthrititic pain, back pain, cancer pain, and/or a neuropathic pain. In some embodiments, the pain is associated with a surgery, a broken or fractured bone, dental work, a burn, a cut, and/or labor.

A condition associated with TNFα and/or NGF may be exhibited in a companion animal, including, but not limited to, a canine.

As used herein, “treatment” is an approach for obtaining beneficial or desired clinical results. “Treatment” as used herein, covers any administration or application of a therapeutic for disease in a mammal, including a companion animal. For purposes of this disclosure, beneficial or desired clinical results include, but are not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In some embodiments, a TNFα and/or NGF antibody or pharmaceutical compositions comprising the same can be utilized in accordance with the methods herein to treat conditions associated with TNFα and/or NGF. In some embodiments, a TNFα and/or NGF antibody or pharmaceutical composition is administered to a companion animal, such as a canine, to treat a condition associated with TNFα and/or NGF. In some embodiments, a TNFα and/or NGF antibody or pharmaceutical composition is administered to a companion animal, such as a canine, to maintain remission of a condition associated with TNFα and/or NGF.

A “therapeutically effective amount” of a substance/molecule, agonist or antagonist may vary according to factors such as the type of disease to be treated, the disease state, the severity and course of the disease, the type of therapeutic purpose, any previous therapy, the clinical history, the response to prior treatment, the discretion of the attending veterinarian, age, sex, and weight of the animal, and the ability of the substance/molecule, agonist or antagonist to elicit a desired response in the animal A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic or prophylactic result.

In some embodiments, a TNFα and/or NGF antibody or pharmaceutical composition comprising a TNFα and/or NGF antibody is administered parenterally, by subcutaneous administration, intravenous infusion, or intramuscular injection. In some embodiments, a TNFα and/or NGF antibody or pharmaceutical composition comprising a TNFα and/or NGF antibody is administered as a bolus injection or by continuous infusion over a period of time. In some embodiments, a TNFα and/or NGF antibody or pharmaceutical composition comprising a TNFα and/or NGF antibody is administered by an intramuscular, an intraperitoneal, an intracerebrospinal, a subcutaneous, an intra-arterial, an intrasynovial, an intrathecal, or an inhalation route.

TNFα and/or NGF antibodies described herein may be administered in an amount in the range of 0.01 mg/kg body weight to 100 mg/kg body weight per dose. In some embodiments, TNFα and/or NGF antibodies may be administered in an amount in the range of 0.5 mg/kg body weight to 50 mg/kg body weight per dose. In some embodiments, TNFα and/or NGF antibodies may be administered in an amount in the range of 0.1 mg/kg body weight to 10 mg/kg body weight per dose. In some embodiments, TNFα and/or NGF antibodies may be administered in an amount in the range of 0.1 mg/kg body weight to 100 mg/kg body weight per dose. In some embodiments, TNFα and/or NGF antibodies may be administered in an amount in the range of 1 mg/kg body weight to 10 mg/kg body weight per dose. In some embodiments, TNFα and/or NGF antibodies may be administered in an amount in the range of 0.5 mg/kg body weight to 100 mg/kg body, in the range of 1 mg/kg body weight to 100 mg/kg body weight, in the range of 5 mg/kg body weight to 100 mg/kg body weight, in the range of 10 mg/kg body weight to 100 mg/kg body weight, in the range of 20 mg/kg body weight to 100 mg/kg body weight, in the range of 50 mg/kg body weight to 100 mg/kg body weight, in the range of 1 mg/kg body weight to 10 mg/kg body weight, in the range of 5 mg/kg body weight to 10 mg/kg body weight, in the range of 0.5 mg/kg body weight to 10 mg/kg body weight, in the range of 0.01 mg/kg body weight to 0.5 mg/kg body weight, in the range of 0.01 mg/kg body weight to 0.1 mg/kg body weight, or in the range of 5 mg/kg body weight to 50 mg/kg body weight. In some embodiments, TNFα and/or NGF antibodies may be administered in an amount of 0.5 mg/kg body weight. In some embodiments, TNFα and/or NGF antibodies may be administered in an amount of 2 mg/kg body weight.

A TNFα and/or NGF antibody or a pharmaceutical composition comprising a TNFα and/or NGF antibody can be administered to a companion animal at one time or over a series of treatments. For example, a TNFα and/or NGF antibody or a pharmaceutical composition comprising a TNFα and/or NGF antibody may be administered at least once, more than once, at least twice, at least three times, at least four times, or at least five times.

In some embodiments, the dose is administered once per week for at least two or three consecutive weeks, and in some embodiments, this cycle of treatment is repeated two or more times, optionally interspersed with one or more weeks of no treatment. In other embodiments, the therapeutically effective dose is administered once per day for two to five consecutive days, and in some embodiments, this cycle of treatment is repeated two or more times, optionally interspersed with one or more days or weeks of no treatment.

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

In some embodiments, the method comprises administering in combination with a TNFα and/or NGF antibody or a pharmaceutical composition comprising a TNFα and/or NGF antibody, an IL17 antibody, an IL-5 antibody, an IL-31 antibody, an IL4 antibody, an IL13 antibody, an IL23 antibody, an IgE antibody, a CD11 a antibody, an IL6R antibody, an α4-Intergrin antibody, an IL12 antibody, an IL113 antibody, or an anti-BlyS antibody.

Provided herein are methods of exposing to a cell a TNFα and/or NGF antibody or a pharmaceutical composition comprising a TNFα and/or NGF antibody under conditions permissive for binding of the antibody to TNFα and/or NGF. In some embodiments, the cell is exposed to the antibody or pharmaceutical composition ex vivo. In some embodiments, the cell is exposed to the antibody or pharmaceutical composition in vivo. In some embodiments, a cell is exposed to the TNFα and/or NGF antibody or the pharmaceutical composition under conditions permissive for binding of the antibody to intracellular TNFα and/or NGF. In some embodiments, a cell is exposed to the TNFα and/or NGF antibody or the pharmaceutical composition under conditions permissive for binding of the antibody to extracellular TNFα and/or NGF.

In some embodiments, a cell may be exposed in vivo to the TNFα and/or NGF antibody or the pharmaceutical composition by any one or more of the administration methods described herein, including but not limited to, intraperitoneal, intramuscular, intravenous injection into the subject. In some embodiments, a cell may be exposed ex vivo to the TNFα and/or NGF antibody or the pharmaceutical composition by exposing the cell to a culture medium comprising the antibody or the pharmaceutical composition. In some embodiments, the permeability of the cell membrane may be affected by the use of any number of methods understood by those of skill in the art (such as electroporating the cells or exposing the cells to a solution containing calcium chloride) before exposing the cell to a culture medium comprising the antibody or the pharmaceutical composition.

In some embodiments, the binding results in a reduction of TNFα and/or NGF signaling function by the cell. In some embodiments, a TNFα and/or NGF antibody may reduce TNFα and/or NGF signaling function in a cell by at least 10%, at least 15%, at least 20%, at least 25%, at least 30%, at least 35%, at least 40%, at least 45%, at least 50%, at least 60%, at least 70%, at least 80%, at least 90%, or 100% compared to TNFα and/or NGF signaling function in the absence of the antibody. In some embodiments, the reduction in TNFα and/or NGF signaling function is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

Provided herein are methods of using the TNFα and/or NGF antibodies, polypeptides and polynucleotides for detection, diagnosis and monitoring of a condition associated with TNFα and/or NGF. Provided herein are methods of determining whether a companion animal will respond to TNFα and/or NGF antibody therapy. In some embodiments, the method comprises detecting whether the animal has cells that express TNFα and/or NGF using a TNFα and/or NGF antibody. In some embodiments, the method of detection comprises contacting the sample with an antibody, polypeptide, or polynucleotide and determining whether the level of binding differs from that of a reference or comparison sample (such as a control). In some embodiments, the method may be useful to determine whether the antibodies or polypeptides described herein are an appropriate treatment for the subject animal.

In some embodiments, the sample is a biological sample. The term “biological sample” means a quantity of a substance from a living thing or formerly living thing. In some embodiments, the biological sample is a cell or cell/tissue lysate. In some embodiments, the biological sample includes, but is not limited to, blood, (for example, whole blood), plasma, serum, urine, synovial fluid, and epithelial cells.

In some embodiments, the cells or cell/tissue lysate are contacted with a TNFα and/or NGF antibody and the binding between the antibody and the cell is determined. When the test cells show binding activity as compared to a reference cell of the same tissue type, it may indicate that the subject would benefit from treatment with a TNFα and/or NGF antibody. In some embodiments, the test cells are from tissue of a companion animal.

Various methods known in the art for detecting specific antibody-antigen binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Appropriate labels include, without limitation, radionuclides (for example ¹²⁵I, ¹³¹I, ³⁵S, ³H, or ³²P), enzymes (for example, alkaline phosphatase, horseradish peroxidase, luciferase, or p-glactosidase), fluorescent moieties or proteins (for example, fluorescein, rhodamine, phycoerythrin, GFP, or BFP), or luminescent moieties (for example, Qdot™ nanoparticles supplied by the Quantum Dot Corporation, Palo Alto, Calif.). General techniques to be used in performing the various immunoassays noted above are known to those of ordinary skill in the art.

For purposes of diagnosis, the polypeptide including antibodies can be labeled with a detectable moiety including but not limited to radioisotopes, fluorescent labels, and various enzyme-substrate labels know in the art. Methods of conjugating labels to an antibody are known in the art. In some embodiments, the TNFα and/or NGF antibodies need not be labeled, and the presence thereof can be detected using a second labeled antibody which binds to the first TNFα and/or NGF antibody. In some embodiments, the TNFα and/or NGF antibody can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987). The TNFα and/or NGF antibodies and polypeptides can also be used for in vivo diagnostic assays, such as in vivo imaging. Generally, the antibody or the polypeptide is labeled with a radionuclide (such as ¹¹¹In, ⁹⁹Tc, ¹⁴C, ¹³¹I, ¹²⁵I, ³H, or any other radionuclide label, including those outlined herein) so that the cells or tissue of interest can be localized using immunoscintiography. The antibody may also be used as staining reagent in pathology using techniques well known in the art.

In some embodiments, a first antibody is used for a diagnostic and a second antibody is used as a therapeutic. In some embodiments, the first and second antibodies are different. In some embodiments, the first and second antibodies can both bind to the antigen at the same time, by binding to separate epitopes.

The following examples illustrate particular aspects of the disclosure and are not intended in any way to limit the disclosure.

### EXAMPLES

### Example 1

Synthesis and Purification of Caninized TNFα D2E7 Antibodies from CHO Cells

DNA sequences encoding caninized TNFα D2E7 antibodies are synthesized chemically and inserted into an expression vector suitable for transfection into a CHO host cell and secretion of a light chain or heavy chain protein or both from the cell. The expression vector(s) is/are transfected into a CHO cell. The CHO cells are selected for high yield and stability of expression of the TNFα antibody or component thereof, optionally using a DHFR gene on the expression vector and methotrexate-mediated gene amplification, as is known in the art. The CHO cells are cultured until sufficient quantities of the TNFα antibody are produced. The TNFα antibody is purified by one or more of various steps including Protein A column chromatography, Protein G column chromatography, Protein L column chromatography, or other chromatographic methods such as ion exchange column chromatography, hydrophobic interaction column chromatography, mixed mode column chromatography such as CHT, and/or multimodal mode column chromatography such as CaptoMMC. Low pH or other viral inactivation and viral removal steps can be applied. The purified protein is admixed with excipients, and sterilized by filtration to prepare a pharmaceutical composition of the invention. The pharmaceutical composition is administered to a dog, cat, or horse with a condition associated with TNFα in a dose sufficient to bind to inhibit TNFα.

More specifically, the variable light chain of TNFα antibody D2E7 (SEQ ID NO: 7) was caninized to SEQ ID NO: 14 (KBA VL v1) and SEQ ID NO: 18 (KBA VL v2) and the variable heavy chain of TNFα antibody D2E7 (SEQ ID NO: 8) was caninized to SEQ ID NO: 22 (KBA VH). DNA was synthesized to provide the translated protein sequences shown as SEQ ID NO: 15 (KBA VL v1 and canine kappa light constant region), SEQ ID NO: 19 (KBA VL v2 and canine kappa light constant region), and SEQ ID NO: 23 (caninized variable heavy chain of D2E7 and canine IgG-B constant region) and a leader sequence and used to prepare expression vectors for each. The light chain expression vectors had a different selection marker than that for the heavy chain. A light chain vector and heavy chain vector were co-transfected in various ratios to facilitate identification of a clone expressing the corresponding TNFα antibody efficiently.

The vectors were then used to perform pilot-scale transfection (1 L working volume) in CHO-KS cells using the FreestyleMax™ transfection reagent (Life Technologies). When cell viabilities dropped below 80%, the supernatant was harvested by clarifying the conditioned media. Protein was purified with a single pass Protein A chromatography step. From a 1L pilot-scale transient production, 12.2 mg of purified KBA v1 was obtained, while an even lower titer of 6.0 mg of KBA v2 was obtained. Both KBA v1 and KBA v2 caninized forms expressed low titers compared to typical ˜200 to 500 mg/L titers of other antibodies obtained in the lab. The purified KBA v1 and KBA v2 were analyzed by SDS-PAGE and HPLC gel filtration and appeared suitable for use. KBA v1 was further chosen to generate a stable cell line and low productivity was observed.

### Example 2

Demonstration of TNFα Binding Activity

This example demonstrates that TNFα antibodies described herein, illustrated with the TNFα antibodies KBA v1 also referred to herein as KIND-509 (SEQ ID NO: 15 and SEQ ID NO: 23) and KBA v2 (SEQ ID NO: 19 and SEQ ID NO: 23) bind TNFα with kinetics requisite for therapeutic activity.

The binding analysis was performed as follows. Briefly, canine and human TNFα were biotinylated. The free unreacted biotin was removed from biotinylated TNFα by extensive dialysis. Biotinylated TNFα was captured on streptavidin sensor tips. The association of five different concentrations (150, 50, 17, 5.6, and 1.9 nM) of TNFα antibody and TNFα (human and canine, in different tests) was monitored for ninety seconds. Dissociation was monitored for 600 seconds. A buffer only blank curve was subtracted to correct for any drift. The data were fit to a 1:1 binding model using ForteBio™ data analysis software to determine the kon (association rate constant), koff (dissociation rate constant) and the KD (dissociation constant). The binding statistics fell within acceptable parameters (Chi-squared less than or equal to 3.0; R-squared greater than or equal to 0.9). The buffer for dilutions and all binding steps was: 200 mM phosphate, 150 mM NaCl, 0.02% Tween-20, 0.05% sodium azide, and 0.1 mg BSA, pH7.4.

Canine TNFα was obtained from Sino Biological, cat. #7003-DNAE, lot #LCO5JU2002; human TNFα from Sigma, cat. #T6674; EZ-Link NHS-LC-biotin from Thermo Scientific, cat. #21336, lot #PB194183; and StreptAvidin biosensors from ForteBio, cat. #18-509, lot #1403251.

The binding kinetics were as follows. For the ligand canine TNFα, the KD (M) for KBA v1 was 9.88×10⁻¹¹ and 1.54×10⁻¹⁰ for adalimumab; the kon (1/Ms) was 8.39×10⁵ for KBA v1 and 7.22×10⁵ for adalimumab; the koff (1/s) was 8.28×10⁻⁵ for KBA v1 and 1.12×10⁻⁴ for adalimumab; the Rmax (nm) was 0.77 for KBA v1 and 0.76 for adalimumab; the Full Chi-squared was 0.14 for both KBA v1 and adalimumab; and the Full R-squared was 1 for both KBA v1 and adalimumab. For KBA v2, these values were: KD 3.03×10⁻¹⁰; kon 7.13×10⁵; koff 2.16×10⁴; Rmax 0.71; Full Chi-squared 0.38; and Full R-squared 1. Thus, KBA v1 has greater affinity to canine TNFα than that of KBA v2.

For the ligand human TNFα, the KD (M) for KBA v1 was 2.07×10⁻¹¹ and 3.06×10⁻¹¹ for adalimumab; the kon (1/Ms) was 7.41×10⁵ for KBA v1 and 6.74×10⁵ for adalimumab; the k_{off} (1/s) was 1.53×10⁻⁵ for KBA v1 and 2.06×10⁻⁵ for adalimumab; the Rmax (nm) was 1.05 for KBA v1 and 1.06 for adalimumab; the Full Chi-squared was 0.30 for KBA v1 and 0.27 for adalimumab; and the Full R-squared was 1 for both KBA v1 and adalimumab. For KBA v2, these values were: KD 2.69×10⁻¹⁰; kon 5.75×10⁵; koff 1.50×10⁻⁴; Rmax 0.98; Full Chi-squared 0.23; and Full R-squared 1.

TABLE 3
KBA v1 and KBA v2 interaction with canine
TNFα: binding kinetic data summary

| | | KD | kon | koff | Rmax | Full | Full |
|---|---|---|---|---|---|---|---|
| Antibody | Ligand | (M) | (1/Ms) | (1/s) | (nm) | Chi² | R² |
| KBA v1 | cTNFα | 9.88E−11 | 8.39E+05 | 8.28E−05 | 0.77 | 0.14 | 1 |
| KBA v2 | cTNFα | 3.03E−10 | 7.13E+05 | 2.16E−04 | 0.71 | 0.38 | 1 |

### Example 3

Identification of Caninization Issues Affecting Antibody Production

Chimeric D2E7 antibody comprising a chimeric variable light chain of D2E7 and canine kappa (SEQ ID NO: 10) and a chimeric variable heavy chain of D2E7 and canine IgG-B (SEQ ID NO: 12) was prepared according to the methods of Example 1. A typical expression level was observed from a 1L pilot-scale transient production. Hence, canine kappa and canine IgG-B did not appear to affect production.

To identify whether the caninized variable light chain or caninized variable heavy chain affected antibody production, antibodies comprising a caninized light chain paired with a chimeric heavy chain, and a chimeric light chain paired with a caninized heavy chain were prepared according to the methods of Example 1. Specifically, caninized variable heavy chain and canine IgG-B (SEQ ID NO: 23) was paired with chimeric variable light chain of D2E7 and canine kappa (SEQ ID NO: 10). The 1L pilot-scale production titer was typical, indicating that caninization of the variable heavy chain did not affect antibody production. However, when KBA VL v1 and canine kappa (SEQ ID NO: 15) was paired with chimeric variable heavy chain of D2E7 and canine IgG-B (SEQ ID NO: 12) the resulting 1L pilot-scale antibody production titer was low. In addition, KBA VL v2 and canine kappa (SEQ ID NO: 19) paired with chimeric variable heavy chain of D2E7 and canine IgG-B (SEQ ID NO: 12) resulted in an undetectable or very low 1L pilot-scale production titer. The low titer of these latter two antibodies suggests that caninization of the variable light chain led to the significant reduction observed in antibody production. Despite low production titers, the antibodies maintained TNFα binding activity.

### Example 4

Caninization of Variable Light Chain to Restore Antibody Production and Maintain TNFα Binding Activity

The amino acid(s) and position(s) of the variable light chain affecting antibody production were investigated by preparing a series of antibodies having different caninized variable light chains. Specifically, the following additional caninized variable light chains of D2E7 were designed: KBA VL v3 (SEQ ID NO: 25); KBA VL v4 (SEQ ID NO: 29); KBA VL v5 (SEQ ID NO: 33); KBA VL v6 (SEQ ID NO: 37); and KBA VL v7 (SEQ ID NO: 43). Each of KBA VL v3-v7 and canine kappa (SEQ ID NOs: 26, 30, 34, 38, and 44 respectively) was paired with chimeric variable heavy chain of D2E7 and canine IgG-B (SEQ ID NO: 12) or caninized variable heavy chain and canine IgG-B (SEQ ID NO: 23) and the resulting 1L pilot-scale transient antibody production in CHO cells was accessed. The antibody comprising KBA VL v7 (SEQ ID NO: 43) exhibited restored productivity and maintained binding activity to canine TNFα and human TNFα.

Further, when chimeric variable light chain of D2E7 and canine lambda (SEQ ID NO: 41) was paired with chimeric variable heavy chain of D2E7 and canine IgG-B (SEQ ID NO: 12) or caninized variable heavy chain and canine IgG-B (SEQ ID NO: 23), the resulting 1L pilot-scale antibody production titer showed no improvement.

A stable CHO cell line expressing TNFα antibody comprising KBA VL v7 and canine kappa (SEQ ID NO: 44) and caninized variable heavy chain and canine IgG-B (SEQ ID NO: 23) was generated. The antibody production titer in a shaker flask was greater than 4 g/L.

Framework region 2 of the light chain appears to affect antibody production. In particular, two residues—glutamine (Q) at position 3 of light chain framework region 2 (LC-PR2) and lysine (K) at position 8 of LC-FR2—are important for expression of caninized D2E7 light chain in anti-TNFα antibody production.

### Example 5

Additional Exemplary Caninized TNFα antibody

Additional caninized variable light chains of D2E7 may be designed having a glutamine (Q) at position 3 and a lysine (K) at position 8 of LC-FR2 (e.g., SEQ ID NO: 45). For example, another exemplary caninized variable light chain of D2E7 may be represented by SEQ ID NO: 46 (KBA VL v8).

### Example 6

Study for Treatment of Canine IBD with TNFα Antibodies

A multi-site, randomized, blinded, placebo-controlled, pilot, clinical field study is conducted to evaluate the effectiveness and safety of KIND-509 for the management of inflammatory bowel disease (IBD) in client-owned dogs.

The study involves dogs of any age, breed, weight, or sex that are diagnosed with IBD and meet the inclusion and exclusion criteria set forth below. The initial dose of a caninized TNFαantibody (e.g., KIND-509 or any other caninized TNFα antibody described herein) administered is 2 mg/kg based on body weight (BW) determined at Visit 1 (Day 0) and rounded to the nearest tenths place. All subsequent doses of caninized TNFα antibody are 1 mg/kg based on BW determined at Visits 2, 3 and 4 and rounded to the nearest tenths place. The control product (CP, e.g., PBS or formulation buffer) dose volume matches that of the treatment group. All dogs receive four doses of either caninized TNFα antibody or CP, with the first dose administered at Visit 1 (Day 0). Subsequent doses are administered every 7 (±2) days at Visits 2, 3, and 4.

Histopathology of endoscopic gastrointestinal biopsies are used to determine the effectiveness of caninized TNFα antibody in management of IBD.

Housing and General Management

Enrolled dogs reside with their owners and spend at least 60% of time inside the home. Only one dog from a multidog household is enrolled in the study at a time. Boarding is to be avoided during the study, and if boarding is necessary, boarding at the study site is ideal.

Water: Water is provided at the discretion of the owner on a schedule and in amounts that are customary for their dog. Neither sampling nor analysis of water is required.

Diet: A food trial of at least 14 consecutive days duration with a specified diet is completed within 90 days prior to the Screening Visit to rule out food responsive enteropathy. During the food trial, the prescription diet is the only diet fed. The Investigator determines if the food trial is adequate to rule out food responsive enteropathy. Food trial details are recorded at the Screening Visit. The food trial is not required if the dog has a Canine Inflammatory Bowel Disease Activity Index (CIBDAI) score of ≥6 and the Investigator deems the dog to have unstable disease (e.g., fever, severe weight loss, anorexia, etc.).

Ideally, dogs receive the specified diet as the sole diet when the dog is screened for the study, however, this is not a requirement for study inclusion.

The diet brand and formula is recorded at the Screening Visit. There should be no significant changes to the diet brand or formula within 14 days prior to the Screening Visit or throughout the duration of the study. The Investigator determines if a significant diet change has occurred. At each subsequent visit, Study Site Personnel record if the diet has changed significantly. Whether to terminate a dog that has a significant diet change prior to Visit 5 from the study is determined on a case-by-case basis in consultation with the Sponsor.

Inclusion Criteria: The dog:
Is manageable and co-operative with study procedures
Meets the water specifications described above
Has a history of at least one sign of IBD (weight loss, inappetence/anorexia, vomiting, or diarrhea) for at least three weeks prior to the Screening Visit and has at least one sign of IBD (weight loss, inappetence/anorexia, vomiting, or diarrhea) concurrently at the Screening Visit
Has a CIBDAI score of ≥4 (mild to severe disease) at the Screening Visit
Has been diagnosed with IBD based on histopathologic evaluation of endoscopic gastrointestinal biopsies obtained at the Screening Visit
Has received treatment with fenbendazole (50 mg/kg) per os (PO) for three consecutive days, within 60 days prior to the Screening Visit
Has completed at least a two-week (14 consecutive days) food trial with a specified diet deemed adequate by the Investigator within 90 days prior to the Screening Visit. This inclusion criteria is required if the dog has a CIBDAI score of ≥6 and the Investigator deems the dog to have unstable disease (e.g., fever, severe weight loss, anorexia, etc.)
Has received treatment with an anti-diarrheal antibiotic such as metronidazole or tylosin for at least 14 consecutive days, within 90 days prior to the Screening Visit; dogs receiving anti-diarrheal antibiotics at the Screening Visit must have antibiotics discontinued and wait seven days prior to enrollment/randomization (Visit 1). This inclusion criteria is required if the dog has a CIBDAI score of ≥6 and the Investigator deems the dog to have unstable disease (e.g. fever, severe weight loss, anorexia, etc.)
Is expected to live at least six months in the opinion of the Investigator

Exclusion Criteria: The dog:
Has a history of significant hypoalbuminernia (albumin <2.0 mg/dL) or has significant hypoalburninemia at the Screening Visit (albumin<2.0 mg/dL)
Has a history of ultrasonographic diagnosis of pancreatitis within six months prior to the Screening Visit
Has a history of, suspicion of, or diagnosis of exocrine pancreatic insufficiency
Has a history of abdominal surgery within six months prior to the Screening Visit
Has clinically significant hepatic or renal disease, neoplasia, diabetes mellitus, or other concurrent disease that might prevent completion of the study/interfere with study results
Has clinical signs of, or has been diagnosed with an infection (other than gingivitis or periodontal disease) and/or treated with antibiotics for an infection (other than anti-diarrheal antibiotics) within 30 days prior to the Screening Visit
Has suspicion of or diagnosed with hyperadrenocorticism, hypoadrenocorticism, or hypothyroidism
Has had a significant diet change within 14 days prior to the Screening Visit (significance of diet change will be determined by the Investigator)
Has received treatment with any of the following products:
   Systemic non-steroidal anti-inflammatory drugs within 14 days prior to the Screening Visit
   Pancreatic enzyme supplementation within 30 days prior to the Screening Visit
   Systemic, topical, ocular, or inhaled glucocorticoids or mineralocorticoids within 30 days prior to the Screening Visit
   Long-acting glucocorticoids within 90 days prior to the Screening Visit
   Cyclosporine, azathioprine, mycophenolate mofetil, or other immunosuppressive agent within 90 days prior to the Screening Visit
   Chlorambucil within 90 days prior to the Screening Visit
Is a pregnant or lactating female or a dog intended for breeding
Has been enrolled in another clinical trial within 30 days prior to the Screening Visit

Removal Criteria: The Investigator completes a Study Termination eCRF for all dogs that were screened, and the date of termination is recorded. Study Termination for each enrolled dog who completes the study is defined as the conclusion of the scheduled assessments and procedures and the last date that data is collected from the dog or the Owner. Laboratory results are not received until after the termination date. For all randomized cases who complete the study, Visit 5 is the study termination visit. If a dog is randomized and is removed prior to Visit 5, all procedures expected to be performed at Visit 5 are performed whenever possible. The reason for removal is noted on the Study Termination eCRF.

Post-Inclusion Removal Criteria: An enrolled dog may be removed early from further participation in the study for the following reasons:
The diet changes significantly
The dog requires treatment with a prohibited medication
The dog experiences an adverse event that precludes it from continuing in the study
The dog meets the criteria for removal based on the rescue clause below
The dog becomes non-compliant with study procedures
The dog is found to have been enrolled in error
The Owner is non-compliant with study procedures
The dog misses one or more scheduled study visits or exceeds the required time interval between treatments or requires monitoring procedures such that the response to the treatment cannot be evaluated
The Owner electively withdraws the dog from the study
The Sponsor decides to stop the study
Other reasons such as loss of contact with the Owner, change of residence, etc.

When possible, the Investigator and the Sponsor consult prior to removing a dog from the study early.

Rescue Clause: If the CIBDAI score at Visit 3 (Day 14±2) is the same as or higher than the CIBDAI score at the Screening Visit and the overall condition of IBD has worsened in the opinion of the Investigator, the dog is removed from the study and treated with appropriate standard of care therapy as directed by the Investigator. These dogs are considered treatment failures.

Animal Disposition and Withdrawal: Dogs terminated from the study remain in the custody of their Owner. The remains of dogs following euthanasia or spontaneous death (and post-mortem examination, if applicable) are returned to the Owner as desired or otherwise disposed of according to the study site policies as authorized by the Owner.

Study Schedule: The Screening Phase of the study is up to 10 days and the Treatment Phase of the study is 28±2 days resulting in a total study duration of 38±2 days. The Schedule of Events are outlined in Table 4, below. Unscheduled Visits are allowed at any time while the dog is enrolled in the study for further evaluation or as needed due to adverse events.

TABLE 4
Study Schedule of Events

| | | Visit 1 | | | | Visit 5 |
|---|---|---|---|---|---|---|
| | Screening | Enrollment/ | | | | Effectiveness |
| | Visit* | Randomization | Visit 2 | Visit 3 | Visit 4 | Endpoint |
| Event | (Day −10 to −1) | (Day 0) | (Day 7 ± 2) | (Day 14 ± 2) | (Day 21 ± 2) | (Day 28 ± 2) |
| Obtain Owner Consent | ✓ | | | | | |
| Review Medical History, Signalment | ✓ | | | | | |
| PE** | ✓ | ✓ | ✓ | ✓ | ✓ | ✓ |
| CIBDAI score | ✓ | ✓ | ✓ | ✓ | ✓ | ✓ |
| Owner Assessment | ✓^{α} | ✓ | ✓ | ✓ | ✓ | ✓ |
| Hematology and Biochemistry | ✓^{β} | | | ✓^{γ} | | ✓^{γ} |
| Cortisol | ✓^{β} | | | | | |
| TLI, cobalamin, and folate | ✓^{β} | | | | | |
| UA | ✓ | | | | | ✓ |
| Urine Culture ± Sensitivity | ✓ | | | | | |
| Histoplasma antigen EIA (urine) | ✓ | | | | | |
| Abdominal ultrasound | ✓ | | | | | |
| Gastroduodenoscopy and Colonoscopy*** | ✓ | | | | | |
| Inclusion/Exclusion Criteria | | ✓ | | | | |
| Enrollment/Randomization | | ✓ | | | | |
| IVP/CP Dose | | ✓ | ✓ | ✓ | ✓ | |
| Cyanocobalamin Dose | ✓ | ✓^{†} | ✓^{†} | ✓^{†} | ✓^{†} | |
| Record Concomitant Meds | ✓ | ✓ | ✓ | ✓ | ✓ | ✓ |
| Record Adverse Events | | ✓ | ✓ | ✓ | ✓ | ✓ |
| Review of Owner Diary | | ✓ | ✓ | ✓ | ✓ | ✓ |
| Study Termination | | | | | | ✓ |

*= The Screening Visit may take place over the course of two to four days (not required to be consecutive) to allow for completion and review of diagnostic testing required prior to pursuing preparation for colonoscopy, anesthesia and gastroduodenoscopy and colonoscopy and to allow for preparation for colonoscopy. While diagnostic testing is pending, Owners may take the dog home and return one to two days later to begin preparation for colonoscopy. All requirements of the Screening Visit must be completed within a time frame that will allow receipt and review of histopathology by Visit 1 (Day 0).
**= BW measured after fasting > 2 hours
***= The Screening Visit signalment, medical history, PE, CIBDAI score, Owner assessment and abdominal ultrasound must be completed prior to preparation for colonoscopy. In addition to the afore mentioned, the hematology, biochemistry, and cortisol must also be completed and reviewed prior to pursuing anesthesia and gastroduodenoscopy and colonoscopy.
^{α}= Baseline assessment completed at the Screening Visit
^{β}= measured after fasting > 8 hours
^{γ}= measured after fasting > 6 hours
^{†}= continued supplementation of cyanocobalamin is based on cobalamin concentration determined at the Screening Visit

Study Procedures/Assessments

Medical History: To distinguish adverse events from ongoing or pre-existing conditions, all significant medical conditions (including chronic or recurring conditions) which occurred within the 12 months prior to the Screening Visit, and any significant events which occurred before that, are recorded on the Medical History eCRF for each dog. This record includes a brief description of each condition, with the onset and resolution dates, if these dates are available. For conditions which are ongoing at the Screening Visit, the resolution date may be recorded during the study or listed as ongoing, as applicable.

As part of the medical history, the Investigator assesses the dog's history of clinical signs related to IBD including, but not limited to, lethargy, dehydration, inappetence/anorexia, weight loss, vomiting, diarrhea, tenesmus, hematochezia, and/or mucoid stools. The Investigator also classifies the dog's diarrhea as small bowel, large bowel, or mixed bowel diarrhea.

Physical Examination: The signalment of each dog screened for enrollment is recorded on the Signalment eCRF to include date of birth, breed, and gender (including whether intact or neutered).

Physical Examinations (PE) are performed at all scheduled study visits (Screening Visit and Visits 1-5) and any unscheduled visit, if necessary. Examinations include a subjective assessment of general appearance and attitude, hydration status, and the otic, ocular, oral, mucous membranes, respiratory, cardiovascular, gastrointestinal, neurological, musculoskeletal, lymphatic, integumentary, and genitourinary systems. Physical examination results are recorded on the Physical Examination eCRF. Abnormalities recorded after Visit 1 (Day 0) (excluding pre-existing conditions) and worsening abnormalities are reviewed and recorded as adverse events as described below.

Vital Signs: Temperature (° F.), heart rate (HR) (beats/minute), and respiratory rate (RR) (breaths/minute) are assessed with each PE and recorded on the Physical Examination eCRF.

Body Weight and Scales: Body weight is recorded in kilograms (kg) during each PE on the Physical Examination eCRF. For all dogs, body weight should be measured in a fasted state defined as BW obtained at least two hours after a meal.

Body Condition Score: Body Condition Score (BCS) is measured as part of the PE. The Nestle PURINA Body Condition System (LaFlamme, DP, 1997) is utilized to assess BCS. The BCS uses visualization and palpation to assess the general shape of the dog along with the amount of fat coverage over the ribs, spine, and hips. This BCS is on a scale of 1-9; a score of 9 is an extremely overweight dog and a score of 1 is an extremely underweight dog. Each dog is preferably assessed by the same Investigator at each visit.

Muscle Condition Score: Muscle Condition Score (MCS) is measured as part of the PE. The World Small Animal Veterinary Association (WSAVA) Muscle Condition Score system (WSAVA, 2014) is utilized to assess MCS. The MCS uses visualization and palpation of the spine, scapulae, skull, and wings of the ileum to grade the muscle mass as normal, mild loss, moderate loss, or severe loss. Each dog is preferably assessed by the same Investigator at each visit.

Canine Inflammatory Bowel Disease Activity Index (CIBDAI) Score: The Canine Inflammatory Bowel Disease Activity Index (CIBDAI) is a scoring system developed to evaluate the activity/severity of canine IBD. When utilizing the CIBDAI, six gastrointestinal signs are scored from 0-3 in dogs with IBD in comparison to normal. Scores from each of the six gastrointestinal tract signs are summed creating a cumulative CIBDAI score. Based on the CIBDAI score, IBD is classified as clinically insignificant, mild, moderate, or severe (Appendix 1).

At each visit, the Investigator completes a CIBDAI Score eCRF. The Investigator completes the CIBDAI score considering the Owner's report/assessment of clinical signs, the PE, changes in BW, etc. The Investigator assesses attitude/activity, appetite, vomiting, stool consistency, stool frequency, and weight loss compared to normal for the dog at the Screening Visit. At all subsequent visits, the Investigator rescores attitude/activity, appetite, vomiting, stool consistency, stool frequency, and weight loss compared to baseline. Preferably, the same Owner is interviewed, and the same Investigator completes the CIBDAI Score eCRF at each visit.

Owner Assessment: At the Screening Visit, the Owner completes an Owner Assessment—Baseline CRF of their dog's clinical signs (activity, appetite, vomiting, stool consistency, stool frequency) and overall quality of life (QoL) that most closely represents the dog's clinical signs and QoL over the two weeks preceding the Screening Visit. At each subsequent scheduled visit, the Owner completes an Owner Assessment CRF to note changes in their dog's clinical signs since the Screening Visit. The Owner Assessment—Baseline CRF and Owner Assessment CRF are transcribed to the Owner Assessment—Baseline eCRF and Owner Assessment eCRF by Study Site Personnel. Preferably, the same Owner completes the assessment at each visit. If the dog is boarded during the scheduled visit time, the Owner assessment is not completed, and an explanation is recorded in a note to file (NTF).

Stool consistency is graded based on the Purina Fecal Scoring system (VET 1502B-06FSC/15-6843, Société des Produits Nestlé S.A., Vevey, Switzerland). At the Screening Visit, the Owner is shown the Purina Fecal Scoring chart and reports the fecal score that most closely represents the dog's stool consistency over the two weeks preceding the Screening Visit. At each subsequent visit, the owner is shown the Purina Fecal Scoring chart and reports the fecal score that most closely represents the dog's stool consistency since the previous visit.

Clinical Pathology

Central Laboratory Clinical Pathology: Blood is collected via venipuncture at multiple study visits as specified in Table 3, and any unscheduled visits if necessary. Blood and urine samples are processed and sent as per the Central Laboratory guidance. At the Screening Visit, blood samples are collected after a fast of >8 hours. At Visits 3 and 5, blood samples are collected after a fast of >6 hours. All blood samples are collected prior to administration of caninized TNFα antibody or CP and cyanocobalamin.

The following tests are performed at the Central Laboratory:

Hematology (Completed at the Screening Visit and Visits 3 and 5): Total leukocyte count (WBC), Differential leukocyte count, Erythrocyte count (RBC), Hematocrit (Hct), Mean corpuscular volume (MCV), Mean corpuscular hemoglobin (MCH), Mean corpuscular hemoglobin concentration (MCHC), Hemoglobin (Hgb), and Platelet count.

Biochemistry (Completed at the Screening Visit and Visits 3 and 5): Amylase, Alanine aminotransferase (ALT), Lipase, Alkaline phosphatase (ALP), Cholesterol, Serum aspartate aminotransferase (AST), Creatinine, Total protein, Glucose, Albumin, Calcium, Globulin, Sodium, Total bilirubin, Chloride, Potassium, Blood urea nitrogen (BUN), Phosphorus, Creatine phosphokinase (CPK), Magnesium, Gamma-glutamyl transferase (GGT), and Triglyceride.

Cortisol (Completed at the Screening Visit): Blood is collected via venipuncture at the Screening Visit for assessment of cortisol concentration. The blood sample is processed and sent as per the Central Laboratory guidance.

Trypsin-like Immunoreactivity (TLI), Cobalamin, and Folate (Completed at the Screening Visit): Blood is collected via venipuncture at the Screening Visit for assessment of TLI, cobalamin and folate concentrations. The blood samples are processed and sent as per the Central Laboratory guidance. Dogs receive a cyanocobalamin injection at the Screening Visit after blood is drawn. Dogs with low cobalamin concentration based on Screening Visit results are treated with cyanocobalamin supplementation as described further below.

Urinalysis (UA) and Urine Culture ±Sensitivity (Completed at the Screening Visit and Visit 5 (for urinalysis) and the Screening Visit (for urine culture ±sensitivity): A complete UA (including urine specific gravity, dip strip and urine sediment evaluation) and urine culture ±sensitivity is evaluated. Urine is ideally collected by cystocentesis; however, if cystocentesis is not possible, urine can be collected by catheterization or voiding.

Additional laboratory assessments may be completed at the Investigator's discretion.

Histoplasma Antigen Enzyme Immunoassay: A minimum of 1 mL of urine is collected for histoplasma antigen enzyme immunoassay (EIA) at the Screening Visit.

Abdominal Ultrasound: An abdominal ultrasound is performed at the Screening Visit prior to preparing for colonoscopy, anesthesia, and gastroduodenoscopy and colonoscopy.

Gastroduodenoscopy and Colonoscopy and Endoscopic Gastrointestinal Biopsies: Gastroduodenoscopy and colonoscopy are performed at the Screening Visit. A minimum of six endoscopic biopsies are collected from the stomach, duodenum, and colon. If possible, the ileum is evaluated endoscopically, and biopsies are collected with endoscopic guidance or blindly. The Screening Visit may take place over the course of two to four days (not required to be consecutive) to allow for completion and review of diagnostic testing required prior to pursuing preparation for colonoscopy, anesthesia and gastroduodenoscopy and colonoscopy and to allow for preparation for colonoscopy. While diagnostic testing is pending, Owners may take the dog home and return one to two days later to begin preparation for colonoscopy.

The Screening Visit signalment, medical history, PE, CIBDAI score, Owner assessment and abdominal ultrasound are completed prior to preparation for colonoscopy. In addition to the afore mentioned, the hematology, biochemistry, and cortisol are also completed and reviewed prior to pursuing anesthesia and gastroduodenoscopy and colonoscopy.

All requirements of the Screening Visit are completed within a time frame that allows for receipt and review of histopathology by Visit 1 (Day 0).

Histopathology of Endoscopic Gastrointestinal Biopsies: Endoscopic gastrointestinal biopsies are submitted for histopathologic evaluation to the pathologist of the Investigator's choice. Histopathology diagnostic for idiopathic IBD (e.g., lymphoplasmacytic enteritis, eosinophilic enteritis, etc.) is required for the dog to be eligible for enrollment into the study.

Concomitant Medications and Therapies: Medications knowingly prescribed or administered one month prior to the Screening Visit and through Termination are considered a concomitant medication. Medications administered prior to study start are reviewed to ensure the dog meets study inclusion.

The Clinical Development Manager or assigned Study Monitor is contacted prior to administration of any concomitant medication for which the acceptable use is unclear.

Allowed Medications: New medications not expected to interfere with the treatment are allowed during the study. This includes, but may not be limited to, the following medications:
Fluid therapy (e.g., Lactated Ringers, 0.9% NaCl, etc.)
Antibiotics not intended to treat diarrhea
Antihistamines (e.g., diphenhydramine)
Preventive treatments (e.g., flea control, heart worm preventative, etc.)

Conditionally Allowed Medications: Existing medications are allowed if there were no prescription changes for two weeks prior to the Screening Visit and during the study. This includes and is limited to, the following medications:
Antiemetics (e.g., metoclopramide, ondansetron, maropitant, mirtazapine, etc.)
Antacids (e.g., omeprazole, famotidine, ranitidine, cimetidine, etc.)
Prokinetic agents (e.g., cisapride, erythromycin, metoclopramide, ranitidine, etc.)
Probiotics (e.g., Forti Flora, Proviable, etc.)
Opioids prescribed for analgesia (e.g., tramadol, buprenorphine, etc.)

An exception to the requirement for existing medications is made for antiemetics administered during the Screening Visit for endoscopy. Antiemetics administration are allowed at the time of endoscopy during the Screening Visit.

Cyanocobalamin Supplementation: All dogs receive an injection of cyanocobalamin 25 μg/kg subcutaneous (SQ) at the Screening Visit after blood samples are taken. Dogs determined to be deficient in cobalamin based on the cobalamin concentration at the Screening Visit receive cyanocobalamin 25 μg/kg SQ at Visits 1, 2, 3, and 4. The cyanocobalamin dose at any visit is not to exceed a total dose of 1500 μg. Cyanocobalamin is provided to the study sites by the Sponsor.

Prohibited Medications: Medications expected to interfere with the treatment and/or clinical signs of IBD are not allowed during the study. This includes, but may not be limited to, the following medications:
Vaccinations Glucocorticoids (e.g., prednisone, prednisolone, dexamethasone, triamcinolone,
betamethasone, etc.)
Mineralocorticoids (e.g., desoxycorticosterone pivalate, fludrocortisone, etc.)
Immunosuppressive agents (e.g., cyclosporine, azathioprine, mycophenolate mofetil, etc.)
Alkylating agents (e.g., chlorambucil, etc.)
Systemic non-steroidal anti-inflammatory drugs (e.g., carprofen, deracoxib, etc.)
Anti-diarrheal antibiotics (e.g., metronidazole, tylosin, sulfasalazine, etc.)
Anti-diarrheal medications (e.g., loperamide, Lomotil, etc.)
Pancreatic enzyme supplements

Owner Diary: Owners record any unfavorable or unexpected events observed throughout the study from the Screening Visit to Visit 5/Study Termination on the Owner Diary: Observed Events CRF. The Owner Diary: Observed Events CRF is reviewed by the Investigator at each visit. Observations listed by the Owner prior to Visit 1 are not recorded as adverse events. For each observation listed by the Owner after Visit 1, the Investigator indicates if the observation constitutes an adverse event as defined below.

Adverse Events: An adverse event (or AE) is any observation in a dog, whether or not considered to be IVP related, that is unfavorable and unintended and that occurs after any use of a veterinary medicinal product. Events fitting this description are considered AEs if they occur on or between the first treatment administration at Visit 1 (Day 0) and Study Termination.

Any new clinically significant (CS) laboratory result(s) (or the corresponding disease/condition) determined from samples collected after the dog received study treatment are recorded as an AE on the Adverse Events eCRF for each case. Not clinically significant (NCS) laboratory result(s) are not recorded as AEs.

Using the Adverse Events eCRF, the Investigator documents and describes AEs, to include the clinical sign/abnormality, start/end date or ongoing, study drug action taken, assess the relationship of the AE to the study drug, seriousness of the event, reason considered a SAE (if applicable), and outcome of event. The Sponsor codes each AE using Veterinary Dictionary for Drug Regulatory Authorities (VeDDRA) terminology. The Sponsor evaluates and performs final classification of AEs as serious or non-serious on a case by case basis at the end of the study.

The Investigator contacts the Monitor for a SAE and/or in the case of any unusually high frequency non-serious AEs observed.

Serious Adverse Events (SAE): A SAE is any AE that is fatal, or life-threatening, or requires professional intervention and is considered by the Investigator to be clinically serious, or causes abortion, stillbirth, infertility, congenital anomaly, or prolonged or permanent disability or disfigurement. The Owner contacts the Investigator as soon as reasonably possible following occurrence of a SAE. The Investigator assesses the case and determines the need for treatment. A description of the SAE is noted on the Adverse Events eCRF.

Causality Assessment: The Investigator completes a causality assessment for each AE using the following criteria:

Category A—Probable: All of the following apply:
There is reasonable association in time between the administration of the IVP and onset and duration of the reported event;
The description of the clinical phenomenon should be consistent with, or at least plausible, given the known pharmacology and toxicology of the product;
There should be no other equally plausible explanation(s) of the case. In particular, concurrent use of other products (and possible interactions) or intercurrent disease should be taken into account in the assessment.

Category B—Possible: The causality is one (of other) possible and plausible causes for the described AE, but the data does not meet the criteria for inclusion in Category A.

Category O—Unclassifiable/unassessable: Reliable data concerning an AE is unavailable or is insufficient to make an assessment of causality.

Category O1—Inconclusive: An association with the IVP cannot be discounted, but other factors prevent a conclusion from being drawn.

Category N—Unlikely: Sufficient information exists to establish beyond reasonable doubt that there is an alternative explanation to the AE that is not related to the IVP.

Necropsy and Histopathology

If necessary, to determine cause of death, or cause of the morbidity which led to euthanasia, and only after obtaining voluntary written consent from the Owner, a necropsy may be performed on any enrolled dog that died prior to study completion. Necropsy may include a complete gross examination for pathologic changes and tissue collection for histopathology.

Tissues collected at necropsy for histopathology may include: heart chambers, lungs, liver, gall bladder, stomach, duodenum, jejunum, ileum, colon/rectum, spleen, right and left kidneys, adrenals, bladder, and any other tissues deemed necessary.

Analysis Populations

Effectiveness Population: Effectiveness population consists of all enrolled dogs who were eligible for effectiveness analysis. Criteria for eligibility include completing the study through and including Visit 5. Criteria for ineligibility include discontinuation, for reason other than safety and effectiveness, and protocol violations affecting effectiveness assessment. All effectiveness analyses are based on the effectiveness population. The decision whether to include or exclude the effectiveness data for each dog terminated from the study prior to the primary effectiveness endpoint is determined on a case-by-case basis.

Safety Population: Safety population consists of all enrolled dogs who received at least one dose of caninized TNFα antibody or CP. Concomitant medication, AEs and IVP exposure are summarized based on the safety population.

Effectiveness Outcomes

Primary Variable: Reduction in CIBDAI score is the primary effectiveness variable. Disease remission and response are defined as follows:
Complete remission: Reduction in CIBDAI score of ≥75% or CIBDAI score <4
Partial remission: Reduction in CIBDAI score of ≥25% to <75%
Non-response: Reduction in CIBDAI score of <25%, no change in CIBDAI score, or an increase in CIBDAI score

Response or remission is assessed at Visit 5 compared to the CIBDAI score obtained at the Screening Visit. Additional exploratory statistical analyses on components of the CIBDAI score are conducted at the discretion of the Sponsor.

The effect of caninized TNFα antibody on the percent of dogs with a disease response and the percent of dogs in remission are evaluated using Fisher's Exact test (the FREQ procedure in SAS, SAS Institute, Cary NC; version 9.4 or higher). In addition, CIBDAI scores collected across time are subject to repeated measures analysis of covariance (RMANCOVA), with treatment group, time and the treatment by time interactions as fixed effect, and the baseline value included as a covariate. A compound symmetric structure is assumed for the covariance matrix. Given the small sample size, site is ignored in this analysis. If the interaction is significant, within time treatment effects are assessed. If the interaction was not significant, the main effect of treatment is evaluated.

Secondary Variables: Additional variables such as improvement/increase in BW, improvement/increase in BCS, improvement/increase in MCS, increase in appetite, decrease in vomiting, decreased frequency of bowel movements, improved fecal score, etc. are assessed. These outcomes are summarized as appropriate and may be subject to statistical analysis at the discretion of the Sponsor.

Safety Outcomes

Adverse Event and Concomitant Medications: Adverse event and concomitant medication data is summarized and tabulated for final reporting.

Clinical Pathology Laboratory Data: Each clinical pathology laboratory variable is summarized as appropriate. Urinalysis outcomes are summarized.

Vital Signs, Body Weight Data and Categorical Observations: Vital signs and BW data are evaluated as described above for clinical pathology outcomes. Outcomes categorical in nature, including AEs, are summarized by frequency and counts.

### Example 7

Preliminary Analysis of Treatment of Canine IBD with TNFα Antibodies

Ten dogs diagnosed with IBD completed the study described in Example 6 through Visit 5. The primary effectiveness variable was reduction in Canine Inflammatory Bowel Disease Activity Index (CIBDAI) score, which was assessed at Screening and Days 0, 7, 14, 21 and 28. Complete remission, defined as >75% reduction in average post-dose CIBDAI score from baseline, was achieved in 75% of the TNFα antibody group (KIND-509 at a dose of 2 mg/kg) compared to 17% in the placebo group. The treatment effect was early-onset and durable. At Day 7, the first post-dose visit, 75% of the KIND-509 treated dogs showed >75% reduction of CIBDAI score from baseline, compared to 17% in the placebo group. Furthermore, 50% of the KIND-509 treated dogs achieved and maintained 100% reduction of CIBDAI score from baseline throughout all post-dose visits, whereas none in the placebo group achieved the same result.

### Example 8

Design of Caninized NGF αD11 Antibodies

The variable light chain of αD11 rat anti-NGF monoclonal antibody (SEQ ID NO: 71) was caninized to SEQ ID NO: 73 and the variable heavy chain of αD11 rat anti-NGF monoclonal antibody (SEQ ID NO: 72) was caninized to SEQ ID NO: 74. This resulted in two amino acid changes in the variable heavy chain CDR-H1 (see SEQ ID NO: 75). Exemplary light and heavy chains comprising these caninized variable light and heavy chain sequences were also designed (SEQ ID NOs: 76-79).

### Example 9

Expression and Purification of Bispecific Anti-canine TNF and Anti-canine NGF Molecule from CHO Cells

To target TNF and NGF at the same time to reduce inflammation and reduce pain associated with conditions such as osteoarthritis, chronic pain, lower back pain, cancer pain, and neuropathic pain, bispecific molecules were designed.

An exemplary bispecific molecule designed comprises (1) caninized anti-NGF variable light chain with canine kappa light constant region (SEQ ID NO: 77) and (2) SEQ ID NO: 81 comprising caninized anti-NGF variable heavy chain (SEQ ID NO: 74), a variant canine IgG-B Fc engineered to be long-acting with reduced C1q and CD16 binding, and a single-chain variable fragment (ScFv) of caninized anti-TNF variable heavy (SEQ ID NO: 22) and light (SEQ ID NO: 43) chains.

The molecules (SEQ ID Nos 77 and 81) were expressed from mammalian cells and purified by single step Protein A column chromatography. Binding analysis was performed using a biosensor Octet. The Kd of the bispecific molecule comprising SEQ ID NO: 77 and SEQ ID NO: 81 was 8.38×10⁻¹⁰ for canine TNF-alpha was in the single digit pM range for canine NGF.

A second exemplary bispecific molecule was designed in the reverse comprising (1) caninized anti-TNF variable light chain with canine kappa light constant region (SEQ ID NO: 44) and (2) SEQ ID NO: 83 comprising caninized anti-TNF variable heavy chain (SEQ ID NO: 22), a variant canine IgG-B Fc engineered to be long-acting with reduced C1q and CD16 binding, and a single-chain variable fragment (ScFv) of caninized anti-NGF variable heavy (SEQ ID NO: 74) and light (SEQ ID NO: 73) chains.

Surprisingly, no expression was observed for the bispecific molecule in reverse (i.e., comprising SEQ ID NOs: 44 and 83).

1. An isolated antibody that binds to canine TNFα, wherein the antibody is a caninized antibody comprising a variable light chain comprising (i) a CDR-L1 comprising the amino acid sequence of SEQ ID NO: 1; (ii) a CDR-L2 comprising the amino acid sequence of SEQ ID NO: 2; (iii) a CDR L3 comprising the amino acid sequence of SEQ ID NO: 3; and (iv) a LC-FR2 comprising a glutamine at position 3 and a lysine at position 8.

2. The isolated antibody of claim 1, wherein the antibody comprises a variable heavy chain comprising (i) a CDR-H1 comprising the amino acid sequence of SEQ ID NO: 4; (ii) a CDR-H2 comprising the amino acid sequence of SEQ ID NO: 5; and (iii) a CDR H3 comprising the amino acid sequence of SEQ ID NO: 6.

3. The isolated antibody of claim 1, wherein the LC-FR2 comprises the amino acid sequence of SEQ ID NO: 45.

4. The isolated antibody of claim 1, wherein the antibody comprises (i) a variable light chain comprising the amino acid sequence of SEQ ID NO: 43 or SEQ ID NO: 46 or a variant thereof wherein 1, 2, 3, 4, 5, or 6 amino acids of the variable light chain is substituted by a different amino acid, (ii) a variable heavy chain comprising the amino acid sequence of SEQ ID NO: 22 or a variant thereof wherein 1, 2, 3, 4, 5, or 6 amino acids of the variable heavy chain is substituted by a different amino acid, or (iii) a variable light chain as in (i) and a variable heavy chain as in (ii).

5. The isolated antibody of claim 1, wherein the antibody comprises (i) a variable light chain comprising the amino acid sequence of SEQ ID NO: 43 or SEQ ID NO: 46 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 43 or SEQ ID NO: 46, (ii) a variable heavy chain comprising the amino acid sequence of SEQ ID NO: 22 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 22, or (iii) a variable light chain as in (i) and a variable heavy chain as in (ii).

6. An isolated antibody that binds to canine TNFα, wherein the antibody comprises: (i) a variable light chain comprising the amino acid sequence of SEQ ID NO: 14, SEQ ID NO: 18, SEQ ID NO: 25, SEQ ID NO: 29, SEQ ID NO: 33, SEQ ID NO: 37, SEQ ID NO: 43, or SEQ ID NO: 46; (ii) a variable heavy chain comprising the amino acid sequence of SEQ ID NO: 22; or (iii) a variable light chain as in (i) and a variable heavy chain as in (ii).

7. An isolated antibody that binds to canine TNFα, wherein the antibody comprises a variable light chain comprising the amino acid sequence of SEQ ID NO: 14, SEQ ID NO: 18, SEQ ID NO: 25, SEQ ID NO: 29, SEQ ID NO: 33, SEQ ID NO: 37, SEQ ID NO: 43, or SEQ ID NO: 46 and a variable heavy chain comprising the amino acid sequence of SEQ ID NO: 22.

8. An isolated antibody that binds to canine TNFα, wherein the antibody comprises a variable light chain comprising the amino acid sequence of SEQ ID NO: 43 and a variable heavy chain comprising the amino acid sequence of SEQ ID NO: 22.

9. An isolated antibody that binds to canine TNFα, wherein the antibody comprises a variable light chain comprising the amino acid sequence of SEQ ID NO: 46 and a variable heavy chain comprising the amino acid sequence of SEQ ID NO: 22.

10. An isolated antibody that binds to canine TNFα, wherein the antibody is a caninized antibody comprising:
(a) a light chain comprising (i) a CDR-L1 comprising the amino acid sequence of SEQ ID NO: 49; (ii) a CDR-L2 comprising the amino acid sequence of SEQ ID NO: 50; (iii) a CDR L3 comprising the amino acid sequence of SEQ ID NO: 51; and (iv) a variable light chain comprising the amino acid sequence of SEQ ID NO: 55 or SEQ ID NO: 56 or a variant thereof wherein 1, 2, 3, 4, 5, or 6 amino acids of the variable light chain is substituted by a different amino acid;
(b) a heavy chain comprising (i) a CDR-H1 comprising the amino acid sequence of SEQ ID NO: 52; (ii) a CDR-H2 comprising the amino acid sequence of SEQ ID NO: 53; (iii) a CDR H3 comprising the amino acid sequence of SEQ ID NO: 54; and (iv) a variable heavy chain comprising the amino acid sequence of SEQ ID NO: 57 or SEQ ID NO: 58 or a variant thereof wherein 1, 2, 3, 4, 5, or 6 amino acids of the variable heavy chain is substituted by a different amino acid; or
(c) a light chain as in (a) and a heavy chain as in (b).

11. (canceled)

12. (canceled)

13. (canceled)

14. (canceled)

15. The isolated antibody of claim 1, wherein the antibody comprises: (i) a light chain comprising the amino acid sequence of SEQ ID NO: 15, SEQ ID NO: 19, SEQ ID NO: 26, SEQ ID NO: 30, SEQ ID NO: 34, SEQ ID NO: 38, or SEQ ID NO: 44 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 15, SEQ ID NO: 19, SEQ ID NO: 26, SEQ ID NO: 30, SEQ ID NO: 34, SEQ ID NO: 38, or SEQ ID NO: 44, (ii) a heavy chain comprising the amino acid sequence of SEQ ID NO: 23 or SEQ ID NO: 64 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 23 or SEQ ID NO: 64, or (iii) a light chain as in (i) and a heavy chain as in (ii).

16. The isolated antibody of claim 1, wherein the antibody comprises: (i) a light chain comprising the amino acid sequence of SEQ ID NO: 44, (ii) a heavy chain comprising the amino acid sequence of SEQ ID NO: 23 or SEQ ID NO: 64, or (iii) a light chain as in (i) and a heavy chain as in (ii).

17. (canceled)

18. (canceled)

19. An isolated antibody that binds to canine TNFα, wherein the antibody is a caninized antibody comprising:
(a) a light chain comprising (i) a CDR-L1 comprising the amino acid sequence of SEQ ID NO: 49; (ii) a CDR-L2 comprising the amino acid sequence of SEQ ID NO: 50; (iii) a CDR L3 comprising the amino acid sequence of SEQ ID NO: 51; and (iv) a light chain comprising the amino acid sequence of SEQ ID NO: 59 or SEQ ID NO: 60 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 59 or SEQ ID NO: 60;
(b) a heavy chain comprising (i) a CDR-H1 comprising the amino acid sequence of SEQ ID NO: 52; (ii) a CDR-H2 comprising the amino acid sequence of SEQ ID NO: 53; (iii) a CDR H3 comprising the amino acid sequence of SEQ ID NO: 54; and (iv) a heavy chain comprising the amino acid sequence of SEQ ID NO: 61 or SEQ ID NO: 62 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 61 or SEQ ID NO: 62; or
(c) a light chain as in (a) and a heavy chain as in (b).

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. The antibody of claim 1, wherein the antibody is an antibody fragment selected from Fv, scFv, Fab, Fab′, F(ab′)2, and Fab′-SH.

32. An isolated antibody that binds to canine TNFα and canine NGF, wherein the antibody comprises (i) a variable light chain comprising the amino acid of SEQ ID NO: 14, SEQ ID NO: 18, SEQ ID NO: 25, SEQ ID NO: 29, SEQ ID NO: 33, SEQ ID NO: 37, SEQ ID NO: 43, SEQ ID NO: 46, SEQ ID NO: 55, or SEQ ID NO: 56 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: SEQ ID NO: 14, SEQ ID NO: 18, SEQ ID NO: 25, SEQ ID NO: 29, SEQ ID NO: 33, SEQ ID NO: 37, SEQ ID NO: 43, SEQ ID NO: 46, SEQ ID NO: 55, or SEQ ID NO: 56; (ii) a variable heavy chain comprising the amino acid sequence of SEQ ID NO: 22, SEQ ID NO: 57, or SED NO: 58 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 22, SEQ ID NO: 57, or SEQ ID NO: 58; (iii) a variable light chain comprising the amino acid sequence of SEQ ID NO: 73 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 73; (iv) a variable heavy chain comprising the amino acid sequence of SEQ ID NO: 74 or a variant thereof having at least 90%, at least 91%, at least 92%, at least 93%, at least 94%, at least 95%, at least 96%, at least 97%, at least 98%, or at least 99% sequence identity to the amino acid sequence of SEQ ID NO: 74; or (v) a variable light chain sequence as in (i), a variable heavy chain sequence as in (ii), a variable light chain sequence as in (iii), and a variable heavy chain sequence as in (iv).

33. (canceled)

34. (canceled)

35. An isolated nucleic acid or nucleic acids encoding the antibody of claim 1.

36. (canceled)

37. (canceled)

38. A pharmaceutical composition comprising the antibody of claim 1 and a pharmaceutically acceptable carrier.

39. A method of (1) treating a canine having a condition associated with TNFα, (2) a method of maintaining remission of a condition associated with TNFα in a canine, a (3) a method of treating a canine having a condition associated with NGF, (4) a method of maintaining remission of a condition associated with NGF in a canine, (5) each of the methods comprising administering to the canine a therapeutically effective amount of the antibody of claim 1.

40. (canceled)

41. (canceled)

42. (canceled)

43. The method of claim 40, wherein the condition associated with TNFα is a gastrointestinal inflammatory disease, an inflammatory disease, an inflammatory bowel disease, is ankylosing spondylitis, asthma, cancer, Crohn's disease, idiopathic arthritis, psoriasis, plaque psoriasis, psoriatic arthritis, rheumatoid arthritis, or ulcerative colitis.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. A method for detecting TNFα and/or NGF in a sample from a companion animal species comprising contacting the sample with the antibody of claim 1 under conditions permissive for binding of the antibody to TNFα and/or NGF, and detecting whether a complex is formed between the antibody and TNFα and/or NGF in the sample.

62. (canceled)


SEQUENCE LISTING (83 sequences)

SEQ ID NO: 1 - protein, 11 aa, Artificial Sequence
  Variable light chain CDR L1 of D2E7
RASQGIRNYLA

SEQ ID NO: 2 - protein, 6 aa, Artificial Sequence
  Variable light chain CDR L2 of D2E7
AASTLQ

SEQ ID NO: 3 - protein, 9 aa, Artificial Sequence
  Variable light chain CDR L3 of D2E7
QRYNRAPYT

SEQ ID NO: 4 - protein, 9 aa, Artificial Sequence
  Variable heavy chain CDR H1 of D2E7
FTFDDYAMH

SEQ ID NO: 5 - protein, 18 aa, Artificial Sequence
  Variable heavy chain CDR H2 of D2E7
AITWNSGHIDYADSVEGR

SEQ ID NO: 6 - protein, 14 aa, Artificial Sequence
  Variable heavy chain CDR H3 of D2E7
AKVSYLSTASSLDY

SEQ ID NO: 7 - protein, 107 aa, Artificial Sequence [sequence omitted]

SEQ ID NO: 8 - protein, 121 aa, Artificial Sequence [sequence omitted]

SEQ ID NO: 9 - protein, 237 aa, Artificial Sequence
  Chimeric variable light chain of D2E7 and canine kappa light constant region with leader
METDTLLLWVLLLWVPGSTGDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 10 - protein, 217 aa, Artificial Sequence
  Chimeric variable light chain of D2E7 and canine kappa light constant region
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 11 - protein, 475 aa, Artificial Sequence
  Chimeric variable heavy chain of D2E7 and canine IgG-B constant region with leader
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 12 - protein, 456 aa, Artificial Sequence
  Chimeric variable heavy chain of D2E7 and canine IgG-B constant region
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 13 - protein, 239 aa, Artificial Sequence
  Caninized variable light chain of D2E7 v1 and canine kappa light constant region with leader
MDMRVPAQLLGLLLLWLRGARCDIVMTQTPLSLSASPGEPVSISCRASQGIRNYLAWYLQKPGQSPRLLIYAASTLQSGVPDRFSGSGSGTDFTLRIGRLEAEDAAIYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 14 - protein, 107 aa, Artificial Sequence
  Caninized variable light chain of D2E7 v1 (KBA VL v1)
DIVMTQTPLSLSASPGEPVSISCRASQGIRNYLAWYLQKPGQSPRLLIYAASTLQSGVPDRFSGSGSGTDFTLRIGRLEAEDAAIYYCQRYNRAPYTFGQGTKVEIK

SEQ ID NO: 15 - protein, 217 aa, Artificial Sequence
  Caninized variable light chain of D2E7 v1 and canine kappa light constant region
DIVMTQTPLSLSASPGEPVSISCRASQGIRNYLAWYLQKPGQSPRLLIYAASTLQSGVPDRFSGSGSGTDFTLRIGRLEAEDAAIYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 16 - protein, 15 aa, Artificial Sequence
  Variable light chain framework region LC-FR2 of KBA VL v1
WYLQKPGQSPRLLIY

SEQ ID NO: 17 - protein, 239 aa, Artificial Sequence
  Caninized variable light chain of D2E7 v2 and canine kappa light constant region with leader
MDMRVPAQLLGLLLLWLRGARCDIVMTQTPPSLSASPGEPVSISCRASQGIRNYLAWFRQKPGQSPQLLIYAASTLQSGVSDRFSGSGSGTDFTLRISRLEADDAAVYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 18 - protein, 107 aa, Artificial Sequence
  Caninized variable light chain of D2E7 v2 (KBA VL v2)
DIVMTQTPPSLSASPGEPVSISCRASQGIRNYLAWFRQKPGQSPQLLIYAASTLQSGVSDRFSGSGSGTDFTLRISRLEADDAAVYYCQRYNRAPYTFGQGTKVEIK

SEQ ID NO: 19 - protein, 217 aa, Artificial Sequence
  Caninized variable light chain of D2E7 v2 and canine kappa light constant region
DIVMTQTPPSLSASPGEPVSISCRASQGIRNYLAWFRQKPGQSPQLLIYAASTLQSGVSDRFSGSGSGTDFTLRISRLEADDAAVYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 20 - protein, 15 aa, Artificial Sequence
  Variable light chain framework region LC-FR2 of KBA VL v2
WFRQKPGQSPQLLIY

SEQ ID NO: 21 - protein, 475 aa, Artificial Sequence
  Exemplary caninized variable heavy chain of D2E7 and canine IgG-B constant region with leader
MGWSWIFLFLLSGTAGVHSEVQLVESGGDLVKPGGSLRLSCVASGFTFDDYAMHWVRQAPGKGLQWVSAITWNSGHIDYADAVKGRFTISRDNAKNTLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 22 - protein, 121 aa, Artificial Sequence
  Exemplary caninized variable heavy chain of D2E7 (KBA VH)
EVQLVESGGDLVKPGGSLRLSCVASGFTFDDYAMHWVRQAPGKGLQWVSAITWNSGHIDYADAVKGRFTISRDNAKNTLYLQMNSLRAEDTAVYYCAKVSYLSTASSLDYWGQGTLVTVSS

SEQ ID NO: 23 - protein, 456 aa, Artificial Sequence
  Exemplary caninized variable heavy chain of D2E7 and canine IgG-B constant region
EVQLVESGGDLVKPGGSLRLSCVASGFTFDDYAMHWVRQAPGKGLQWVSAITWNSGHIDYADAVKGRFTISRDNAKNTLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 24 - protein, 239 aa, Artificial Sequence
  Caninized variable light chain of D2E7 v3 and canine kappa light constant region with leader
MDMRVPAQLLGLLLLWLSGARCDIVMTQTPLSLSASPGEPVSISCRASQGIRNYLAWYQQKPGQAPRLLIYAASTLQSGVPDRFSGSGSGTDFTLRIGRLEAEDAAIYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 25 - protein, 107 aa, Artificial Sequence
  Caninized variable light chain of D2E7 v3 (KBA VL v3)
DIVMTQTPLSLSASPGEPVSISCRASQGIRNYLAWYQQKPGQAPRLLIYAASTLQSGVPDRFSGSGSGTDFTLRIGRLEAEDAAIYYCQRYNRAPYTFGQGTKVEIK

SEQ ID NO: 26 - protein, 217 aa, Artificial Sequence
  Caninized variable light chain of D2E7 v3 and canine kappa light constant region
DIVMTQTPLSLSASPGEPVSISCRASQGIRNYLAWYQQKPGQAPRLLIYAASTLQSGVPDRFSGSGSGTDFTLRIGRLEAEDAAIYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 27 - protein, 15 aa, Artificial Sequence
  Variable light chain framework region LC-FR2 of KBA VL v3
WYQQKPGQAPRLLIY

SEQ ID NO: 28 - protein, 239 aa, Artificial Sequence
  Caninized variable light chain of D2E7 v4 and canine kappa light constant region with leader
MDMRVPAQLLGLLLLWLSGARCDIVMTQSPASLSLSQEEKVTITCRASQGIRNYLAWYQQKPGQAPKLLIYAASTLQSGVPSRFSGSGSGTDFSFTISSLEPEDVAVYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 29 - protein, 107 aa, Artificial Sequence
  Caninized variable light chain of D2E7 v4 (KBA VL v4)
DIVMTQSPASLSLSQEEKVTITCRASQGIRNYLAWYQQKPGQAPKLLIYAASTLQSGVPSRFSGSGSGTDFSFTISSLEPEDVAVYYCQRYNRAPYTFGQGTKVEIK

SEQ ID NO: 30 - protein, 217 aa, Artificial Sequence
  Caninized variable light chain of D2E7 v4 and canine kappa light constant region
DIVMTQSPASLSLSQEEKVTITCRASQGIRNYLAWYQQKPGQAPKLLIYAASTLQSGVPSRFSGSGSGTDFSFTISSLEPEDVAVYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 31 - protein, 15 aa, Artificial Sequence
  Variable light chain framework region LC-FR2 of KBA VL v4
WYQQKPGQAPKLLIY

SEQ ID NO: 32 - protein, 239 aa, Artificial Sequence
  Caninized variable light chain of D2E7 v5 and canine kappa light constant region with leader
MDMRVPAQLLGLLLLWLSGARCEIQMTQSPTLLSVSPGDRVTITCQASQGIRNYLAWYQQKRGNAPKCLIYAASTLQSGVPLRFSGSGSGTDFTLTINSLEPKDVATYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 33 - protein, 107 aa, Artificial Sequence
  Caninized variable light chain of D2E7 v5 (KBA VL v5)
EIQMTQSPTLLSVSPGDRVTITCQASQGIRNYLAWYQQKRGNAPKCLIYAASTLQSGVPLRFSGSGSGTDFTLTINSLEPKDVATYYCQRYNRAPYTFGQGTKVEIK

SEQ ID NO: 34 - protein, 217 aa, Artificial Sequence
  Caninized variable light chain of D2E7 v5 and canine kappa light constant region
EIQMTQSPTLLSVSPGDRVTITCQASQGIRNYLAWYQQKRGNAPKCLIYAASTLQSGVPLRFSGSGSGTDFTLTINSLEPKDVATYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 35 - protein, 15 aa, Artificial Sequence
  Variable light chain framework region LC-FR2 of KBA VL v5
WYQQKRGNAPKCLIY

SEQ ID NO: 36 - protein, 239 aa, Artificial Sequence
  Caninized variable light chain of D2E7 v6 and canine kappa light constant region with leader
MDMRVPAQLLGLLLLWLRGARCDIQMTQTPLSLSASPGEPVSISCRASQGIRNYLAWYLQKPGQSPRLLIYAASTLQSGVPDRFSGSGSGTDFTLRIGRLEAEDAAIYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 37 - protein, 107 aa, Artificial Sequence
  Caninized variable light chain of D2E7 v6 (KBA VL v6)
DIQMTQTPLSLSASPGEPVSISCRASQGIRNYLAWYLQKPGQSPRLLIYAASTLQSGVPDRFSGSGSGTDFTLRIGRLEAEDAAIYYCQRYNRAPYTFGQGTKVEIK

SEQ ID NO: 38 - protein, 217 aa, Artificial Sequence
  Caninized variable light chain of D2E7 v6 and canine kappa light constant region
DIQMTQTPLSLSASPGEPVSISCRASQGIRNYLAWYLQKPGQSPRLLIYAASTLQSGVPDRFSGSGSGTDFTLRIGRLEAEDAAIYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 39 - protein, 15 aa, Artificial Sequence
  Variable light chain framework region LC-FR2 of KBA VL v6
WYLQKPGQSPRLLIY

SEQ ID NO: 40 - protein, 234 aa, Artificial Sequence [sequence omitted]

SEQ ID NO: 41 - protein, 214 aa, Artificial Sequence
  Chimeric variable light chain of D2E7 and canine lambda light constant region
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 237 aa, Artificial Sequence
  Caninized variable light chain of D2E7 v7 and canine kappa light constant region with leader
METDTLLLWVLLLWVPGSTGDIVMTQSPASLSASPEEKVTITCRASQGIRNYLAWYQQKPGKAPKLLIYAASTLQSGVPSRFSGSGSGTDFTLTISSLEPEDVAVYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 43 - protein, 107 aa, Artificial Sequence
  Caninized variable light chain of D2E7 v7 (KBA VL v7)
DIVMTQSPASLSASPEEKVTITCRASQGIRNYLAWYQQKPGKAPKLLIYAASTLQSGVPSRFSGSGSGTDFTLTISSLEPEDVAVYYCQRYNRAPYTFGQGTKVEIK

SEQ ID NO: 44 - protein, 217 aa, Artificial Sequence
  Caninized variable light chain of D2E7 v7 and canine kappa light constant region
DIVMTQSPASLSASPEEKVTITCRASQGIRNYLAWYQQKPGKAPKLLIYAASTLQSGVPSRFSGSGSGTDFTLTISSLEPEDVAVYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 45 - protein, 15 aa, Artificial Sequence
  Variable light chain framework region LC-FR2 of KBA VL v7
WYQQKPGKAPKLLIY

SEQ ID NO: 46 - protein, 107 aa, Artificial Sequence
  Caninized variable light chain of D2E7 v8
DIVMTQTPLSLSVSPGEPASISCRASQGIRNYLAWYQQKPGKAPKLLIYAASTLQSGVPDRFSGSGSGTDFTLRIGRVEAEDAGIYYCQRYNRAPYTFGQGTRLEVR

SEQ ID NO: 47 - protein, 107 aa, Artificial Sequence
  Variable light chain of mouse clone cA2 (Infliximab)
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 48 - protein, 120 aa, Artificial Sequence
  Variable heavy chain of mouse clone cA2 (Infliximab)
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSS

SEQ ID NO: 49 - protein, 11 aa, Artificial Sequence
  Variable light chain CDR L1 of cA2
RASQFVGSSIH

SEQ ID NO: 50 - protein, 7 aa, Artificial Sequence
  Variable light chain CDR L2 of cA2
YASESMS

SEQ ID NO: 51 - protein, 9 aa, Artificial Sequence
  Variable light chain CDR L3 of cA2
QQSHSWPFT

SEQ ID NO: 52 - protein, 5 aa, Artificial Sequence
  Variable heavy chain CDR H1 of cA2
NHWMN

SEQ ID NO: 53 - protein, 19 aa, Artificial Sequence
  Variable heavy chain CDR H2 of cA2
EIRSKSINSATHYAESVKG

SEQ ID NO: 54 - protein, 9 aa, Artificial Sequence
  Variable heavy chain CDR H3 of cA2
NYYGSTYDY

SEQ ID NO: 55 - protein, 107 aa, Artificial Sequence
  Caninized variable light chain of cA2 v1
DIVMTQTPLSLSVSPGETASISCRASQFVGSSIHWFRQKPGQSPQRLIKYASESMSGVPDRFSGSGSGTDFTLRINTVEADDTGVYYCQQSHSWPFTFGSGTKLEIK

SEQ ID NO: 56 - protein, 107 aa, Artificial Sequence
  Caninized variable light chain of cA2 v2
DIVMTQTPLSLSVSPGETVSFSCRASQFVGSSIHWFRQKPGQSPQLLIKYASESMSGIPDRFSGSGSGTDFTLSINTVEADDIAVYYCQQSHSWPFTFGSGTKLEIK

SEQ ID NO: 57 - protein, 120 aa, Artificial Sequence
  Caninized variable heavy chain of mouse clone cA2 v1
EVQLVESGGDLVKPGGSLRLSCVASGFIFSNHWMNWVRQAPGKGLQWVAEIRSKSINSATHYAESVKGRFTISRDNARNTLYLQMNSLRSEDTGVYYCSRNYYGSTYDYWGQGTTVTVSS

SEQ ID NO: 58 - protein, 120 aa, Artificial Sequence
  Caninized variable heavy chain of mouse clone cA2 v2
EVQLVESGGGLVKPGGSLRLSCVASGFIFSNHWMNWVRQSPGKGLQWVAEIRSKSINSATHYAESVKGRFTISRDNARNTVYLQMNSLRSEDTGVYYCSRNYYGSTYDYWGQGTTVTVSS

SEQ ID NO: 59 - protein, 217 aa, Artificial Sequence
  Caninized variable light chain of cA2 v1 and canine kappa light constant region
DIVMTQTPLSLSVSPGETASISCRASQFVGSSIHWFRQKPGQSPQRLIKYASESMSGVPDRFSGSGSGTDFTLRINTVEADDTGVYYCQQSHSWPFTFGSGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 60 - protein, 217 aa, Artificial Sequence
  Caninized variable light chain of cA2 v2 and canine kappa light constant region
DIVMTQTPLSLSVSPGETVSFSCRASQFVGSSIHWFRQKPGQSPQLLIKYASESMSGIPDRFSGSGSGTDFTLSINTVEADDIAVYYCQQSHSWPFTFGSGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 61 - protein, 455 aa, Artificial Sequence
  Caninized variable heavy chain of cA2 v1 and canine IgG-B constant region
EVQLVESGGDLVKPGGSLRLSCVASGFIFSNHWMNWVRQAPGKGLQWVAEIRSKSINSATHYAESVKGRFTISRDNARNTLYLQMNSLRSEDTGVYYCSRNYYGSTYDYWGQGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 62 - protein, 455 aa, Artificial Sequence
  Caninized variable heavy chain of cA2 v2 and canine IgG-B constant region
EVQLVESGGGLVKPGGSLRLSCVASGFIFSNHWMNWVRQSPGKGLQWVAEIRSKSINSATHYAESVKGRFTISRDNARNTVYLQMNSLRSEDTGVYYCSRNYYGSTYDYWGQGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 63 - protein, 475 aa, Artificial Sequence
  Exemplary caninized variable heavy chain of D2E7 and variant canine IgG-B (FcRn long-acting Q to Y) constant region with leader
MGWSWIFLFLLSGTAGVHSEVQLVESGGDLVKPGGSLRLSCVASGFTFDDYAMHWVRQAPGKGLQWVSAITWNSGHIDYADAVKGRFTISRDNAKNTLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 64 - protein, 456 aa, Artificial Sequence
  Exemplary caninized variable heavy chain of D2E7 and variant canine IgG-B (FcRn long-acting Q to Y) constant region
EVQLVESGGDLVKPGGSLRLSCVASGFTFDDYAMHWVRQAPGKGLQWVSAITWNSGHIDYADAVKGRFTISRDNAKNTLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 65 - protein, 11 aa, Artificial Sequence
  Variable light chain CDR L1 of alphaD11
RASEDIYNALA

SEQ ID NO: 66 - protein, 8 aa, Artificial Sequence
  Variable light chain CDR L2 of alphaD11
YNTDTLHT

SEQ ID NO: 67 - protein, 9 aa, Artificial Sequence
  Variable light chain CDR L3 of alphaD11
QHYFGYPRT

SEQ ID NO: 68 - protein, 13 aa, Artificial Sequence
  Variable heavy chain CDR H1 of alphaD11
TVSGFSLTNNNVN

SEQ ID NO: 69 - protein, 9 aa, Artificial Sequence
  Variable heavy chain CDR H2 of alphaD11
GVWAGGATD

SEQ ID NO: 70 - protein, 16 aa, Artificial Sequence
  Variable heavy chain CDR H3 of alphaD11
ARDGGYSSSTLYAMDA

SEQ ID NO: 71 - protein, 107 aa, Artificial Sequence
  Variable light chain of rat alphaD11
DIQMTQSPASLSASLGETVTIECRASEDIYNALAWYQQKPGKSPQLLIYNTDTLHTGVPSRFSGSGSGTQYSLKINSLQSEDVASYFCQHYFGYPRTFGGGTKLELK

SEQ ID NO: 72 - protein, 122 aa, Artificial Sequence
  Variable heavy chain of rat alphaD11
QVQLKESGPGLVQPSQTLSLTCTVSGFSLTNNNVNWVRQATGRGLEWMGGVWAGGATDYNSALKSRLTITRDTSKSQVFLKMHSLQSEDTATYYCARDGGYSSSTLYAMDAWGQGTTVTVSS

SEQ ID NO: 73 - protein, 107 aa, Artificial Sequence
  Exemplary caninized variable light chain of alphaD11
EIVMTQSPGSLAGSAGESVSINCRASEDIYNALAWYQQKPGERPKLLIYNTDTLHTGVPARFSSSGSGTDFTLTINNLQAEDVGDYFCQHYFGYPRTFGAGTKVELK

SEQ ID NO: 74 - protein, 122 aa, Artificial Sequence
  Exemplary caninized variable heavy chain of alphaD11
EVQLVESGGDLVKPAGSLRLSCVASGFSLTNNNVNWVRQAPGKGLEWMGGVWAGGATDYNSALKSRFTISRDNAKNTLYLQMNSLRAEDTAVYYCARDGGYSSSTLYAMDAWGQGTLVTVSS

SEQ ID NO: 75 - protein, 13 aa, Artificial Sequence
  Variable heavy chain CDR H1 of caninized alphaD11
VASGFSLTNNNVN

SEQ ID NO: 76 - protein, 237 aa, Artificial Sequence
  Exemplary caninized variable light chain of alphaD11and canine kappa light constant region with leader
METDTLLLWVLLLWVPGSTGEIVMTQSPGSLAGSAGESVSINCRASEDIYNALAWYQQKPGERPKLLIYNTDTLHTGVPARFSSSGSGTDFTLTINNLQAEDVGDYFCQHYFGYPRTFGAGTKVELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 77 - protein, 217 aa, Artificial Sequence
  Exemplary caninized variable light chain of alphaD11and canine kappa light constant region
EIVMTQSPGSLAGSAGESVSINCRASEDIYNALAWYQQKPGERPKLLIYNTDTLHTGVPARFSSSGSGTDFTLTINNLQAEDVGDYFCQHYFGYPRTFGAGTKVELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 78 - protein, 476 aa, Artificial Sequence
  Exemplary caninized variable heavy chain of alphaD11and variant canine IgG-B (FcRn long-acting Q to Y, C1q-, CD16-) constant region with leader
MAVLGLLLCLVTFPSCVLSEVQLVESGGDLVKPAGSLRLSCVASGFSLTNNNVNWVRQAPGKGLEWMGGVWAGGATDYNSALKSRFTISRDNAKNTLYLQMNSLRAEDTAVYYCARDGGYSSSTLYAMDAWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHYDWLKGKQFTCRVNNIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 79 - protein, 457 aa, Artificial Sequence
  Exemplary caninized variable heavy chain of alphaD11and variant canine IgG-B (FcRn long-acting Q to Y, C1q-, CD16-) constant region
EVQLVESGGDLVKPAGSLRLSCVASGFSLTNNNVNWVRQAPGKGLEWMGGVWAGGATDYNSALKSRFTISRDNAKNTLYLQMNSLRAEDTAVYYCARDGGYSSSTLYAMDAWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHYDWLKGKQFTCRVNNIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 80 - protein, 729 aa, Artificial Sequence
  Exemplary bi-specific NGF and TNF antibody with leader (Anti-NGF + Fc IgGB (FcRn long-acting Q to Y, C1q-, CD16-) + anti-TNF ScFv)
MAVLGLLLCLVTFPSCVLSEVQLVESGGDLVKPAGSLRLSCVASGFSLTNNNVNWVRQAPGKGLEWMGGVWAGGATDYNSALKSRFTISRDNAKNTLYLQMNSLRAEDTAVYYCARDGGYSSSTLYAMDAWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHYDWLKGKQFTCRVNNIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGKGGGGSGGGEVQLVESGGDLVKPGGSLRLSCVASGFTFDDYAMHWVRQAPGKGLQWVSAITWNSGHIDYADAVKGRFTISRDNAKNTLYLQMNSLRAEDTAVYYCAKVSYLSTASSLDYWGQGTLVTVSSGGGGGSGGGGSGGGGGSDIVMTQSPASLSASPEEKVTITCRASQGIRNYLAWYQQKPGKAPKLLIYAASTLQSGVPSRFSGSGSGTDFTLTISSLEPEDVAVYYCQRYNRAPYTFGQGTKVEIK

SEQ ID NO: 81 - protein, 710 aa, Artificial Sequence
  Exemplary bi-specific NGF and TNF antibody (Anti-NGF + Fc IgGB (FcRn long-acting Q to Y, C1q-, CD16-) + anti-TNF ScFv)
EVQLVESGGDLVKPAGSLRLSCVASGFSLTNNNVNWVRQAPGKGLEWMGGVWAGGATDYNSALKSRFTISRDNAKNTLYLQMNSLRAEDTAVYYCARDGGYSSSTLYAMDAWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHYDWLKGKQFTCRVNNIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGKGGGGSGGGEVQLVESGGDLVKPGGSLRLSCVASGFTFDDYAMHWVRQAPGKGLQWVSAITWNSGHIDYADAVKGRFTISRDNAKNTLYLQMNSLRAEDTAVYYCAKVSYLSTASSLDYWGQGTLVTVSSGGGGGSGGGGSGGGGGSDIVMTQSPASLSASPEEKVTITCRASQGIRNYLAWYQQKPGKAPKLLIYAASTLQSGVPSRFSGSGSGTDFTLTISSLEPEDVAVYYCQRYNRAPYTFGQGTKVEIK

SEQ ID NO: 82 - protein, 729 aa, Artificial Sequence
  Exemplary bi-specific NGF and TNF antibody with leader (Anti-TNF + Fc IgGB (FcRn long-acting Q to Y, C1q-, CD16-) + anti-NGF ScFv)
MAVLGLLLCLVTFPSCVLSEVQLVESGGDLVKPGGSLRLSCVASGFTFDDYAMHWVRQAPGKGLQWVSAITWNSGHIDYADAVKGRFTISRDNAKNTLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHYDWLKGKQFTCRVNNIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGKGGGGSGGGEVQLVESGGDLVKPAGSLRLSCVASGFSLTNNNVNWVRQAPGKGLEWMGGVWAGGATDYNSALKSRFTISRDNAKNTLYLQMNSLRAEDTAVYYCARDGGYSSSTLYAMDAWGQGTLVTVSSGGGGGSGGGGSGGGGGSEIVMTQSPGSLAGSAGESVSINCRASEDIYNALAWYQQKPGERPKLLIYNTDTLHTGVPARFSSSGSGTDFTLTINNLQAEDVGDYFCQHYFGYPRTFGAGTKVELK

SEQ ID NO: 83 - protein, 710 aa, Artificial Sequence
  Exemplary bi-specific NGF and TNF antibody (Anti-TNF + Fc IgGB (FcRn long-acting Q to Y, C1q-, CD16-) + anti-NGF ScFv)
EVQLVESGGDLVKPGGSLRLSCVASGFTFDDYAMHWVRQAPGKGLQWVSAITWNSGHIDYADAVKGRFTISRDNAKNTLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHYDWLKGKQFTCRVNNIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGKGGGGSGGGEVQLVESGGDLVKPAGSLRLSCVASGFSLTNNNVNWVRQAPGKGLEWMGGVWAGGATDYNSALKSRFTISRDNAKNTLYLQMNSLRAEDTAVYYCARDGGYSSSTLYAMDAWGQGTLVTVSSGGGGGSGGGGSGGGGGSEIVMTQSPGSLAGSAGESVSINCRASEDIYNALAWYQQKPGERPKLLIYNTDTLHTGVPARFSSSGSGTDFTLTINNLQAEDVGDYFCQHYFGYPRTFGAGTKVELK